(19) United States
(12) Patent Application Publication
(10) Pub. No.: US 2025/0009918 A1
(43) Pub. Date: Jan. 9, 2025

(54) NANOPARTICLES FOR POTENTIATING EFFECTS OF RADIATION THERAPY ON ANTI-CANCER IMMUNOTHERAPY

(71) Applicant: Wisconsin Alumni Research Foundation, Madison, WI (US)

(72) Inventors: Shaoqin Gong, Middleton, WI (US); Ying Zhang, Madison, WI (US); Zachary Morris, Madison, WI (US); Raghava N. Sriramaneni, Madison, WI (US)

(73) Assignee: Wisconsin Alumni Research Foundation, Madison, WI (US)

(21) Appl. No.: 18/712,655

(22) PCT Filed: Nov. 22, 2022
(86) PCT No.: PCT/US2022/050793

§ 371 Date: May 22, 2024

Related U.S. Application Data
(60) Provisional application No. 63/282,987, filed on Nov. 24, 2021.

(51) Int. Cl. A61K 51/12; A61K 38/20; A61K 45/06; A61K 47/54; A61K 47/64; A61K 47/69; A61K 49/00; A61K 51/04; A61N 5/10; A61P 35/00

(52) U.S. Cl. CPC A61K 51/1244; A61K 38/2013; A61K 45/06; A61K 47/549; A61K 47/6455; A61K 47/6923; A61K 49/0002; A61K 51/0497; A61N 5/1077; A61P 35/00; A61K 2123/00; A61N 2005/1098

(57) ABSTRACT

The present technology provides nanoparticles comprising a positively charged polymer (e.g., polylysine) electrostatically bound to iron oxide nanoparticles and CpG oligodeoxy nucleotide. Further provided are compositions comprising same and methods of sensitizing tumor cells to radiation therapy, methods of stimulating antigen presenting cells, methods of enhancing stimulation of a type I interferon, and methods of treatment using said nanoparticles and compositions.

### CROSS-REFERENCE TO RELATED PATENT APPLICATIONS

This application claims the benefit of and priority of U.S. Provisional Patent Application 63/282,987, filed Nov. 24, 2021, the entire contents of which is incorporated herein by reference in its entirety.

### FIELD

The present technology relates generally to compositions and methods for in situ tumor vaccination using nanoparticles. Methods of preparing the nanoparticles are also provided.

### BACKGROUND

Cancer immunotherapy has revolutionized clinical oncology, and immune checkpoint blockade (ICB) therapies have become one of the most effective cancer treatments. However, many cancer patients do not respond to this treatment, particularly those with immunologically “cold” tumors that are commonly characterized by low neo-antigen load and limited immune cell infiltration. In situ cancer vaccination aims to convert a patient's own tumor into a locus for presentation of tumor-specific antigens in a way that will stimulate and diversify an anti-tumor T cell response. Such approaches can improve the response rates for ICB therapies by promoting antigen presentation and tumor-specific T cell response—an effect that can enhance anti-tumor response in tumors that are immunologically “hot” and in those that are immunologically “cold”.

In certain circumstances, radiation therapy (RT) has proven capable of activating an in situ vaccine response to tumors. By stimulating immunogenic cell death, increasing tumor infiltration by immune cells, and enhancing the susceptibility of tumor cells to immune-mediated killing, RT has been shown in some preclinical models to augment response to ICBs. Clinical studies indicate that such effects may be achieved in cancer patients but also suggest that more potent therapy will be needed in order to activate anti-tumor immune responses in combination with ICBs for the majority of patients with metastatic cancers. This may reflect the fact that although radiation may elicit many effects in the tumor microenvironment that are conducive to augmenting the development and propagation of adaptive anti-tumor immunity, it can also activate detrimental effects such as the recruitment and activation of suppressive immune cell lineages. For example, anti-inflammatory tumor associated macrophages (TAMs) such as M2 macrophages predominate (e.g., over pro-inflammatory TAMs such as M1 macrophages that have tumoricidal effects) in many tumors, leading to an immunosuppressive microenvironment that promote immune evasion, contributing to the “cold” phenotype and diminishing the efficacy of cancer immunotherapies.

### SUMMARY OF THE INVENTION

As disclosed herein, the present technology provides nanoparticles (PIC NP) that potentiate the “in situ vaccination” effect of RT and improve response to ICB therapy. In various aspects and embodiments, the PIC NPs exhibit one or more of the following activities: 1) sensitize tumor cells to RT; 2) facilitate the uptake of tumor associated antigens (TAA) by antigen presenting cells (APCs); 3) stimulate the maturation of APCs to activate CD8+ and CD4+ T cell responses; 4) enhance RT-mediated activation of a type I interferon response downstream of cGAS/STING; 5) diminish M2 macrophage tumor infiltration; and 6) increase tumor infiltration by M1 macrophages. As such, the PIC NP thereby enhances the development of an immune response against tumors—including those that are immunologically “cold”.

Thus, in one aspect, the present technology provides a nanoparticle that includes a positively charged polymer having a plurality of positive charges (e.g., polylysine (PLL)) electrostatically bound to iron oxide nanoparticles (ION) and CpG oligodeoxynucleotide. In any embodiments, the positively charged polymer (e.g., PLL) may have a molecular weight of about 5 kDa to about 150 kDa. In any embodiments, the ION is Fe₃O₄. In any embodiments, the nanoparticle has a hydrodynamic diameter from about 60 to about 200 nm.

In another aspect, the present technology provides methods of sensitizing tumor cells to radiation therapy and/or creating radiation dose heterogeneity in a radiated tumor microenvironment comprising administering an effective amount of any nanoparticle disclosed herein.

In still another aspect, the present technology provides methods of stimulating antigen presenting cells comprising administering an effective amount of any nanoparticle disclosed herein to the tumor cells.

In still another aspect, the present technology provides methods of enhancing stimulation of a type I interferon response comprising administering an effective amount of any nanoparticle disclosed herein to the tumor cells.

In still another aspect, the present technology provides methods of increasing the ratio of M1:M2 macrophages infiltration in radiated tumors comprising administering an effective amount of any nanoparticle disclosed herein to the tumor cells.

In still another aspect, the present technology provides methods of treatment comprising administering to a subject suffering from cancer (e.g., an immunologically hot or an immunologically cold cancer) an effective amount of any nanoparticle disclosed herein and an effective amount of radiation therapy. In any embodiments, the methods of treatment further include administering an effective amount of a checkpoint inhibitor and/or immune adjuvant to the subject. In any embodiments, the cancer may be selected from the group consisting of breast cancer, bladder cancer, cervical cancer, colon cancer, head and neck cancer, liver cancer, lung cancer, pancreatic cancer, prostate cancer, renal cell cancer, melanoma, sarcomas, stomach cancer, rectal cancer, and Hodgkin lymphoma.

The foregoing summary is illustrative only and is not intended to be in any way limiting. In addition to the illustrative aspects, embodiments and features described above, further aspects, embodiments and features will become apparent by reference to the following drawings and the detailed description.

### BRIEF DESCRIPTION OF THE DRAWINGS

FIGS. 1A-1L (1A) An illustrative embodiment of the preparation procedure of PIC NP. (1B) Proposed mechanisms of action for PIC NP in potentiating the in situ vaccination effect of RT. PIC NP is designed to sensitize tumor cells to RT, capture tumor-associated antigens (TAAs) released from dying tumor cells, promote the uptake of these captured TAAs in antigen presenting cells (APCs), activate and mature APCs, promote a favorable ratio of M1:M2 TAMs, and increase tumor infiltration by APCs and effector T cells and increase tumor infiltration by APCs and effector T cells in part by more effectively activating a type I interferon response among radiated cells. (1C) As a result of these mechanisms, PIC NP combined with RT can help convert a radiated immunologically “cold” tumor to an immunologically “hot” microenvironment. (1D) Hydrodynamic size distribution of PIC. (1E) Transmission electron microscopy image of PIC. Scale bar: 100 nm. (1F) Zeta potentials of ION and PIC. (n=3 independent samples). (1G) CpG complexation abilities in different formulations, as measured by agarose gel electrophoresis. (1H) The particle size stability of PIC during storage at 4° C. (n=3 independent samples). (1I) The protein concentrations in B78 cell lysates after incubation with PIC (0.14 mg/mL) for 4 hours. (n=3 biologically independent samples). (1J) Clonogenic assay of B78 melanoma cells after treatment with PIC (4.67 μg/mL) and indicated radiation doses. (n=3 biologically independent samples). PIC was added to the cells 4 hours before radiation, and fresh culture media was exchanged for this PIC treatment media 1 hour after radiation. The colonies were counted at day 7. (1K) The immunofluorescence images of B78 cells after indicated treatment (RT: 12 Gy; PIC: 4.67 μg/mL). (1L) Quantification of foci of γH2AX as shown in (1K). 50 cells in each group were analyzed with ImageJ. PIC was added to the cells 4 hours before radiation, and fresh culture media was exchanged for PIC treatment media 1 hour after radiation. Statistical significance was calculated via un-paired t-test in 1I, and one-way ANOVA test in 1J and 1L. Data in 1F, 1H, 1I, 1J and 1L are presented as mean±SD. *p<0.05, **p<0.01, ***p<0.001 and ****p<0.0001. A representative image of three independent samples from each group is shown in 1C, 1E and 1I.

FIGS. 2A-2J illustrate the immunomodulatory effect of PIC in vitro. (2A) Scheme for the RT-qPCR studies of B78 cells. The relative mRNA expression of (2B) Ifnβ1 and (2C) Pd-l1 in B78 cells at day 7 after initiation of indicated treatments. PIC: 4.67 μg/mL; RT: 12 Gy. (n=3 biologically independent samples). (2D) Scheme for the study of the macrophage polarization effect of PIC on BMDMs. The MFI of CD206 and CD163 on CD11b⁺F4/80⁺ BMDMs at day 1 and day 4 after the cells were treated with (2E) PIC and (2F) RT+PIC. (2G) The ratios of CD80 MFI to CD206 MFI on CD11b⁺F4/80⁺ BMDMs at day 1 and day 4 after indicated treatment. (2H) The ratios of M1:M2 macrophage in CD11b⁺F4/80⁺ BMDMs at day 4 after indicated treatment. M1-like: CD80⁺CD206⁻; M2-like: CD206⁺CD80⁻. PIC: 4.67 μg/mL; RT: 12 Gy. (n=4 biologically independent samples). (2I) The MFI of CD80 and CD86 on CD317⁺CD11c⁺ pDCs after indicated treatments. PIC: 4.67 μg/mL; CpG: 0.5 μg/mL. (n=3 biologically independent samples). (2J) The percentage of FITC-Ova positive cells among CD11c⁺ DCs at 24 h after treatment. FITC-Ova: 1.67 μg/mL; PIC: 4.67 μg/mL. (n=4 biologically independent samples). DCs were enriched from the splenocytes collected from Flt3L treated C57BL/6 mice. Statistical significance was calculated via one-way ANOVA test in 2B-2C and 2E-2J, and data are presented as mean±SD. *p<0.05, **p<0.01, ***p<0.001 and ****p<0.0001.

FIGS. 3A-3E illustrate the biodistribution and immunomodulatory effects of PIC in vivo. The cellular uptake of Cy5-PIC in different cells within the (3A) tumors and (3B) TDLNs after the Cy5-PIC was intratumorally injected into B78 melanoma bearing mice. (n=5 biologically independent samples). The gating strategy is provided in FIG. 21. (3C) Scheme for the study of the immunomodulation effect of PIC+RT on the B78 tumor microenvironment. (3D) The mRNA expression of select inflammatory and anti-inflammatory genes (Ifnβ1, Mx1, Nos2, Arg1, Ifnγ, Il6, Tnfα, Il1β, Il10 and Tgfβ1) in B78 tumors at day 15 after initiation of indicated treatments. (3E) The mRNA expression ratios of Nos2:Arg1, Tnfα: Tgfβ1 and Il1β: Il10 in tumors at day 15 after indicated treatments. PIC: 140 μg/100 μL/dose; RT: 12 Gy. (n=5 biologically independent samples). Data in 3A-3B and 3D-3E are shown as mean±SD. Statistical significance was calculated via un-paired t-test in 3A-3B, and one-way ANOVA test in 3D-3E. *p<0.05, **p<0.01, ***p<0.001 and ****p<0.0001.

FIGS. 4A-4P illustrate the influence of PIC on tumor-infiltrating immune cell populations. (4A) Scheme for the studies. The percentage of (4B) CD80⁺CD206⁻ M1-like macrophage and (4C) CD206⁺CD80⁻ M2-like macrophage in CD11b⁺F4/80⁺ macrophages in B78 tumors. (4D) The ratios of M1:M2 macrophages in B78 tumors after indicated treatments. The percentage of (4E) CD103⁺CD11b⁻ cDC1s and (4F) CD11b⁺CD103⁻ cDC2s in CD11c⁺MHCII⁺ DCs in B78 tumors. (4G) The percentage of CD317⁺ pDCs in CD11c⁺ cells in B78 tumors. (4H) The percentage of CD80⁺ cells in CD317⁺ CD11c⁺ pDCs in B78 tumors. The percentage of (4I) CD8⁺ and (4J) CD4⁺ cells in CD45⁺CD3⁺ T cells in B78 tumors. (4K) The percentage of CD25⁺FOXP3⁺ Tregs in CD4⁺ T cells in B78 tumors. (4L) The percentage of CD69⁺CD4⁺ and CD44⁺CD4⁺ cells in CD45⁺CD3⁺ T cells in B78 tumors. (4M) The percentage of CD69⁺CD8⁺ and CD44⁺CD8⁺ cells in CD45⁺CD3⁺ T cells in B78 tumors. The MFI of PD-1 on (4N) CD3⁺CD4⁺ T cells and (4O) CD3⁺CD8⁺ T cells in B78 tumors. (n=5 biologically independent samples for 4B-4O). (4P) The percentage of central memory T cells (CD44⁺CD62L⁺), effector memory T cells (CD44⁺CD62L⁻), naive T cells (CD44⁻CD62L⁺) and others (CD44⁻CD62L⁻) among CD3⁺CD4⁺ T cells (left) and CD3⁺CD8⁺ T cells (right) in TDLNs. PIC: 140 μg/100 μL/dose; RT: 12 Gy. The gating strategies are provided in FIGS. 26-27. Statistical significance was calculated via one-way ANOVA test in 4B-4O, and data are shown as mean±SD. *p<0.05, **p<0.01, ***p<0.001 and ****p<0.0001.

FIGS. 5A-5I illustrate the triple combination of PIC+RT+anti-CTLA-4 (C4) improves tumor response and mouse survival leading to anti-tumor memory in mice bearing a B78 melanoma. (5A) Scheme for the studies. (5B) Average tumor growth curves and (5C) survival rate of mice after the treatments indicated. (Control: n=10; RT: n=11; C4 and PIC+C4: n=7; PIC+RT+C4: n=13; others: n=12 biologically independent animals). (5D) Individual tumor growth curves in (5B). (5E) Average tumor growth curves after the naïve and tumor-free mice were re-challenged with B78 melanoma cells. (n=5 biologically independent animals). (5F) Individual tumor growth curves in (5E). (5G) Scheme for the co-culture of splenocytes extracted from tumor re-challenged mice with B16 melanoma cells. Quantification of (5H) CD69⁺ and (5I) GZMB⁺ in CD4⁺ and CD8⁺ T cells in splenocytes by flow cytometry. (n=6 biologically independent samples). Control: non-radiated B16 cells co-cultured with splenocytes from naïve mice; Re-challenge: non-radiated B16 cells co-cultured with splenocytes from re-challenged mice; Re-challenge+RT: radiated B16 cells co-cultured with splenocytes from re-challenged mice. CR: complete response. C4: anti-CTLA-4. i.p.: intraperitoneal injection. PIC: 140 μg/100 μL/dose. C4: 100 μg/100 μL/dose. Data in 5B, 5E, 5H and 5I are shown as mean±SD. Statistical significance was calculated via linear mixed effects modeling in 5B, log rank test in 5C, time-weighted average in 5E, and one-way ANOVA test in 5H-5I. *p<0.05, **p<0.01, ***p<0.001 and ****p<0.0001.

FIGS. 6A-6G illustrate the combination of PIC+RT+anti-CTLA-4 (C4) improves tumor response and survival in both the MyC-CaP prostate and TC11 breast tumor models. (6A) Scheme for the studies. (6B) Average tumor growth curves of MyC-CaP prostate tumors and (6C) survival rate of mice after the indicated treatments. (n=8 biologically independent animals). (6D) Individual tumor growth curves in (6B). (6E) Average tumor growth curves of TC11 tumor and (6F) survival rate of mice after the indicated treatments (control: n=11; PIC+RT+C4: n=12; others: n=10 biologically independent animals). (6G) Individual tumor growth curves in (6E). CR: complete response. C4: anti-CTLA-4. i.p.: intraperitoneal injection. For MyC-CaP prostate cancer model, tumor was implanted on the right flank of mice. For TC11 breast cancer model, tumor was implanted on the right mammary fat pad of mice. PIC: 140 μg/100 μL/dose. C4: 100 μg/100 μL/dose. Data in 6B and 6E are shown as mean±SD. Statistical significance was calculated via linear mixed effects modeling in 6B and 6E, and log rank test in 6C and 6F. *p<0.05, **p<0.01, ***p<0.001 and ****p<0.0001.

FIGS. 7A-7D illustrate the systemic anti-tumor immune response induced by PIC+RT+anti-CTLA-4 (C4). (7A) Scheme for the study of the abscopal effect of PIC+RT+anti-CTLA-4. (7B) Average tumor growth curves of both right and left tumor, and (7C) survival rate of mice after the indicated treatment regimen. (n=9 biologically independent animals). (7D) Individual tumor growth curves in (7B). CR: complete response. C4: anti-CTLA-4. i.p.: intraperitoneal injection. PIC: 140 μg/100 μL/dose. C4: 100 μg/100 μL/dose. Data in 7B are shown as mean±SD. The statistical significance was calculated via linear mixed effects modeling in 7B, and log-rank test in 7C. *p<0.05, **p<0.01, ***p<0.001 and ****p<0.0001. Only the PIC+RT+C4 group was compared with others to specifically test the hypothesis that this combination treatment would achieve greater tumor response and survival compared to doublet combination or control groups.

FIG. 8 [Reserved]

FIG. 9 shows a TEM image of an illustrative embodiment of an ION. Scale bar: 50 nm. A representative image from three independent samples is shown.

FIGS. 10A-10C (10A) The particle size of PIC in the presence of 1 mM PBS during storage at 4° C. The (10B) particle size and (10C) zeta potential of lyophilized PIC in the presence of 1% sucrose during storage for 12 weeks at −20° C. (n=3 independent samples). L: PIC after lyophilization. Data are shown as mean±SD. Statistical significance was calculated via one-way ANOVA test in 10B-10C. *p<0.05, **p<0.01, ***p<0.001 and ****p<0.0001.

FIGS. 11A-11B The cell viability of (11A) B78 cells and (11B) RAW264.7 cells after in vitro co-culture with indicated concentrations of PIC for 48 h. (n=4 biologically independent samples). Data are shown as mean±SD. Statistical significance was calculated via one-way ANOVA test. *p<0.05, **p<0.01, ***p<0.001 and ****p<0.0001.

FIGS. 12A-12B The mean fluorescence intensity (MFI) of FITC in (12A) B78 cells and (12B) RAW264.7 cells after in vitro treatment with indicated concentrations of FITC-labeled PIC for 2 h. (n=4 biologically independent samples). Data are shown as mean±SD. Statistical significance was calculated via one-way ANOVA test. *p<0.05, **p<0.01, ***p<0.001 and ****p<0.0001.

FIG. 13 Confocal laser scanning microscopy (CLSM) images of B78 cells and RAW264.7 cells after treatment with FITC-labeled PIC for 2 h. A representative image of three independent samples from each group is shown.

FIGS. 14A-14B The mRNA expression of (14A) Ifnβ1 and (14B) Pd-l1 in B78 cells at day 1 and day 4 after indicated in vitro treatments. (n=3 biologically independent samples). PIC: 4.67 μg/mL; RT: 12 Gy. The treatments of the cells were given per FIG. 2A. Data are shown as mean±SD. Statistical significance was calculated via one-way ANOVA test. *p<0.05, **p<0.01, ***p<0.001 and ****p<0.0001.

FIGS. 15A-15B The (15A) mean fluorescence intensity (MFI) of CD80 and (15B) ratios of CD80 MFI to CD163 MFI on CD11b⁺F4/80⁺ BMDMs at day 1 and day 4 after indicated treatments. RT: 12 Gy. PIC: 4.67 μg/mL. The treatments of the cells were given per FIG. 2D. (n=4 biologically independent samples). Data are shown as mean±SD. Statistical significance was calculated via one-way ANOVA test. *p<0.05, **p<0.01, ***p<0.001 and ****p<0.0001.

FIGS. 16A-16D (16A) Representative flow cytometry data and (16B) quantification of M1-like macrophages (CD80⁺CD206⁻) and M2-like macrophages (CD206⁺CD80⁻) among CD11b⁺F4/80⁺ BMDMs and their ratios at day 1 after indicated treatments. (16C) Representative flow cytometry data and (16D) quantification of the M1-like macrophages (CD80⁺CD206⁻) and M2 macrophages (CD206⁺CD80⁻) among CD11b⁺F4/80⁺ BMDMs at day 4 after indicated treatments. (n=4 biologically independent samples). PIC: 4.67 μg/mL; RT: 12 Gy. The treatments of the cells were given per FIG. 2D. Statistical significance was calculated via one-way ANOVA test in 16B and 16D, and data are shown as mean±SD. *p<0.05, **p<0.01, ***p<0.001 and ****p<0.0001.

FIGS. 17A-17B (17A) The concentration of IFN-β secreted from CD11c⁺ dendritic cells after indicated treatment for 24 hours. CpG: 0.5 μg/mL; PIC: 4.67 μg/mL. (n=3 biologically independent samples). (17B) The mean fluorescence intensity (MFI) of FITC-Ova in CD11c⁺ DCs at 24 h after treatment. FITC-Ova: 1.67 μg/mL; PIC: 4.67 μg/mL. (n=4 biologically independent samples). Data are shown as mean±SD. Statistical significance was calculated via one-way ANOVA test. *p<0.05, **p<0.01, ***p<0.001 and ****p<0.0001.

FIGS. 18A-18C (18A) Scheme for the co-culture of B16-SIINFEKL cells with splenocytes extracted from Ova, Ova/PIC, or Ova/CpG/ION injected mice. Quantification of CD44⁺, CD69⁺ and IFNγ⁺ cells out of (18B) CD4⁺CD3⁺CD45⁺ and (18C) CD8⁺CD3⁺CD45⁺ cells in splenocytes by flow cytometry. (n=8 biologically independent samples). Data are shown as mean±SD. Statistical significance was calculated via one-way ANOVA test. *p<0.05, **p<0.01, ***p<0.001 and ****p<0.0001. Gating strategy is shown in FIG. 19.

FIG. 19 Gating strategy of the T cell analysis as shown in FIGS. 18A-18C.

FIG. 20 IVIS images of B78 melanoma-bearing mice at indicated timepoints after Cy5-labeled PIC (Cy5-PIC) was intratumorally injected.

FIG. 21 Gating strategy for the analysis of Cy5-PIC internalization in antigen presenting cells in the tumor microenvironment and tumor-draining lymph nodes (TDLNs) after it was intratumorally injected into B78 melanoma-bearing mice.

FIGS. 22A-22C (22A) Scheme for the treatment. The tumor growth curves and average tumor volumes of B78 melanoma bearing mice at (22B) day 6 and (22C) day 14 after indicated treatments. The mice were euthanized at (22B) day 7 and (22C) day 15 for qPCR analysis of bulk tumor samples. (n=5 biologically independent animals). PIC: 140 μg/100 μL/dose. RT: 12 Gy. Data are shown as mean±SD. Statistical significance was calculated via one-way ANOVA test in 22B and 22C. *p<0.05, **p<0.01, ***p<0.001 and ****p<0.0001.

FIGS. 23A-23B (23A) Scheme for the study of the immunomodulatory effect of PIC+RT on the tumor microenvironment. (23B) The mRNA expression of Ifnβ1 and Mx1 in the B78 tumor microenvironment at day 7 after the indicated treatment. (n=5 biologically independent samples). PIC: 140 μg/100 μL/dose. RT: 12 Gy. Statistical significance was calculated via one-way ANOVA test in 23B and data are shown as mean±SD. *p<0.05, **p<0.01, ***p<0.001 and ****p<0.0001.

FIG. 24 The mRNA expression of Ifnβ1 and Mx1 in the B78 tumors at day 15 after PIC or control treatment. PIC: 140 μg/100 μL/dose. PIC was intratumorally injected at day 0, 3, 6 and 9. (n=5 biologically independent samples). Data are shown as mean±SD. Statistical significance was calculated via unpaired t-test. *p<0.05, **p<0.01, ***p<0.001 and ****p<0.0001.

FIGS. 25A-25B The growth of B78 melanoma tumors in syngeneic mice (25A—left) and average tumor volumes at day 14 (25B—right) after indicated treatments. The mice were euthanized at day 15 for flow cytometry analyses of tumors and TDLNs. PIC: 140 μg/100 μL/dose; RT: 12 Gy. (n=5 biologically independent animals). The indicated treatments were given per FIG. 4A. Data are shown as mean±SD. Statistical significance was calculated via one-way ANOVA test. *p<0.05, **p<0.01, ***p<0.001 and ****p<0.0001.

FIG. 26 Gating strategy for the analysis of innate immune cells in B78 tumors.

FIG. 27 Gating strategy for the analysis of adaptive immune cells in B78 tumors.

FIGS. 28A-28D (28A) Quantification and (28B) representative flow cytometry data identifying F4/80⁺ cells in CD11b CD45⁺ myeloid cells in B78 tumors after indicated treatments. (28C) Representative flow cytometry data identifying CD80⁺CD206⁻ cells (M1-like macrophages) and CD206⁺CD80⁻ cells (M2-like macrophages) in CD11b⁺F4/80⁺ macrophages in B78 tumors after indicated treatments. (28D) The mean fluorescence intensity (MFI) of CD80 and CD206, and their ratios relative to total CD11b⁺F4/80⁺ macrophages in B78 tumors after indicated treatments. PIC: 140 μg/100 μL/dose; RT: 12 Gy. (n=5 biologically independent samples). The indicated treatments were given per FIG. 4A and the tumor samples were collected at day 15 after initiation of indicated treatments. Statistical significance was calculated via one-way ANOVA test in 28A and 28D, and data are shown as mean±SD. *p<0.05, **p<0.01, ***p<0.001 and ****p<0.0001.

FIG. 29 The percentage of CD103⁺CD11b⁻ cDC1s and CD11b CD103⁻ cDC2s among CD11c⁺MHCII⁺ DCs in TDLNs from mice bearing B78 flank tumors, after indicated treatments. PIC: 140 μg/100 μL/dose; RT: 12 Gy. The indicated treatments were given per FIG. 4A and the tumor samples were collected at day 15 after initiation of indicated treatments.

FIGS. 30A-30B (30A) Quantification and (30B) representative flow cytometry data identifying CD3⁺ cells among CD45⁺ cells in B78 tumors after indicated treatments. PIC: 140 μg/100 μL/dose; RT: 12 Gy. (n=5 biologically independent samples). The indicated treatments were given per FIG. 4A and the tumor samples were collected at day 15 after initiation of indicated treatments. Statistical significance was calculated via one-way ANOVA test in 30A, and data are shown as mean±SD. *p<0.05, **p<0.01, ***p<0.001 and ****p<0.0001.

FIG. 31 Representative flow cytometry data quantifying CD25⁺FOXP3⁺ Tregs among CD4⁺CD3⁺CD45⁺ cells in B78 tumors after indicated treatments. PIC: 140 μg/100 μL/dose; RT: 12 Gy. The indicated treatments were given per FIG. 4A and the tumor samples were collected at day 15 after initiation of indicated treatments.

FIGS. 32A-32B The percentage of (32A) CD44⁺CD62L⁻CD4⁺ and (32B) CD44⁺CD62L⁻CD8⁺ effector memory cells out of CD45⁺CD3⁺ cells in B78 tumors after indicated treatment. PIC: 140 μg/100 μL/dose; RT: 12 Gy. (n=5 biologically independent samples). The indicated treatments were given per FIG. 4A and the tumor samples were collected at day 15 after initiation of indicated treatments. Data are shown as mean±SD. Statistical significance was calculated via one-way ANOVA test. *p<0.05, **p<0.01, ***p<0.001 and ****p<0.0001.

FIGS. 33A-33B The cell number ratios of (33A) CD4⁺ effector T cells: Tregs and (33B) CD8⁺ effector T cells: Tregs in B78 tumors after indicated treatments. CD4⁺ effector T cells: CD44⁺CD4⁺CD3⁺CD45⁺; CD8⁺ effector T cells: CD44⁺CD8⁺CD3⁺CD45⁺; Tregs: CD25⁺FOXP3⁺CD4⁺CD3⁺CD45⁺. PIC: 140 μg/100 μL/dose; RT: 12 Gy. (n=5 biologically independent samples). The indicated treatments were given per FIG. 4A and the tumor samples were collected at day 15 after initiation of indicated treatments. Data are shown as mean±SD. Statistical significance was calculated via one-way ANOVA test. *p<0.05, **p<0.01, ***p<0.001 and ****p<0.0001.

FIGS. 34A-34B The percentage of (34A) CD44⁺ and (34B) CD69⁺ cells out of CD3⁺CD4⁺ T cells (left) and CD3⁺CD8⁺ T cells (right) in TDLNs after indicated treatments. PIC: 140 μg/100 μL/dose; RT: 12 Gy. (n=5 biologically independent samples). The indicated treatments were given per FIG. 4A and the TDLNs were collected at day 15 after initiation of indicated treatments.

FIG. 35 Representative flow cytometry data of central memory T cells (CD44⁺CD62L⁺) and effector memory T cells (CD44⁺CD62L⁻) in CD4⁺CD3⁺CD45⁺ or CD8⁺CD3⁺CD45⁺ cells in TDLNs after indicated treatments. PIC: 140 μg/100 μL/dose; RT: 12 Gy. The indicated treatments were given per FIG. 4A and the TDLNs were collected at day 15 after initiation of indicated treatments.

FIGS. 36A-36B (36A) Scheme for the treatment of mice bearing a B78 melanoma and subsequent implantation with an unrelated Panc02 tumor. (36B) Average Panc02 tumor growth curves are shown after these tumors were engrafted in naïve control mice or in mice rendered disease-free from a B78 melanoma by PIC+RT+anti-CTLA-4. (control: n=5; tumor-free mice: n=4 biologically independent animals). (36C) Individual mouse tumor growth curves from (36B). Data are shown as mean±SD. Statistical significance was calculated via linear mixed effects modeling in 36B. *p<0.05, **p<0.01, ***p<0.001 and ****p<0.0001.

FIG. 37 TC11 tumor growth and mice survival after indicated treatments. RT: 12 Gy. Anti-CTLA-4:100 μg/100 μL/dose. (n=5 biologically independent animals). The indicated treatments were given per FIG. 6A. Data are shown as mean±SD. Statistical significance was calculated via linear mixed effects modeling and log-rank test for tumor growth and mice survival, respectively. *p<0.05, **p<0.01, ***p<0.001 and ****p<0.0001.

FIGS. 38A-38C (38A) Scheme for the treatment of mice bearing a B78 melanoma flank tumor. (38B) Average tumor growth curves of mice are displayed following the indicated treatment regimen. (n=8 biologically independent animals). (38C) Individual tumor growth curves for mice in (38B). RT: 12 Gy. PIC: 140 μg/100 μL/dose. C4 (anti-CTLA-4): 100 μg/100 μL/dose. Statistical significance was calculated via linear mixed effects modeling in 38B, and data are shown as mean±SD. *p<0.05, **p<0.01, ***p<0.001 and ****p<0.0001.

FIGS. 39A-39B (39A) Scheme for delivery of treatment for in vivo toxicity studies. (39B) No significant change was observed in the body weight of mice bearing a B78 melanoma after treatment with PIC+RT or PIC+RT+C4. PIC: 140 μg/100 μL/dose. RT: 12 Gy. C4 (anti-CTLA-4): 100 μg/100 μL/dose. (n=3 biologically independent animals). Data are shown as mean±SD. Statistical significance was calculated via one-way ANOVA test. *p<0.05, **p<0.01, ***p<0.001 and ****p<0.0001.

FIG. 40 The complete blood counts of B78 melanoma bearing mice after indicated treatments. PIC: 140 μg/100 μL/dose. RT: 12 Gy. C4 (anti-CTLA-4): 100 μg/100 μL/dose. (n=3 biologically independent samples). The indicated treatments were given per FIG. 39A. Data are shown as mean±SD. Statistical significance was calculated via one-way ANOVA test. *p<0.05, **p<0.01, ***p<0.001 and ****p<0.0001.

FIG. 41 The blood metabolic profiles of mice bearing a B78 melanoma after indicated treatments. PIC: 140 μg/100 μL/dose. RT: 12 Gy. C4 (anti-CTLA-4): 100 μg/100 μL/dose. (n=3 biologically independent samples). The indicated treatments were given per FIG. 39A. Data are shown as mean±SD. Statistical significance was calculated via one-way ANOVA test. *p<0.05, **p<0.01, ***p<0.001 and ****p<0.0001.

FIG. 42 Images of H&E stained sections of major organs (liver, spleen, kidney, intestine and femur) from mice bearing a B78 melanoma and treated with PIC+RT or PIC+RT+C4. PIC: 140 μg/100 μL/dose. RT: 12 Gy. C4 (anti-CTLA-4): 100 μg/100 μL/dose. The indicated treatments were given per FIG. 39A. A representative image of three independent samples from each group is shown.

FIGS. 43A-43K show PIC and IL2 increase the expression of pro-inflammatory cytokines and augment T cell activation. (43A) Particle size (left) and zeta potential (right) of PIC. (43B) Scheme of the studies (RT: 12Gy; PIC: 0.5 mg/mL; IL2: 67 IU/mL). The effects of PIC and IL2 on the (43C) Ifng and Il4 expression, (43D) Ifng: Il4 expression ratios, (43E) Fas expression, (43F) Nos2 and Arg1 expression, (43G) Nos2:Arg1 expression ratios, and (43H) Il1a expression of splenocytes in B78 co-cultures. (n=3 biologically independent samples). The percentages of CD69⁺ and CD44⁺ cells among (43I) CD4⁺ T cells (CD3⁺CD4⁺), (43J) CD8⁺ T cells (CD3⁺CD8⁻), and (43K) NK cells (NK1.1⁺CD3⁻) after indicated treatment in B78 co-cultures. (n=4 biologically independent samples). Statistical significance was calculated via one-way ANOVA test in 43C-43K. Data are presented as mean±SD. *p<0.05, **p<0.01, ***p<0.001 and ****p<0.0001. FIG. 43B was created with Biorender.com.

FIGS. 44A-44N illustrate that PIC enhanced the tumor therapeutic efficacy of RT+IL2 and induced an abscopal effect and an immune memory effect in B78 melanoma bearing mice. (44A) Scheme for the studies on B78 melanoma model. (44B) Average tumor growth curves and (44C) survival rate of mice after the indicated treatments. (PIC+RT+IL2: n=7; others: n=5 animals per cohort). (44D) Individual tumor growth curves in (44B). (44E) Average tumor growth curves after the naïve mice and disease-free mice (mice tumor-free from B78 melanoma after PIC+RT+IL2 treatment) were re-challenged with B78 melanoma cells. (Naïve mice: n=5; disease-free mice: n=7 animals per cohort). (44F) Individual tumor growth curves in (44E). (44G) Scheme for the studies on B78 melanoma two-tumor model. (44H) Average tumor growth curves after the indicated treatments. (44i) Individual tumor growth curves in (44H). (44J) Survival rate and (44K) body weight of mice after the indicated treatments. (RT+IL2: n=7; PIC+RT+IL2: n=8; others: n=5 animals per cohort). (44I) Scheme for the splenocytes co-culture studies. (44M) The percentages of CD69⁺ among CD4⁺ T cells and CD8⁺ T cells and the CD44 MFI on these T cells in the splenocytes in the indicated co-cultures. (44N) The relative expression of Ifnγ and Il2 in the splenocytes in the indicated co-cultures. (n=4 animals per cohort). CR: complete response. DF: Disease free mice. Statistical significance was calculated via linear mixed effects modeling in 44B, 44E and 44H, and log-rank test in 44C and 44J. Data are presented as mean±SD. *p<0.05, **p<0.01, ***p<0.001 and ****p<0.0001.

FIGS. 45A-45J illustrate PIC and IL2 can be delivered using a reduced number of injections and enhance response to anti-CTLA-4 checkpoint inhibition. (45A) Scheme for the injection regimens tested on B78 melanoma two-tumor bearing mice. (45B) Average tumor growth curves after the indicated treatments. (45C) Individual tumor growth curves in (45B). (45D) Survival rate and (45E) body weight of mice after the indicated treatments. (n=6 animals per cohort). (45F) Scheme for the studies of anti-CTLA-4 and (PIC+IL2)₀₊₆+RT combinations in B78 melanoma two-tumor bearing mice. (45G) Average tumor growth curves after the indicated treatments. (45H) Individual tumor growth curves in (45G). (45I) Survival rate and (45J) body weight of mice after the indicated treatments. (Control: n=5; others: n=6 animals per cohort). C4: anti-CTLA-4. Statistical significance was calculated via linear mixed effects modeling in 45B and 45G, and log-rank test in 45D and 45I. Data are presented as mean±SD. *p<0.05, **p<0.01, ***p<0.001 and ****p<0.0001.

FIGS. 46A-46N illustrate (PIC+IL2)₀₊₆+RT favorably immunomodulated the TME and the adjacent TDLNs. (46A) Scheme for the studies. (46B) The representative H&E and immunohistochemistry images of B78 melanomas (scale bar: 100 μm). (46C) The percentages of CD45⁺ immune cells among live cells in B78 melanomas. (46D) The percentages of CD80⁺CD206⁻ M1 macrophages and CD206⁺CD80⁻ M2 macrophages among CD11b⁺F4/80⁺ macrophages in B78 melanomas. (46E) The ratios of M1:M2 macrophages in B78 melanomas. (46F) The percentages of CD11c⁺MHCII⁺ DCs among CD45⁺ immune cells; the CD80 MFI on CD11c⁺MHCII⁺ DCs in B78 melanomas. (46G) The percentages of CD4⁺ T cells (CD4⁺CD3⁺) and CD8⁺ T cells (CD8⁺CD3⁺) among CD45⁺ immune cells, activated T cells (CD69⁺) and Tregs (CD25⁺FOXP3⁺) among CD4⁺ T cells and CD8⁺ T cells, and PD-1 MFI on CD8⁺ T cells in B78 melanomas. (46H) The percentages of NK1.1⁺CD3⁻ NK cells among CD45⁺ immune cells in B78 melanomas. (n=10 animals per cohort). (46I) The relative expression of Ifng, Il4 and their ratios in the bulk B78 tumor samples. (46J) The relative expression of Ifnb1 in the bulk tumor samples. (n=6 animals per cohort). (46K) The percentages of CD11b⁺F4/80⁺ macrophages among CD45⁺ immune cells, CD80⁺CD206⁻ M1 macrophages and CD206⁺CD80⁻ M2 macrophages among CD11b⁺F4/80⁺ macrophages, and the M1:M2 macrophages ratios in the right TDLNs. (46L) The percentages of CD11c⁺MHCII⁺ DCs among CD45⁺ immune cells in the right TDLNs. (n=6 animals per cohort). (46M) The percentages of activated (CD69⁺) T cells among CD4⁺ T cells and CD8⁺ T cells. (46N) The percentages of Tcm (CD44⁺CD62L⁻) and Tcm (CD44⁺CD62L⁺) among CD4⁺ T cells and CD8⁺ T cells. (n=10 animals per cohort). G1: control; G2: RT+IL2₀₊₆; G3: (PIC+IL2)₀₊₆+RT. Statistical significance was calculated via one-way ANOVA test in 46c-46N. Data are presented as mean±SD. *p<0.05, **p<0.01, ***p<0.001 and ****p<0.0001.

FIGS. 47A-47H illustrate (PIC+IL2)₀₊₆+RT treatment activated systemic anti-tumor T cell immunity and immunomodulated the distant tumors that were not treated directly. (47A) The percentages of CD8⁺CD3⁺ cells among CD45⁺ immune cells, the CD44 MFI on CD8⁺ T cells, and the percentages of NKG2D⁺ cells among CD8⁺ T cells in the blood. (47B) The percentages of CD4⁺CD3⁺ cells among CD45⁺ immune cells, the CD44 MFI on CD4⁺ T cells, and the percentages of NKG2D⁺ cells among CD4⁺ T cells in the blood. (n=6 animals per cohort). (47C) The percentages of CD45⁺ immune cells among live cells in distant B78 melanomas. (47D) The percentages of CD4⁺CD3⁺ cells and CD8⁺CD3⁺ cells among CD45⁺ immune cells in distant melanomas. (47E) The representative H&E and immunohistochemistry images of distant B78 melanomas (scale bar: 100 μm). (47F) The percentages of CD69⁺ cells among CD8⁺ T cells, and the PD-1 MFI and CD44 MFI on the CD8⁺ T cells in distant B78 melanomas. (47G) The percentages of CD80⁺CD206⁻ M1 macrophages and CD206⁺CD80⁻ M2 macrophages among CD11b⁺F4/80⁺ macrophages, and the M1:M2 macrophage ratios in distant B78 melanomas. (47H) The percentages of CD11c⁺MHCII⁺ DCs among CD45⁺ immune cells; the CD80 MFI on CD11c⁺MHCII⁺ DCs in distant B78 melanomas. (n=10 animals per cohort). (47F) G1: control; G2: RT+IL2₀₊₆; G3: (PIC+IL2)₀₊₆+RT. Statistical significance was calculated via one-way ANOVA test in 47A-47E and 47G. Data are presented as mean±SD. *p<0.05, **p<0.01, ***p<0.001 and ****p<0.0001.

FIGS. 48A-48D illustrate (PIC+IL2)₀₊₆+RT treatment delayed the tumor growth of MOC2 head and neck tumors, increased mouse survival, and improved the responsiveness of MOC2 tumors to anti-CTLA-4. (48A) Scheme for the studies. (48B) Average tumor growth curves and (48C) mouse survival after the treatments indicated. (48D) Individual tumor growth curves in (48B). (n=6 animals per cohort) . . . . Statistical significance was calculated via linear mixed effects modeling in 48B, and log-rank test in 48c. Data are presented as mean±SD. *p<0.05, **p<0.01, ***p<0.001 and ****p<0.0001.

FIG. 49 illustrates the proposed mechanisms of action for PIC+RT+IL2 in tumor treatment. RT: RT can induce the immunogenic cell death of cancer cells and improve the susceptibility of cancer cells to immune-mediated killing, while also increasing the tumor-infiltrating by immunosuppressive Tregs and M2 macrophages. IL2 injection: when IL2 is injected into a radiated tumors, it can promote the clonal expansion and activation of effector T cells but also does this for Tregs, which can often exhibit increased infiltration in the irradiated TME. PIC injection: when PIC is injected to the radiated tumors, it can relieve immunosuppression by decreasing tumor infiltration by M2 macrophages and Tregs, but its ability to prime and clonally expand T cells is limited. PIC+IL2 injection: when PIC+IL2 is injected to the radiated tumors, PIC can dampen the immunosuppression caused by RT by decreasing tumor infiltration by Tregs and M2 macrophages; PIC combined with IL2 promotes the proliferation and functions of effector T cells and prime systemic anti-tumor T cell immunity to eradicate distant tumors. This figure was created with Biorender.com.

FIG. 50 shows the gating strategy for the flow cytometry analyses of co-culture studies in vitro.

FIGS. 51A-51B show the percentage of CD25⁺FOXP3⁺ Tregs among (51A) CD4 T cells and (51B) CD8 T cells after different treatment in B78 co-cultures. (n=4 biologically independent samples). The treatment was performed per FIG. 43B. Statistical significance was calculated via one-way ANOVA test. Data are presented as mean±SD. *p<0.05, **p<0.01, ***p<0.001 and ****p<0.0001.

FIGS. 52A-52B show the percentages of PD-1⁺ cells among CD4 T cells (52A) and CD8 T cells (52B) after different treatment in B78 co-cultures. (n=4 biologically independent samples). The treatment was performed per FIG. 43B. Statistical significance was calculated via one-way ANOVA test. Data are presented as mean±SD. *p<0.05, **p<0.01, ***p<0.001 and ****p<0.0001.

FIG. 53 shows the effects of PIC, IL2 and PIC+IL2 on the gene expression of Ifnγ, Il4, Fas, Nos2, Arg1 and Il1α by splenocytes. The splenocytes were treated with PIC (0.5 μg/mL) or/and IL2 (67 IU/mL), and analyzed by RT-qPCR 24 hours later. (n=4 biologically independent samples). Statistical significance was calculated via one-way ANOVA test. Data are presented as mean±SD. *p<0.05, **p<0.01, ***p<0.001 and ****p<0.0001.

FIG. 54 shows the effects of PIC, IL2 and PIC+IL2 on the activation state (CD69⁺) and effector state (CD44⁺) of CD4 T cells, CD8 T cells and NK cells. The splenocytes were treated with PIC (0.5 μg/mL) or/and IL2 (67 IU/mL), and analyzed by flow cytometry 24 hours later. (n=4 biologically independent samples). Statistical significance was calculated via one-way ANOVA test. Data are presented as mean±SD. *p<0.05, **p<0.01, ***p<0.001 and ****p<0.0001.

FIG. 55 shows the gating strategy for the flow cytometry analyses of the immune memory effects.

FIGS. 56A-56C shows PIC injection at day 0 exhibited to be important in the PIC+RT+C4 treatment regimens. (56A) Scheme for the studies. (56B) Average tumor growth curves after the treatments indicated. (Control and PIC+RT+C4: n=5; others: n=4 biologically independent mice). (56C) Individual tumor growth curves in (56B).

FIG. 57 shows the gating strategy for the innate immune cells in the tumor tissues.

FIG. 58 shows the gating strategy for the adaptive immune cells in the tumor tissues.

FIG. 59 shows the gating strategy for the innate immune cells in the TDLNs.

FIG. 60 shows the gating strategy for the adaptive immune cells in the TDLNs.

FIGS. 61A-61B shows the tumor growth curves of B78 melanoma two-tumor mice after the indicated treatment for primary (61A) and distant (61B) tumors. The mice were euthanized at day 15 after the initiation of treatment and the tumor tissues and tumor-draining lymph nodes were analyzed with flow cytometry. (n=10 biologically independent animals). The treatment was performed per FIG. 46A.

FIGS. 62A-62F shows (PIC+IL2)₀₊₆+RT treatment immunomodulated B78 melanomas and the adjacent TDLNs. (62A) The percentages of CD11b⁺F4/80⁺ macrophages among CD45⁺ immune cells in B78 melanomas. (62B) The CD80 MFI and CD206 MFI on CD11b⁺F4/80⁺ macrophages and their expression ratios in B78 melanomas. (62C) The CD44 MFI on CD4 T cells and CD8 T cells in B78 melanomas. (62D) The PD-1 MFI on CD4 T cells in B78 melanomas. (62E) The percentages of CD69⁺ cells among NK1.1⁺CD3⁻ NK cells and the CD44 MFI on NK cells in B78 melanomas. (n=10 biologically independent animals). (62F) The CD80 MFI and CD206 MFI on CD11b⁺F4/80⁺ macrophages and their ratios in the TDLNs. (n=6 biologically independent animals). The treatments were performed per FIG. 46A. G1: control; G2: IL2₀₊₆+RT; G3: (PIC+IL2)₀₊₆+RT. Statistical significance was calculated via one-way ANOVA test. Data are presented as mean±SD. *p<0.05, **p<0.01, ***p<0.001 and ****p<0.0001.

FIGS. 63A-63B shows (PIC+IL2)₀₊₆+RT treatment regulated the immune cytokines gene expression in both the primary and distant B78 melanomas. (63A) The relative expression of Il1α and Fas in primary B78 melanomas. (63B) The relative expression of Ifnγ, Il4, Ilβ1, Il1α, Fas, and the expression ratios of Ifnγ. Il4 in distant B78 melanomas. (n=6 biologically independent animals). The treatments were performed per FIG. 46A. G1: control; G2: IL2₀₊₆+RT; G3: (PIC+IL2)₀₊₆+RT. Statistical significance was calculated via one-way ANOVA test. Data are presented as mean±SD. *p<0.05, **p<0.01, ***p<0.001 and ****p<0.0001.

FIG. 64 shows the representative H&E images of the B78 melanoma two-tumor bearing mice after the treatment indicated. The treatments were performed per FIG. 46A. (n=3 biologically independent animals).

FIGS. 65A-65B shows (PIC+IL2)₀₊₆+RT treatment regulated the immune microenvironment of distant B78 melanomas but exhibited negligible effects on the distant lymph nodes. (65A) The percentages of CD11b⁺F4/80⁺ macrophages among CD45⁺ immune cells in B78 melanomas; the CD80 MFI and CD206 MFI on CD11b⁺F4/80⁺ macrophages and their expression ratios in B78 melanomas. (n=10 biologically independent animals). (65B) The analysis of macrophages, dendritic cells and T cells in the distant lymph nodes. (n=6 biologically independent animals for innate immune cells; n=10 biologically independent animals for adaptive immune cells). The treatments were performed per FIG. 46A. G1: control; G2: IL2₀₊₆+RT; G3: (PIC+IL2)₀₊₆+RT. Statistical significance was calculated via one-way ANOVA test. Data are presented as mean±SD. *p<0.05, **p<0.01, ***p<0.001 and ****p<0.0001.

FIGS. 66A-66B show the biodistribution of Cy5-labeled PIC in the primary/distant B78 melanomas (66A) and primary/distant TDLNs (66B). PIC was intratumorally injected into the primary tumors of B78 two-tumor bearing mice at day 0 and day 6 with 560 μg PIC/injection. The imaging was taken at day 15. (n=3 biologically independent animals).

FIG. 67 shows the gating strategy for the analysis of T cells in the blood.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The following terms are used throughout as defined below. All other terms and phrases used herein have their ordinary meanings as one of skill in the art would understand.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

“Molecular weight” as used herein with respect to polymers refers to number-average molecular weights (Mₙ) and can be determined by techniques well known in the art including gel permeation chromatography (GPC). GPC analysis can be performed, for example, on a D6000M column calibrated with poly(methyl methacrylate) (PMMA) using triple detectors including a refractive index (RI) detector, a viscometer detector, and a light scattering detector, and N,N′-dimethylformamide (DMF) as the eluent. “Molecular weight” in reference to small molecules and not polymers is actual molecular weight, not number-average molecular weight.

A “dye” refers to small organic molecules having a molecular weight (actual, not number average) of 2,000 Da or less or a protein which is able to emit light. Non-limiting examples of dyes include fluorophores, chemiluminescent or phosphorescent entities. For example, dyes useful in the present technology include but are not limited to cyanine dyes (e.g., Cy2, Cy3, Cy5, Cy5.5, Cy7, and sulfonated versions thereof), fluorescein isothiocyanate (FITC), ALEXA FLUOR® dyes (e.g., ALEXA FLUOR® 488, 546, or 633), DYLIGHT® dyes (e.g., DYLIGHT® 350, 405, 488, 550, 594, 633, 650, 680, 755, or 800) or fluorescent proteins such as GFP (Green Fluorescent Protein).

The present technology provides nanoparticles (also referred to herein as “PIC NP”) that can potentiate the effect of RT on ICB and/or immune adjuvants therapy. In a first aspect, each nanoparticle includes polylysine electrostatically bound to iron oxide nanoparticles (also referred to herein as “ION”) and CpG oligodeoxynucleotide.

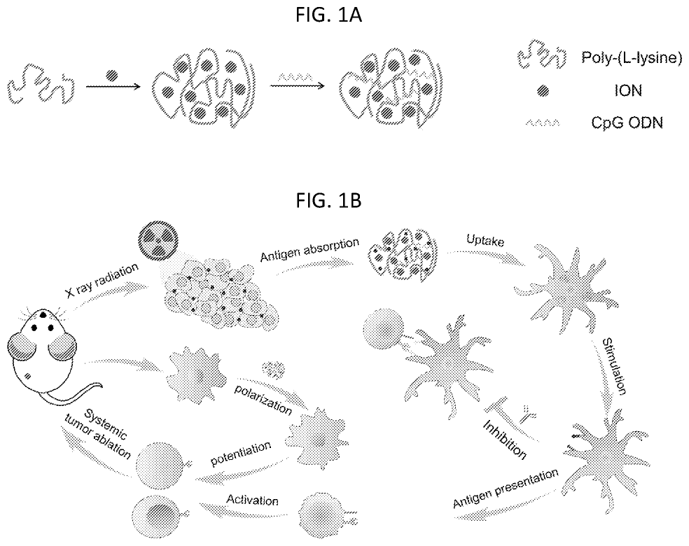

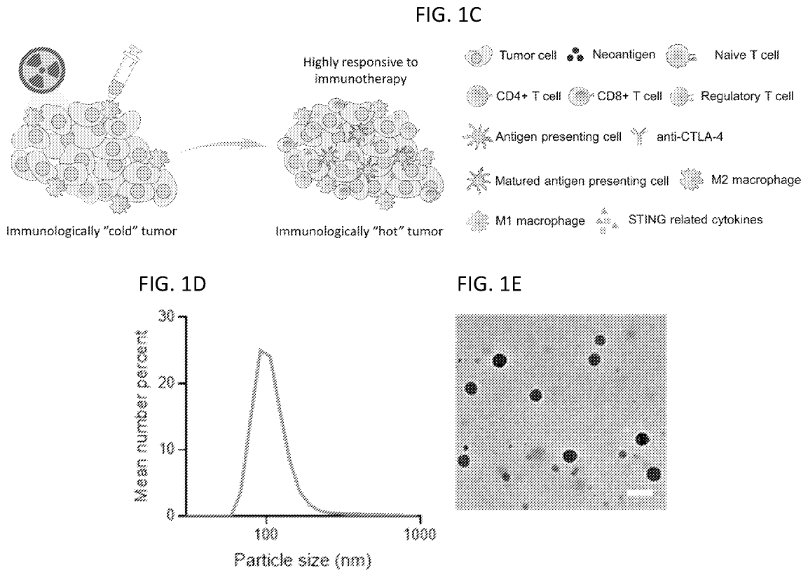

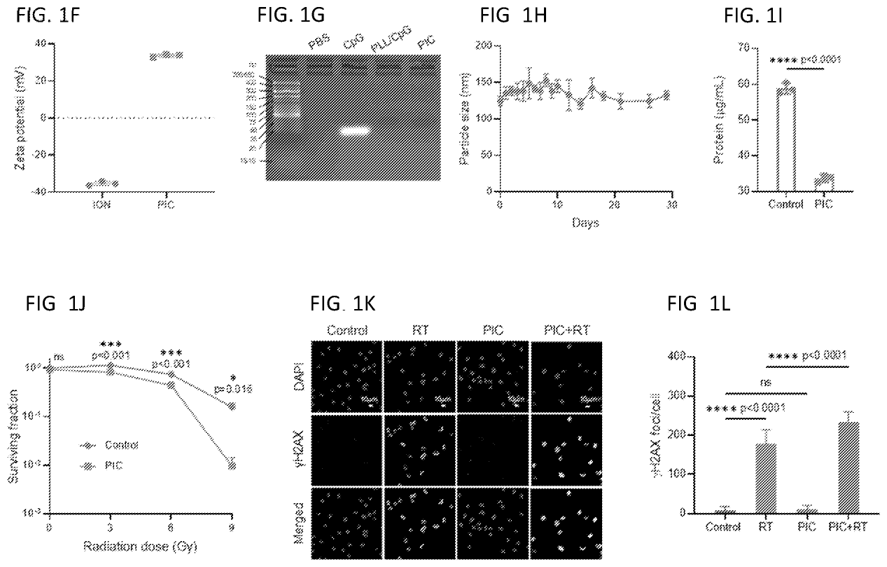

While not wishing to be bound by theory, the positively charged PIC NP of the present technology was designed to capture tumor associated antigens (TAAs) released from dying radiated cells via electrostatic interaction. The PIC NP facilitates the uptake of these TAAs by antigen presenting cells (APCs) via endocytosis. CpG, as a Toll-like receptor-9 (TLR-9) agonist, was included to stimulate the maturation of APCs so that the TAAs would be optimally degraded, processed into peptides, and presented on major histocompatibility complex I (MHC I) and major histocompatibility complex II (MHC II) at the APC plasma membrane to activate CD8+ and CD4+ T cell responses, respectively. In addition, the ION component is believed to reduce the known effect of RT in stimulating tumor infiltration by M2 polarized macrophages, specifically immuno-modulating the radiated tumor microenvironments by favoring tumor infiltration with pro-inflammatory M1 macrophages (FIG. 1B). By diminishing M2 macrophage tumor infiltration, increasing tumor infiltration by M1 macrophages, activating APCs, enhancing TAA uptake by APCs, accentuating RT-mediated activation of a type I interferon response downstream of cGAS/STING, and thereby priming a more effective tumor-specific T cell response, PIC NP in conjunction with RT renders immunologically “cold” tumors more responsive to ICB (FIG. 1C).

The present nanoparticles include iron oxide nanoparticles. Iron oxide nanoparticles that remain charged at physiological pH may be used in the present technology. In any embodiments, the iron oxide nanoparticles may be negatively charged. In any embodiments, the iron oxide nanoparticles may be selected from one or more of Fe₃O₄, FeO, iron (II,III) oxide, or Fe₂O₃. or their mixtures. The iron oxide nanoparticles may have a range of hydrodynamic diameters, e.g., 1 nm to 50 nm, and may diameters of 1, 2, 3, 4, 5, 6, 7, 8, 9, 10, 15, 20, 25, 30, 35, 40, 45, or 50 nm, or a range between and including any two of the foregoing values.

In PIC NP of the present technology, iron oxide nanoparticles (“ION”) may make up from about 30 wt % to about 80 wt % of the PIC NP. Thus, in any embodiments, the ION may make up about 30, 35, 40, 45, 50, 55, 60, 65, 70, 75, or 80 wt % of the PIC NP or a range between and including any two of the fore going values. For example, in any embodiments, the ION may make up from about 45 wt % to about 70 wt % or from about 45 wt % to about 55 wt % of the PIC NP.

PIC NP of the present technology include polylysine (also referred to as PLL herein). The polylysine may make up about 5 wt % to about 50 wt % of the PIC NP, e.g., 5, 10, 15, 20, 25, 30, 35, 40, 45, or 50 wt % or a range between and including any two of the foregoing values. For example, in any embodiments the PIC NP may include about 10 wt % to about 40 wt %, or about 30 wt % to about 45 wt % PLL. The PLL may be, e.g., α-PLL (such as DL-polylysine, L-polylysine, or D-polylysine) or ε-PLL. The polylysine may have a molecular weight of about 5 kDa to about 150 kDa, e.g., about 5, 10, 15, 20, 30, 35, 40, 45, 50, 55, 60, 65, 70, 80, 90, 100, 110, 120, 130, 140, or 150 kDa or a range between and including any two of the foregoing values. For example, in any embodiments, the PIC NP may include PLL having a molecular weight of about 30 kDa to about 70 kDa. The PLL can also be replaced by other positively charged polymers having a plurality of positively charged groups, such as linear polyethyleneimine, hyperbranched polyethyleneimine, polyarginine, chitosan, and their derivatives. These polymers may also have a molecular weight of about 5 kDa to about 150 kDa, e.g., about 5, 10, 15, 20, 30, 35, 40, 45, 50, 55, 60, 65, 70, 80, 90, 100, 110, 120, 130, 140, or 150 kDa or a range between and including any two of the foregoing values. Thus, in some embodiments, the nanoparticle includes a positively charged polymer of the foregoing molecular weight ranges electrostatically bound to ION and CpG oligodeoxynucleotide. In some such embodiments the positively charged polymer has a molecular weight of about 30 kDa to about 70 kDa.

PIC NP of the present technology include one or more CpG oligodeoxynucleotides (also referred to as “CpG ODN” herein). CpG ODN are short (i.e., 4-50 residues, e.g., 4, 5, 6, 7, 8, 9, 10, 15, 20, 25, 30, 35, 40, 45 or 50 residues, or a range between and including any two of the foregoing values, e.g., 10-30 residue) oligodoxynucleotides having one or more, unmethylated cytosine-guanine dinucleotide motifs (referred to herein as the CpG motif) that are immunostimulatory and conserve as a ligand for a Toll-like family signaling molecule such as Toll-Like Receptor 9 (TLR9). In some embodiments, the CpG ODN can contain more than one CpG dinucleotide, arranged either contiguously or separated by intervening nucleotide(s) The CpG motif(s) can be in the interior of the oligonucleotide sequence. The phosphodiester linkage in CpG ODN may be replaced in part or in whole by nuclease-resistant linkages known in the art, such as phosphorothioate linkages. Numerous nucleotide sequences of CpG ODN stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, A M, Advanced drug delivery reviews 61(3):195-204 (2009), incorporated herein by reference). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, G L, PNAS USA 94(20): 10833-7 (1997); Dalpke, A H, Immunology 106(1): 102-12 (2002); Hartmann, G, J of Immun. 164(3): 1617-2 (2000), each of which is incorporated herein by reference).

A variety of CpG ODN may be used in the present PIC NP, including e.g., CpG ODN 1826, CpG ODN 1668, CpG ODN M362, CpG ODN 2336, CpG ODN 2007, CpG ODN 2006, CpG ODN 2216, CpG ODN 1585, CpG ODN 1018, CpG ODN 2395, CpG ODN BW006, CpG ODN D-SL01, CpG ODN D-SL03.

The CpG ODN may make up about 5 wt % to about 30 wt % of the PIC NP, e.g., about 5, 6, 7, 8, 9, 10, 15, 20, 25, or 30 wt % or a range between and including any two of the foregoing values. For example, in any embodiments the CpG oligodeoxynucleotide may make up about 5 wt % to about 15 wt % of the PIC NP or about 6 wt % to about 20 wt %.

In any embodiments, the present PIC NP may further include a label. For example, the PIC NP may include a label selected from the group consisting of dyes, radioisotope chelators for PET imaging, and chelators for MRI imaging. The label may be attached to the PIC NP by any suitable method known in the art. For example, the label may be conjugated to at least a portion of PLL used in the PIC NP. In any embodiments, a fluorescent lable may be conjugated to the PLL via an amide, ester, or urethane group.

In the present technology, the surface of the PIC NP may be charged (measured as zeta potential), e.g., +10 mV to +60 mV. Nanoparticle surface potential (i.e., zeta potential) may be measured by DLS in an applied electric field at any suitable voltage (e.g., 40 V; the measured surface potential will be independent of the exact voltage used) at 0.1 mg/mL, pH 7.4, 25° C. Examples of the surface potential of the present NPs include +10, +15, +20, +25, +30, +35, +40, +45, +50, +55, or +60 mV, or a range between and including any two of the foregoing values. For example, the zeta potential may be +25 to +35 mV.

The present NPs may be roughly sphere-shaped or may have a more elongated shape. Nevertheless, the “average diameter” of the present PIC NP means the average hydrodynamic diameter and may range from 50 nm to 500 nm. Thus, the present PIC NP may have an average hydrodynamic diameter of 50, 60, 70, 80, 90, 100, 110, 125, 150, 175, 200, 250, 300, 400, or 500 nm or a range between and including any two of the foregoing values. In any embodiments herein, they may have an average hydrodynamic diameter of 50 to 250 nm, 50 to 200 nm, 60 nm to 200 nm, or 80 nm to 200 nm.

The present PIC NP may be stored in aqueous solutions or suspensions at −20° C. in the presence of any suitable cryoprotectant. In any embodiments, the aqueous storage solution includes a non-sugar polyol of 2, 3, 4, 5 or 6 carbons, optionally with a suitable buffer. Thus, in any embodiments, the aqueous storage solution may include, e.g., glycerol, ethylene glycol, glycerol+HEPES, ethylene glycol+HEPES, glycerol+ethylene glycol, or glycerol+ethylene glycol+HEPES. The concentration of glycerol or ethylene glycol may be about 5 wt % to about 50 wt %. Thus, PIC NP may be stored at −20° C. in the presence of about 5 wt %, about 10 wt %, about 15 wt %, about 20 wt %, about 25 wt %, about 30 wt %, about 35 wt %, about 40 wt %, about 45 wt %, or about 50 wt % glycerol or ethylene glycol, or a range between and including any two of the foregoing values. The concentration of buffer, e.g., HEPES, may be about 5 to about 100 mM. Thus, about 5, about 10, about 15, about 20, about 25, about 30, about 35, about 40, about 45, about 50, about 55, about 60, about 65, about 70, about 75, about 80, about 85, about 90, about 95, or about 100 mM or a range between and including any two of the foregoing values may be added when PIC NP aqueous solutions or suspensions are stored at −20° C.

The present NPs may be lyophilized in the presence of saccharide cryoprotectants without their physical properties being influenced, i.e., without damage when stored at low temperatures, especially temperatures below freezing. Saccharide cryoprotectants of the present technology include mono- and disaccharides. The cryoprotectants may, e.g., be sucrose, mannitol, trehalose, lactose, maltose, or cellobiose. The concentration of the cryoprotectant in an aqueous storage solution or lyophilization solution may be about 0.3 wt % to 30 wt %. Thus, 0.3 wt %, 0.5 wt %, 1 wt %, 2 wt %, 5 wt %, 10 wt %, 15 wt %, 20 wt %, 25 wt %, or 30 wt % cryoprotectant or a range between and including any two of the foregoing values.

In other aspects, there are provided methods of use for the PIC NP. In one aspect the present technology provides methods of sensitizing tumor cells to radiation therapy and/or creating radiation dose heterogeneity in a radiated tumor microenvironment comprising administering an effective amount of any PIC NP disclosed herein to the tumor cells. In another aspect, there are provided methods of stimulating antigen presenting cells comprising administering an effective amount of any PIC NP disclosed herein to the tumor cells. In another aspect, there are provided methods of increasing M1:M2 macrophages ratios at a tumor site comprising administering an effective amount of any PIC NP disclosed herein to the tumor cells. In still another aspect, the present technology provides methods of enhancing stimulation of a type I interferon response comprising administering an effective amount of any PIC NP disclosed herein to the tumor cells.

In another aspect, the present technology provides methods of treatment comprising administering to a subject suffering from cancer an effective amount of any PIC NP disclosed herein and an effective amount of radiation therapy. In some embodiments, the treatment increases the expression of pro-inflammatory cytokines. In some embodiments, the treatment increases T cell activation. In any embodiments, the cancer may be an immunologically hot cancer. In any embodiments, the cancer may be an immunologically cold cancer. Immunologically hot cancers or tumors refers to cancers (or tumors) that exhibit a measurable response to ICB, whereas immunologically cold cancers or tumors do not exhibit a measurable response. The method may further include administering to the subject an effective amount of a checkpoint inhibitor and/or an immune adjuvant. The subject may be a human or animal subject (e.g., a mammal such as a mouse, rat, dog, cat, pig, horse, cow, monkey or the like). In any embodiments of the method, the subject may be a human. A number of cancers are known to be immunologically hot or immunologically cold with respect to various ICB therapies. Even when a cancer is immunologically hot, use of the present PIC-NP may increase the depth and duration of response to RT and ICB over those therapies in the absence of PIC-NP. Hence, in any embodiments, the cancer may be selected from the group consisting of breast cancer, bladder cancer, cervical cancer, colon cancer, head and neck cancer, liver cancer, lung cancer, pancreatic cancer, prostate cancer, renal cell cancer, melanoma, sarcomas, stomach cancer, rectal cancer, and Hodgkin lymphoma. In any embodiments of the method, the cancer may be breast cancer, prostate cancer or melanoma. In any embodiments, the checkpoint inhibitor may be a CTLA4 inhibitor, PD1 inhibitor, PDL1 inhibitor, PDL2 inhibitor, or combination thereof. In any embodiments, the immune adjuvant may be a cytokine adjuvant. In any embodiments, the immune adjuvant may be IL-2, MPLA, IL-15, poly(I:C), poly(A:U), resiquimod, imiquimod, aluminum hydroxide, aluminum phosphate, cGAMP, a derivative of one of the foregoing, or a combination of any two or more thereof.

The compositions described herein can be formulated for various routes of administration, for example, by parenteral, intravitreal, intrathecal, intracerebroventricular, rectal, nasal, vaginal administration, direct injection into the target organ, or via implanted reservoir. Parenteral or systemic administration includes, but is not limited to, subcutaneous, intravenous, intraperitoneal, and intramuscular injections. The following dosage forms are given by way of example and should not be construed as limiting the instant present technology.

Injectable dosage forms generally include solutions or aqueous suspensions which may be prepared using a suitable dispersant or wetting agent and a suspending agent so long as such agents do not degrade the PIC NP described herein. Injectable forms may be prepared with acceptable solvents or vehicles including, but not limited to sterilized water, phosphate buffer solution, Ringer's solution, 5% dextrose, or an isotonic aqueous saline solution.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference. Exemplary carriers and excipients may include but are not limited to USP sterile water, saline, buffers (e.g., phosphate, bicarbonate, etc.), tonicity agents (e.g., glycerol).

Specific dosages of the present NPs may be adjusted depending on conditions of disease, the age, body weight, general health conditions, sex, and diet of the subject, dose intervals, administration routes, excretion rate, and combinations and amounts of drugs and radiation used. Any of the above dosage forms containing effective amounts are within the bounds of routine experimentation and therefore, within the scope of the present technology. By way of example only, such dosages may be used to administer effective amounts of the present PIC NP to the patient and may include 0.1, 0.2, 0.3, 0.4, 0.5, 0.75, 1.0, 1.5, 2.0, 2.5, 3.0, 4.0, 5.0, 6.0, 7.0, 8.0, 9.0, 10, 11, 12, 13, 14, 15, 20, 25, 30, 40, or 50 mg/kg or a range between and including any two of the forgoing values such as 0.1 to 15 mg/kg, or 2 to 8 mg/kg or even 4 to 6 mg/kg. Such amounts may be administered parenterally (including but not limited to direct injection into the solid tumor) as described herein and may take place over a period of time including but not limited to 5 minutes, 10 minutes, 20 minutes, 30 minutes, 45 minutes, 1 hour, 2 hours, 3 hours, 5 hours, 10 hours, 12, hours, 15 hours, 20 hours, 24 hours or a range between and including any of the foregoing values. The frequency of administration may vary, for example, once per day, per 2 days, per 3 days, per week, per 10 days, per 2 weeks, or a range between and including any of the foregoing frequencies. Alternatively, the compositions may be administered once per day on 2, 3, 4, 5, 6 or 7 consecutive days. A complete regimen may thus be completed in only a few days or over the course of 1, 2, 3, 4 or more weeks.

The present methods of cancer treatment employ radiation therapy in conjunction with delivery of PIC NP and subsequently, ICB and/or immune adjuvants. In some aspects of the present methods, an effective amount of radiation therapy can be administered to the site(s) of the breast cancer, bladder cancer, cervical cancer, colon cancer, head and neck cancer, liver cancer, lung cancer, pancreatic cancer, prostate cancer, renal cell cancer, melanoma, sarcomas, stomach cancer, rectal cancer, and Hodgkin lymphoma. In any embodiments of the method, the cancer may be breast cancer, prostate cancer or melanoma. In any embodiments of the present methods, total dosages of radiation may range, e.g., from 2 Gy to 70 Gy, and are typically (but need not be for lower doses) fractioned into multiple doses. For example, the dosages may be in the range of about 1 unit (Grays) per day (Gy/day) to about 34 Gy/day. Dosages of radiation therapy can also vary from about 1 Gy/day to about 5 Gy/day or about 1 Gy/day to about 3 Gy/day. Examples of suitable radiation therapy dosages include, but are not limited to about 1, 2, 3, 4, 5, 6, 7, and 8 Gy/day or a range between or including any of the individual values described herein. Specific dosages of an effective amount of radiation in conjunction with PIC NP and ICB and/or immune adjuvants in the present methods may be adjusted depending on conditions of disease, the age, body weight, general health conditions, sex, and diet of the subject, dose intervals, administration routes, excretion rate, and combinations and amounts of drugs used. Any of the above dosage forms containing effective amounts are well within the bounds of routine experimentation and therefore, well within the scope of the present technology.

In some embodiments, the present methods can include administering to cancer cells (e.g., breast, bladder, cervical, colon, head and neck, liver, lung, pancreatic, prostate, renal cell, melanoma, sarcoma, stomach, rectal, and Hodgkin lymphoma cancer cells) radiation therapy over a period of time of about 1 to about 5 days, overlapping with or in sequence with administration of PIC NP and ICB and/or immune adjuvants therapy. For example, radiation therapy may be administered to an of the foregoing cancer cells from about 1 to about 5 days following pretreatment with the PIC NP. Examples of suitable time periods for radiation therapy include, but are not limited to, about 1 day or about 1 to about 2, about 3, about 4, or about 5 days. The present methods can also include administering to cancer cells radiation therapy over a period of time simultaneously with or separately from administration of PIC NP or PIC NP and ICB or PIC NP and immune adjuvants or PIC NP and ICB and immune adjuvants. For example, radiation therapy can be administered to cancer cells at the same time (e.g., same day) as PIC NP. PIC NP may also be administered prior to and after RT, e.g., PIC NP may be administered the day before and the day after administration of radiation.

In some embodiments, multiple rounds of radiation doses in conjunction with PIC NP and ICB and/or immune adjuvants therapy may be administered. For example, 2, 3, 4, 5, 6, 7, 8, 9, or even 10 rounds of the radiation in conjunction with single or multiple doses of PIC NP and ICB and/or immune adjuvants may be administered, with days or weeks or even months in between each round. In some embodiments, the rounds of radiation compositions are administered 4, 5, 6, 7, 8, 9, or 10 days apart, and in others the rounds are administered 1, 2, 3, 4, 5, 6, 7, or 8 weeks apart, or even 1, 2, 3, 4, 5, or 6 months apart. In any embodiments, rounds of PIC NP and ICB may precede, accompany, or follow the rounds of radiation. Selecting the number of rounds and the interval(s) between each round is within the skill in the art and the bounds of routine experimentation and therefore, well within the scope of the present technology.

In some aspects, the present methods can include in vitro administration to cancer cells (e.g., breast, bladder, cervical, colon, head and neck, liver, lung, pancreatic, prostate, renal cell, melanoma, sarcoma, stomach, rectal, and Hodgkin lymphoma cancer cells) of an effective amount of radiation therapy in conjunction with the PIC NP described herein. For example, the present methods can include administering to cancer cells harvested from laboratory culture, or any similar in vitro cell growth procedure known to a person having ordinary skill in the art, an effective amount of radiation therapy in conjunction with PIC NP. Additionally, an effective amount of radiation therapy can be administered in vitro to cancer cells sequentially, simultaneously, or separately at any of the dosage ranges described herein in conjunction with PIC NP.

In some aspects, the present methods include administering to cancer cells an effective amount of radiation therapy in conjunction with PIC NP. In some embodiments, the cancer cells are in a subject, such as a human or animal subject (e.g., mouse, rat, dog, cat, pig, horse, cow, monkey or the like). In certain embodiments, the PIC NP comprises an aqueous carrier and is administered to the subject intravenously or intraperitoneally (e.g., directly to the cancer site in the animal body). The aqueous carriers can include saline or an aqueous carbohydrate solution, for example, 0.9% NaCl solution or a 5% aqueous saccharide solution. The aqueous saccharide solution can be, for example, dextrose or glucose. The aqueous carrier can also be any sterile aqueous solutions of water-soluble salts, for example, NaCl. The aqueous solutions can also be isotonic. The aqueous solutions may be suitably buffered. Aqueous solutions, described herein, can be suitable for intravenous, intramuscular, subcutaneous, intraperitoneal, and intratumoral injection. Appropriate sterile aqueous media can be purchased or can be prepared by standard techniques well known to those skilled in the art.

In any embodiments, dosages for an immune checkpoint inhibitor and/or an immune adjuvant as described herein may be determined empirically in individuals who have been administered one or more doses of the immune checkpoint inhibitor or the immune adjuvant. Individuals may be administered incremental dosages of the immune checkpoint inhibitor and/or immune adjuvant. To assess efficacy of an administered immune checkpoint inhibitor and/or immune adjuvant, one or more aspects of a cancer (e.g., tumor formation or tumor growth) may be analyzed. Specific dosages of the checkpoint inhibitors and/or immune adjuvants may be adjusted depending on conditions of disease, the age, body weight, general health conditions, sex, and diet of the subject, dose intervals, administration routes, excretion rate, and combinations and amounts of drugs & radiation used.

Generally, for administration of any of checkpoint inhibitor and/or immune adjuvant, a daily dosage might range from about 0.1 μg/kg to about 100 mg/kg, e.g., 0.1 μg/kg, 0.3 μg/kg, 0.5 μg/kg, 0.7 μg/kg, 1 μg/kg, 1.5 μg/kg, 2 μg/kg, 2.5 μg/kg, 3 μg/kg, 10 μg/kg, 50 μg/kg, 100 μg/kg, 1 mg/kg, 2 mg/kg, 5 mg/kg, 10 mg/kg, 50 mg/kg, or 100 mg/kg, or a range between and including any two of the foregoing values such as about 0.1 μg/kg to 3 μg/kg, about 3 μg/kg to 30 μg/kg, or about 30 μg/kg to about 300 μg/kg, or to about 3 mg/kg, depending on the factors mentioned above. For repeated administrations over several days or longer, depending on the condition, the treatment is sustained until a desired suppression or amelioration of symptoms occurs or until sufficient therapeutic levels are achieved to alleviate a cancer, or one or more symptoms thereof. An exemplary dosing regimen comprises administering an initial dose of about 2 mg/kg, followed by a weekly or biweekly maintenance dose of about 1 mg/kg of the checkpoint inhibitor and/or about 3.75×10⁶ IU/kg of the immune cytokine. However, other dosage regimens may be useful, depending on the pattern of pharmacokinetic decay that the practitioner (e.g., a medical doctor) wishes to achieve. For example, dosing from one to four times a week is contemplated. In some embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy may be monitored by conventional techniques and assays and/or by monitoring the progress of the disease or cancer as described herein. The dosing regimen (including the therapeutic used) may vary over time until a dosage is reached that achieves the desired result. Typically the clinician will administer an immune checkpoint inhibitor, such as an antibody, e.g, an antibody against PD1, PDL1, or CTLA-4.

Administration of an immune checkpoint inhibitor and/or an immune adjuvant can be continuous or intermittent, depending, for example, upon the recipient's physiological condition, whether the purpose of the administration is therapeutic or prophylactic, and other factors known to skilled practitioners. The administration of an immune checkpoint inhibitor (e.g., a PD1 inhibitor or a CTLA4 inhibitor) and/or an immune adjuvant (e.g., IL-2) may be essentially continuous over a preselected period of time or may be in a series of spaced dose, e.g., either before, during, or after developing cancer.

The examples herein are provided to illustrate advantages of the present technology and to further assist a person of ordinary skill in the art with preparing or using the nanoparticles compositions of the present technology. To the extent that the compositions include ionizable components, salts such as pharmaceutically acceptable salts of such components may also be used. The examples herein are also presented in order to more fully illustrate the preferred aspects of the present technology. The examples should in no way be construed as limiting the scope of the present technology, as defined by the appended claims. The examples can include or incorporate any of the variations or aspects of the present technology described above. The variations or aspects described above may also further each include or incorporate the variations of any or all other variations or aspects of the present technology.

### EXAMPLES

Materials. Poly-L-lysine hydrobromide (PLL) with a molecular weight of 30 to 70 kDa, ammonium hydroxide (NH4OH) solution and sucrose were purchased from Sigma-Aldrich. Ferric chloride hexahydrate (FeCl3·6H2O) and ferrous sulfate heptahydrate (FeSO4·7H2O) were purchased from Fisher Scientific. Citric acid was obtained from Acros Organics. CpG oligodeoxynucleotides 1826 (CpG 1826) was purchased from Integrated DNA Technologies. Fluorescein isothiocyanate (FITC) was purchased from Chemodex Ltd. 3-(4,5-dimethylthiazol-2-yl)-2,5-diphenyltetrazolium bromide (MTT) was purchased from VWR International, LLC. Cyanine5-NHS easter (Cy5-NHS) was purchased from Lumiprobe. Ovalbumin (Ova) and fluorescein isothiocyanate labeled ovalbumin (FITC-Ova) were purchased from ThermoFisher. Macrophage colony-stimulating factor (M-CSF), granulocyte-macrophage colony-stimulating factor (GM-CSF) and Interleukin 4 (IL-4) were purchased from Biolegend. α-CTLA-4 (IgG2c, clone 9D9) was produced and purified by Neoclone.

Statistical Analysis. Prism 8 (GraphPad Software) and R (v 4.0.5) were used for all statistical analyses. Unpaired t-test was used for two-group comparisons. One-way ANOVA was used for the analysis of stability of PIC, cytotoxicity, clonogenic assay, cellular uptake, gene expression (in vitro and in vivo), flow cytometry studies and toxicity studies. For B78 tumor re-challenge, time-weighted average of tumor volume was calculated for each mouse, then compared by using Kruskal-Wallis test and following multiple comparison Wilcoxon rank sum test with Benjamini-Hochberg adjustment for p-values. For B78 melanoma tumor growth, MyC-CaP prostate tumor growth, TC11 breast tumor growth, components study and B78 melanoma two-tumor growth, a linear mixed effects model after log transformation of tumor volume data was fitted in which treatment group, day, and two-way interactions were considered as fixed effects. A complete case analysis was used, which discards only the missing measurements of deceased mice, to handle the missing data. A log-rank test was conducted to compare the survival curves, followed by multiple comparison with Benjamini-Hochberg adjustment for p-value. All data presented are reported as mean±SD unless otherwise noted. For all graphs, *, P<0.05; **, P<0.01; ***, P<0.001; and ****, P<0.0001.

### Example 1—Preparation and Physical Characterization of Multifunctional Nanoparticles (PIC NP)

FIG. 1A depicts schematically how an illustrative embodiment of nanoparticles of the present technology were synthesized.

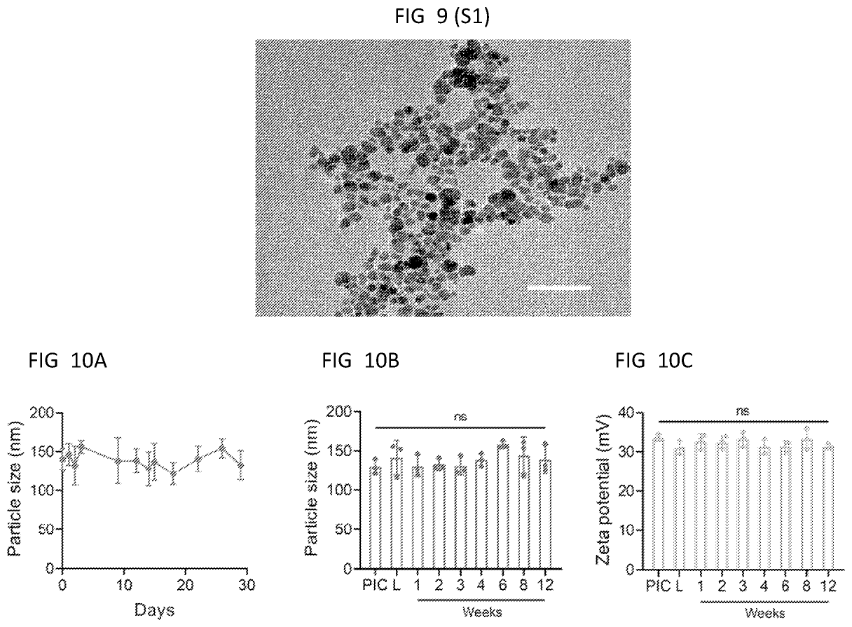

Synthesis of iron oxide nanoparticle (ION). The ION was synthesized according to the previously reported hydrothermal method with minor modification. [1] Briefly, FeCl₃·6H₂O (3.30 g, 12.2 mmol) and FeSO₄·7H₂O (2.37 g, 8.5 mmol) were dissolved in 20 mL deionized water. The iron salt solution was stirred under nitrogen atmosphere for 30 min. After the iron salt solution was heated to 80° C., a solution of NH₄OH (11.25 mL, 28%-30%) was added dropwise. The combined solution was heated to 90° C. before adding a citric acid solution (4.00 mL, 475 mg/mL). The resulting mixture was stirred at 90° C. for 60 min. Dialysis of the mixture against deionized water using dialysis tubing with MWCO: 8000 Da provided the ION had an Fe content of 64.1% as measured by inductively coupled plasma-optical emission spectrometry (ICP-OES). FIG. 9 shows a TEM image of ION.

Preparation of PLL/CpG/iron oxide (PIC NP). ION was mixed with PLL in deionized water at different weight ratios through vortexing (for 20 seconds). The mixture was incubated at room temperature for 20 min. Then, CpG was added and the resulting solution was vortexed for 20 s and incubated at room temperature for another 20 min to yield PIC. PIC was stored at 4° C. in deionized water or in 1 mM PBS, and the particle size was monitored for 30 days. In addition, PIC was lyophilized in the presence of 1% sucrose (wt/wt), and the dry powder was stored at −20° C. for 12 weeks. The particle size and zeta potential were monitored during the storage. Gel electrophoresis was used to confirm the complexation of CpG in the PIC nanoparticle. The weight ratios of PLL/CpG and ION/PLL/CpG were 3.5/1 and 4.8/3.5/1, respectively. The amount of CpG for each sample was 0.75 μg. Electrophoresis was performed using 4% agarose gel and TBE (Tris-Borate-EDTA) buffer with a voltage of 100 V for 15 min.

FITC-labeled and Cy5-labeled PIC. FITC-labeled PIC was prepared through the complexation of FITC-conjugated PLL with ION and CpG, while Cy5-labeled PIC was prepared through the complexation of Cy5-conjugated PLL with ION and CpG. To synthesize FITC-labeled PLL, a FITC solution (3 mL, 0.1 mg/mL in DMSO) was added in PLL aqueous solution (20 mL, with 10 mg PLL), and the mixture was stirred at room temperature in the dark for 24 hours. To synthesize Cy5-labeled PLL, a Cy5-NHS solution (20 μL, 5 mg/mL in DMSO) was added into a PLL aqueous solution (10 mL, with 10 mg PLL and 60 mM NaHCO₃), and the mixture was stirred at room temperature in the dark for 24 hours. The FITC-conjugated PLL and Cy5-conjugated PLL were obtained by dialysis against deionized water using a dialysis tubing (MWCO: 3500) followed by lyophilization. FITC-labeled PIC or Cy5-labeled PIC were prepared using the dye-labeled PLL through the same method mentioned above.

Nanoparticle Characterization Techniques. The hydrodynamic diameters and zeta potentials of the nanoparticles (ION and PIC NP) were characterized by a dynamic light scattering (DLS) spectrometer (Malvern Zetasizer Nano ZS) at a 90° detection angle with a sample concentration at 0.1 mg/mL and pH of 7.4 at 25° C. The morphologies of ION and PIC NP were characterized by transmission electron microscopy (TEM, Philips CM200 Ultra Twin). The Fe content in ION was measured by inductively coupled plasma-optical emission spectrometry (ICP-OES, Agilent 5110).

### Results

To prepare PIC via a scalable and straightforward complexation process, the negatively charged ION was first mixed with positively charged PLL, which was subsequently complexed with negatively charged CpG. Nanoparticles with different particle sizes and zeta potentials were obtained by varying the weight ratio between ION, PLL and CpG (Table 1). Because positively charged nanoparticles with high zeta potentials can interact strongly with negatively charged TAAs, we chose a PIC with a weight ratio of ION/PLL/CpG as 4.8/3.5/1 for further study, as this particle exhibited maximal zeta potentials for particle sizes deemed suitable for this application (FIGS. 1D-1D). Agarose gel electrophoresis assay confirmed highly efficient loading of CpG on the PIC (FIG. 1G). The PIC nanoparticle was stable at 4° C. at least for 30 days (FIG. 1H and FIG. 10A). And the lyophilized PIC showed negligible changes in particle size and zeta potential during storage at −20° C. for at least 12 weeks (FIGS. 10B-10C).

### Table 1. Particle Size and Zeta Potential of ION/PLL/CpG Nanoparticles

ION/PLL/CpG (w/w/w)* Particle Size (nm) Zeta potential (mV)
   9.6/2/3 141.5 nm (PDI: 0.231)−30.5, −30.5, −29.8
   9.6/4/3 precipitation
   9.6/7/3 121.8 nm (PDI: 0.127) 24.6, 23.3, 24.4
   4.8/3.5/1 113.2 nm (PDI: 0.302) 32.8, 34, 34.3 * w/w/w indicates the weight ratio between different components

### Example 2-Biological Activity of PIC NP

Cell culture. B78 (B78-D14, GD2+) melanoma originated from B16 melanoma and was obtained from Ralph Reisfeld (Scripps Research Institute) in 2002. [2] B16 melanoma cells were obtained from Memorial Sloan Kettering Cancer Center. MyC-CaP and RAW264.7 cells were purchased from ATCC (MyC-CaP: CRL-3255; RAW264.7: TIB-71). Panc02 pancreatic cancer cells were obtained from the National Cancer Institute. B16 cells were transduced to express SIINFEKL via lentiviral transduction pLV [Exp]-Hygro-CBh>SIINFEKL (VectorBuilder; VB210327-1014dyd), which is a lentiviral plasmid that we designed using VectorBuilder's platform. Positively transduced cells were referred to as B16-SIINFEKL (a kind gift from Dr. Amy Erbe), and were selected for using hygromycin (50 ug/ml). Stably transduced cells were single-cell cloned. Clones were selected for downstream use following IFNγ (100 U/mL; cat #505702, Biolegend) stimulation, and screened for MHC-I presentation of SIINFEKL via flow cytometry on an Attune NxT Flow Cytometer (Thermofisher) using anti-mouse H-2Kb bound SIINFEKL-APC (clone 25-D1.16, cat #141605, Biolegend). TC11 cells were generated from an ER+ mammary tumor that developed in an NRL-PRL female. [3] B78, B16, B16-SIINFEKL, MyC-CaP, Panc02 and RAW264.7 cells were cultured in Roswell Park Memorial Institute (RPMI)-1640 medium. TC11 cells were cultured in Dulbecco's Modified Eagle Medium (DMEM). Primary macrophages were derived from bone marrow (BMDM) and cultured in Modified Eagle Medium (MEM) supplemented with 12 ng/ml M-CSF. Dendritic cells (DCs) were enriched from the splenocytes that were collected from Flt3L injected C57BL/6 mice using an Easy Sep™ Mouse Pan-DC Enrichment Kit (STEMCELL), and cultured in RPMI-1640 medium supplemented with 25 ng/mL GM-CSF and 20 ng/ml IL-4. RPMI-1640 medium, MEM and DMEM were supplemented with 10% (vol/vol) fetal bovine serum (FBS), 100 U/mL penicillin and 100 μg/mL streptomycin. Cell authentication was performed per ATCC guidelines using morphology, growth curves and Mycoplasma testing within 6 months of use and routinely thereafter. All the cells were cultured at 37° C. in a humidified atmosphere containing 5% CO₂.

Cell lysate absorption of PIC NP in vitro. B78 melanoma cell lysates were prepared by ultrasonication. The concentration of proteins in the cell lysate was determined by BCA assay (ThermoFisher). To determine cell lysate absorption, PIC (0.1 mL, 0.7 mg/mL) was added to the B78 cell lysates (0.4 mL). B78 cell lysates (0.4 mL) dispersed in deionized water (0.1 mL) was used as a control. The mixtures were incubated at 37° C. for 4 hours followed by centrifugation at 12000×g for 20 min. The concentrations of proteins in the supernatants were determined by BCA assay.

Cytotoxicity assay. B78 cells and RAW264.7 cells were seeded and cultured in 96-well plates with 8,000 cells per well 24 hours before treatments. Cells were treated with PIC at different concentrations (i.e., 0, 1.1, 2.2, 4.4, 8.8, 17.5 and 35 μg/mL). After incubation for 48 hours, cells were washed with PBS three times and subsequently treated with fresh medium containing 0.5 mg/mL MTT. After incubation for another 4 hours, the medium was aspirated and 150 μL DMSO was added to each well to dissolve the formazan product. The absorbance at 560 nm was then measured using a GloMax-Multi Microplate Multimode Reader (Promega). Cell viability was calculated as a percentage of the absorbance relative to that of untreated cells.

Confocal laser scanning microscopy. Confocal laser scanning microscopy (CLSM) was carried out. B78 cells and RAW264.7 cells were seeded on coverslips in 6-well plates and incubated for 24 hours. Cells were treated with FITC-labeled PIC at 3.8 μg/mL. After 2 hours of incubation, cells were washed with PBS and fixed with 4% paraformaldehyde. Thereafter, cells were stained by 4′-6-diamidino-2-phenylindole (DAPI, 1 mg/mL, 1 μL/well) and washed with PBS. The coverslips were carefully taken out from the wells, placed on slides and enclosed with anti-fade mounting medium. The samples were imaged with a Nikon A1R-Si high speed spectral laser scanning confocal inverted microscope (Nikon, Melville).

In vitro cellular uptake by flow cytometry. B78 cells and RAW264.7 cells were seeded and cultured in 96-well plates with 10,000 cells per well 24 h before treatments. Cells were treated with FITC-labeled PIC NP at different concentrations (i.e., 1.9, 3.8 and 7.5 μg/mL). Two hours later, the cells were collected with trypsinization and centrifugation. The cellular uptake was measured by flow cytometry (Attune NxT flow cytometer system, ThermoFisher, USA) measuring FITC-positive cells.

In vitro immunofluorescence of damaged DNA. B78 cells were seeded on coverslips in 12-well plates with 1×10⁵ cells per well and incubated at 37° C. for 24 hours. Cells were treated with PIC at 4.67 μg/mL. Four hours later, radiation (12 Gy, RS225 Cell Irradiator (Xstrahl)) was delivered to the cells. After another 1 hour of incubation, the cells were washed with PBS and fixed with 4% paraformaldehyde solution. After the cells were permeabilized with 0.1% Triton X-100, non-specific binding was blocked with SuperBlock (TBS T20, ThermoFisher). Then primary antibody (Phospho-Histone H2A.X (Ser139) (20E3) Rabbit mAb, #9718, Cell Signaling Technology, 1:400) was applied and incubated overnight at 4° C. After washing, a fluorescently labelled secondary antibody (Catalog #A-11008, ThermoFisher) was applied and incubated for 1 hour at room temperature, the cell nuclei were stained with DAPI. Then the coverslips were washed with PBS and DI water, and then carefully taken out from the wells, placed on slides and enclosed with anti-fade mounting medium. The samples were imaged with a Nikon A1R-Si high speed spectral laser scanning confocal inverted microscope (Nikon, Melville).

cGAS-STING activation. B78 cells were seeded in 6-cm cell culture plates with 1×10⁶ (for the collection on day 1), 0.5×10⁶ (for the collection on day 4) or 0.25×10⁶ (for the collection on day 7) cells per well. After incubation overnight, the following treatments were performed on the cells: (1) un-treated; (2) radiating the cells at a dose of 12 Gy and changing to fresh media 1 hour after the radiation; (3) adding PIC at a concentration of 4.67 μg/mL; (4) adding PIC at a concentration of 4.67 μg/mL, radiating the cells at a dose of 12 Gy 4 hours later, and changing to fresh media after another 1 hour; and (5) radiating the cells at a dose of 12 Gy and changing to fresh media containing 4.67 μg/mL PIC at 1 hour later. At day 1, 4 and 7, the cells were washed with cold PBS three times followed by direct addition of Trizol (1 mL). RNA was extracted using RNeasy Mini Kit (QIAGEN, Cat #74106) according to the manufacturer's instructions. cDNA was synthesized using QuantiTect Reverse Transcription Kit (QIAGEN, Cat #205314) according to the manufacturer's standard protocol. Quantitative polymerase chain reaction (RT-qPCR) was performed using Taqman Fast Advanced Master Mix and predesigned Taqman gene expression assays for Ifnβ1 and Pd-l1. Thermal cycling conditions (QuantStudio 6, Applied Biosystems) included the UNG incubation stage at 50° C. for 2 min, followed by AmpliTaq™ Fast DNA polymerase activation stage at 95° C. for 2 min followed by 40 cycles of each PCR step (denaturation) 95° C. for Is and (annealing/extension) 60° C. for 20s. For data analyses, Ct values were transferred to an Excel file and fold change was determined using the ΔΔCt method. HPRT was used as the endogenous control.

Polarization of macrophages. BMDMs were seeded in 6-cm cell culture plates with 0.5×10⁶ cells per well. After incubation overnight, fresh media containing 6 ng/ml M-CSF was changed, and the following treatments were performed on these cells: (1) un-treated; (2) radiating the cells at a dose of 12 Gy and changing fresh media 1 hour after the radiation; (3) adding PIC at a concentration of 4.67 μg/mL; (4) radiating the cells at a dose of 12 Gy and changing fresh media containing 4.67 μg/mL PIC at 1 hour later. When the culture media was changed, 6 ng/ml M-CSF was supplemented. At day 1 and 4, the cells were collected and stained with surface antibodies: anti-F4/80 PE-Dazzle594, anti-CD206 BV421, anti-CD11b BV711, anti-CD80 APC, Live/Dead Ghost Red 780. After the cells were fixed and permeabilized, anti-CD163 PE-Cy7 was added to the cells for intracellular staining. The UltraComp Beads eBeads (Invitrogen) were used for compensation. All samples were incubated with CD16/CD32 (Fc block) for 5 minutes at room temperature before staining. Flow cytometry was performed on an Attune Cytometer (ThermoFisher).

Clonogenic assay in vitro. Clonogenic assays were used to evaluate the potential effect of PIC NP on the intrinsic radiosensitivity of tumor cells. The in vitro clonogenic assays were performed as previously described [4]. 1000 B78 cells were plated into the 6-cm cell culture plates. One day later, ION, PIC NP or vehicle control solution was added with a final concentration of 4.7 μg/mL. 4 hours later, radiation treatment was delivered at doses of 0 Gy, 3 Gy, 6 Gy or 9 Gy to the cells. Fresh culture media was changed 1 hour after radiation. 7 days later, when the control plates had sufficient colonies formed, the cell medium was discarded, and plates were rinsed with PBS and the colonies were fixed using 6% glutaraldehyde and 0.5% crystal violet for 30 minutes. Then, the cells were rinsed carefully with tap water and dried at room temperature. [5] The colonies were counted using stereomicroscope and colony counter pen, the log surviving fraction of control and RT treated colonies were calculated and plotted.

### Results

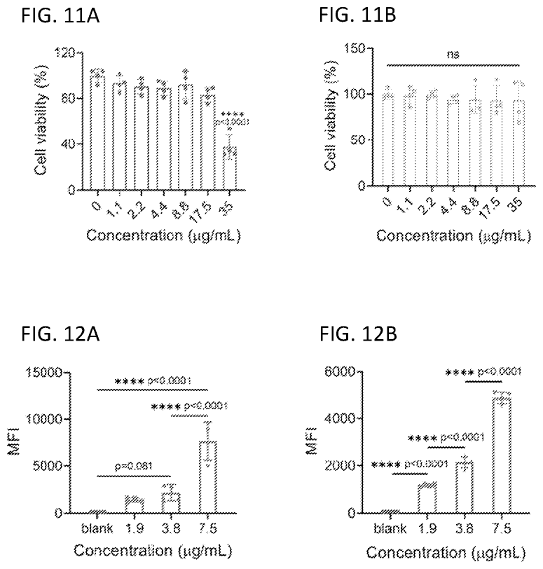

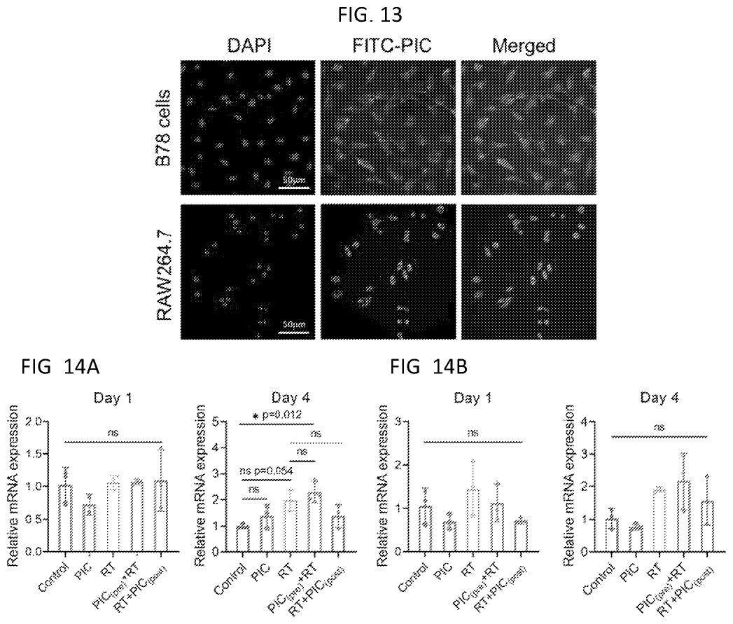

To test the protein absorption ability of PIC, cell lysates were prepared from B78 murine melanoma cells. As shown in FIG. 1I, incubation with PIC (0.14 mg/mL) caused a significant decrease of protein concentration in the tumor cell lysates, indicating the strong antigen capture ability of PIC. PIC did not show any direct cytotoxicity to either murine macrophage cell line RAW264.7 or B78 melanoma cells when the concentration was lower than 17.5 μg/mL (FIGS. 11A-11B). Upon analysis by both flow cytometry and confocal laser scanning microscopy (CLSM), PIC showed a dose-dependent cellular uptake in RAW264.7 macrophages, and cellular uptake was also observed at higher dose in B78 melanoma cells although this was only statistically significant at 7.5 μg/mL (FIGS. 12A-12B and 13). Prior studies demonstrate that iron oxide nanoparticles can sensitize tumor cells to radiation. Using the in vitro clonogenic assay, a standard approach to quantifying radiosensitivity, we confirmed that PIC significantly increased the sensitivity of B78 melanoma cells to RT (FIG. 1J). Immunofluorescence microscopy quantifying γH2AX foci confirmed that this effect in B78 melanoma cells correlated with a role of PIC enhancing the DNA damage resulting from RT, although the PIC alone did not directly induce DNA damage, as expected (FIGS. 1K-1L).

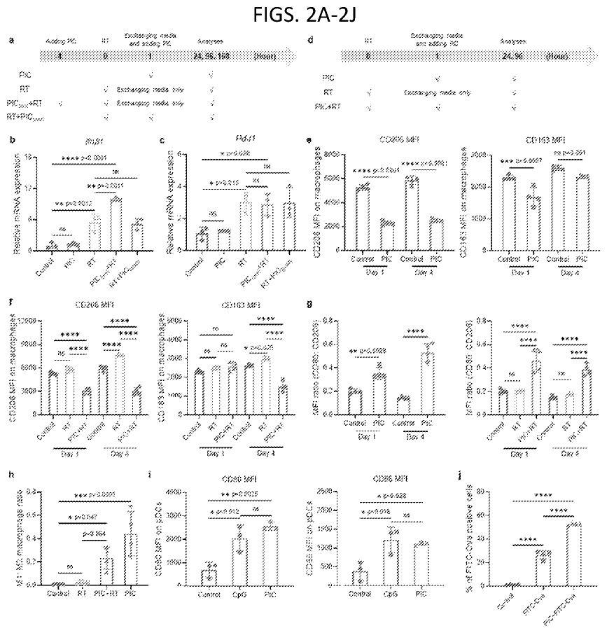

Activation of an IFN-I response via the cGAS/STING pathway in tumor cells has been shown to be critical for the effects of RT in stimulating an in situ vaccine effect and potentiating response to ICBs. We previously observed that the activation of IFN-I via cGAS/STING pathway in B78 cells peaked at day 7 post-RT. In B78 melanoma, PIC alone did not significantly influence the expression of Ifnβ1, a marker of cGAS/STING activation of an IFN-I response (FIGS. 2A-2B), and elicited negligible effects on the Ifnβ1 expression in the radiated B78 cells when it was added after RT. However, pre-treatment with PIC before RT significantly increased the expression of Ifnβ1 in B78 cells at day 7 post-treatment compared to RT alone (FIGS. 2B and 14A). Although RT is also noted to increase expression of Pd-l1 in tumor cells, PIC did not influence Pd-l1 expression in B78 cells after RT at tested timepoints (FIGS. 2C and 14B).

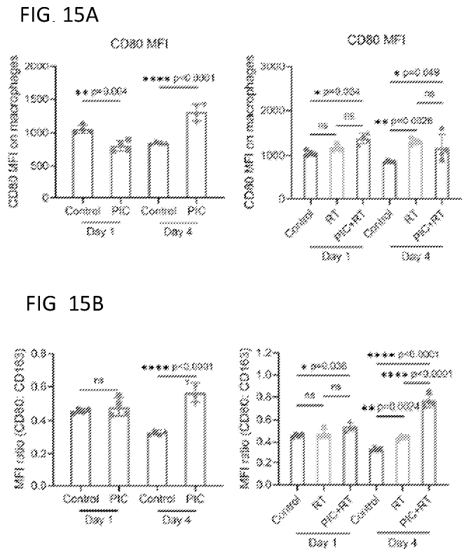

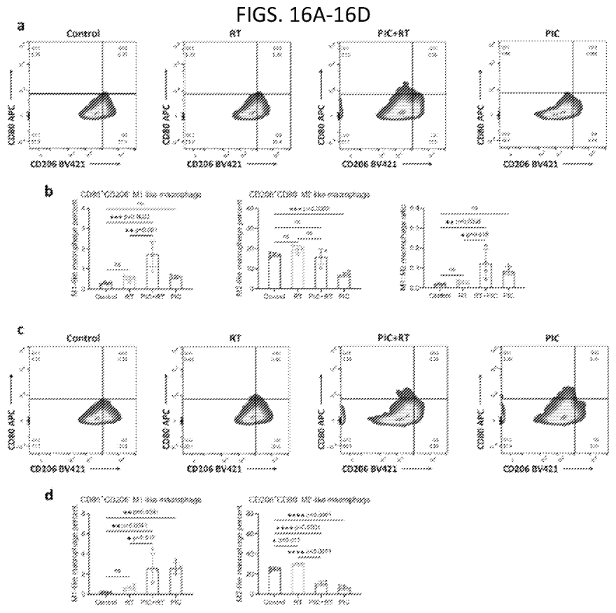

We evaluated the effect of PIC on the polarization of bone marrow derived macrophages (BMDMs) (FIG. 2D). RT significantly upregulated the expression of CD206 and CD163, two markers of M2 macrophages, in CD11b⁺F4/80⁺ BMDMs at day 4 after treatment, while PIC significantly downregulated expression of these markers and increased the expression ratios of CD80:CD206 and CD80:CD163 in both non-radiated and radiated CD11b⁺F4/80⁺ BMDMs (FIGS. 2E-2G and 15A-15B). Consequently, PIC treatment reduced the percentage of CD206⁺CD80⁻ M2-like macrophages and enhanced the percentage of CD80⁺CD206⁻ M1-like macrophages among CD11b⁺F4/80⁺ BMDMs at day 4 after RT, confirming the PIC achieved its intended effects on macrophage polarization (FIGS. 2H and 16A-16D).

TLR9 activation and antigen uptake by DCs. DCs were seeded in 12-well plates with 2×10⁵ cells/well and incubated for 24 hours. The following treatments were performed on these cells: (1) un-treated; (2) adding CpG at a concentration of 0.5 μg/mL; (3) adding PIC at a concentration of 4.67 μg/mL. 24 hours later, the supernatants were collected for the quantitative analysis of IFN-β using a mouse IFN-β Elisa kit (Biolegend, Cat #439407), and the cells were collected and stained with surface antibodies: anti-CD11c PerCP-Cy5.5, anti-CD80 PE, anti-CD86 BV605, anti-CD317 Alexa 700, Live/Dead Ghost Red 780. To study the cellular uptake of FITC-Ova, the following treatments were performed on the DCs: (1) un-treated; (2) adding FITC-Ova at a concentration of 1.67 μg/mL. (3) adding the mixture of FITC-Ova and PIC (1.67 μg/mL FITC-Ova and 4.67 μg/mL PIC). The FITC-Ova and PIC were mixed 20 minutes before adding to the cells). 24 hours later, the cells were collected and stained with anti-CD11c PerCP-Cy5.5 and Live/Dead Ghost Red 780. The UltraComp Beads eBeads (Invitrogen) were used for compensation for flow cytometry. All samples were incubated with CD16/CD32 (Fc block) for 5 minutes at room temperature before staining. Flow cytometry was performed on an Attune Cytometer (ThermoFisher).

Analysis of T cells. C57BL/6 female mice (7-8 weeks) were randomized into three groups and subcutaneously injected with 100 μL of (1) the mixture of Ova and PIC (1.4 mg/mL of PIC and 0.5 mg/mL of Ova. PIC and Ova were mixed 20 minutes before injection); (2) Ova solution (0.5 mg/mL of Ova); (3) the mixture of Ova with CpG and ION (0.5 mg/mL of Ova, 0.15 mg/mL of CpG and 0.72 mg/mL of ION. Ova, CpG and ION were mixed 20 minutes before injection). At day 13 after the injection, B16-SIINFEKL cells were seeded in 6-well plates with 0.5×10⁶ cells per well. At day 14, the mice were euthanized and the spleens were collected aseptically, dissociated into a single cell suspension, incubated in RBC lysis buffer for 10 mins and then PBS was added to neutralize the lysis buffer. 0.5×10⁶ splenocytes were added to the B16-SIINFEKL culture and these cells were incubated overnight. The next day, 1 μL/sample of BD Cytofix/Cytoperm Plus kit was added to the cells for 4-6 hours. The cells were collected and stained with surface antibodies: anti-CD4 FITC, anti-CD69 PE-Cy5, anti-CD45 PE-Cy7, anti-CD3 BV605, anti-CD44 BV711, anti-CD8a Alexa 700 and Ghost Red 780. After the cells were fixed and permeabilized, anti-IFN-γ PE-Dazzle 594 was added to the cells for intracellular staining. UltraComp Beads eBeads (Invitrogen) were used for compensation. All samples were incubated with CD16/CD32 (Fc block) for 5 minutes at room temperature before staining. Flow cytometry was performed on an Attune Cytometer (ThermoFisher).

### Results

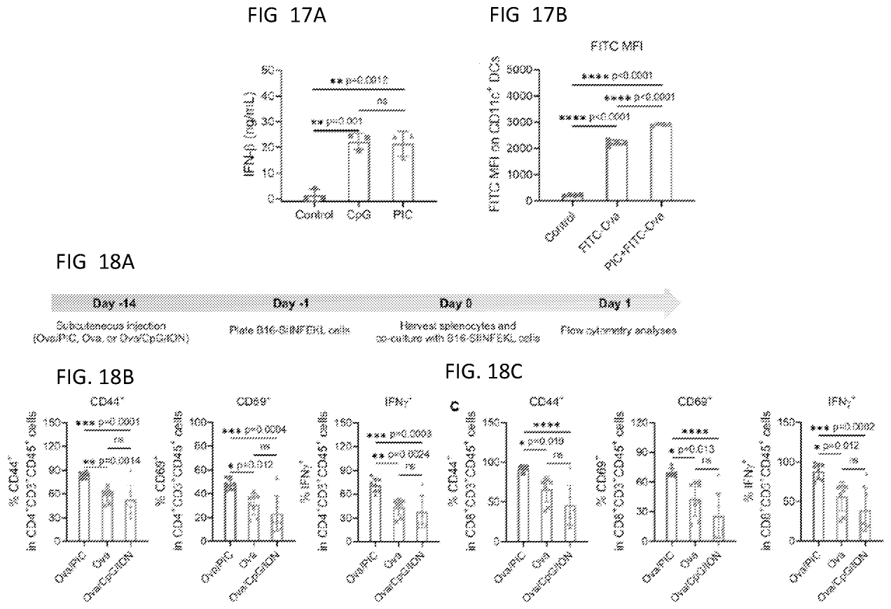

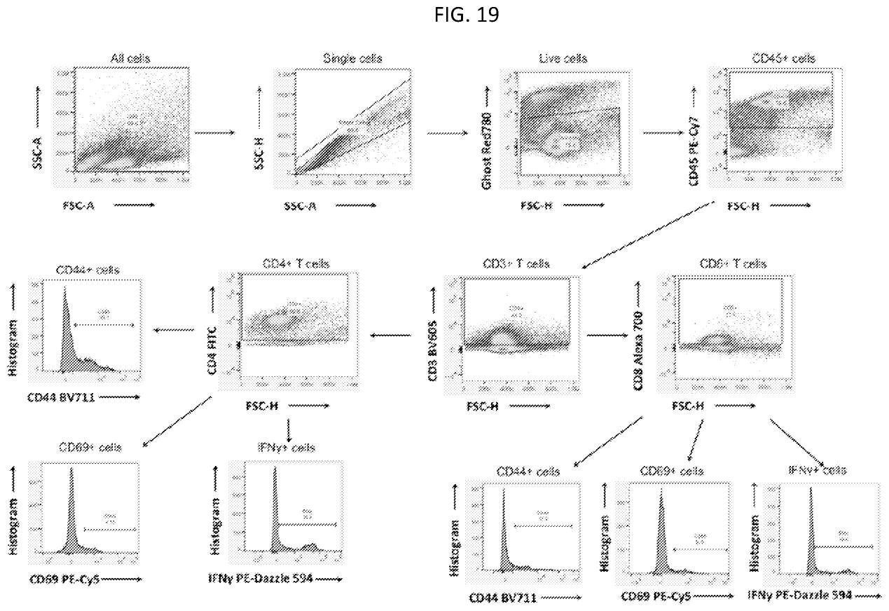

We evaluated the capacity of PIC to activate dendritic cells (DCs) and enhance antigen presentation. Mouse DCs were isolated from the spleen and treated with PIC in vitro. We found PIC treatment markedly increased the expression of CD80 and CD86 on the surface of CD11c⁺CD317⁺ plasmacytoid dendritic cells (pDCs), which express endosomal TLR-9 that is activated by CpG (FIG. 2I). PIC-activated DCs exhibited production of IFN-β, suggesting the potential for PIC to directly influence the tumor immune microenvironment at an injected site (FIG. 17A). To test the ability of PIC to improve the antigen uptake by APCs, we treated DCs with pre-complexed PIC and FITC-labeled ovalbumin (FITC-Ova) and compared the internalization of FITC-Ova in CD11c⁺ cells with those treated with FITC-Ova alone. PIC significantly improved the cellular uptake of FITC-Ova by CD11c″ DCs (FIGS. 2J and 17B). To determine whether the effects of PIC on antigen presenting cells would augment antigen presentation and enhance T cell immunity, we injected healthy mice with pre-complexed Ova/PIC, Ova/CpG/ION, or Ova alone. We then isolated splenocytes from these mice and co-cultured these with B16-SIINFEKL cells (FIG. 18A). Both CD4⁺ and CD8⁺ T cells from splenocytes of mice injected with Ova/PIC showed higher expression of CD69 (early T cell activation marker), CD44 (effector T cell marker), and IFNγ (key mediator of T cell activation) when compared to those from Ova or Ova/CpG/ION injected mice (FIGS. 18B-18C and 19). These results demonstrate that PIC can promote DC activation and antigen cross presentation enabling robust T cell immunity.

### Example 3—the Immune Modulation by PIC In Vivo

### Methods:

Tumor models. All mice (C57BL/6 and FVB/NTac, 7-8 weeks) were purchased from Taconic. All mice were maintained under a tightly controlled temperature (22° C.), humidity (40-50%), light/dark (12/12 h) cycle conditions, with water and food ad libitum. To establish tumor-bearing mice (C57BL/6 male and female mice for B78, and FVB/NTac male mice for MyC-CaP), mice were intradermally engrafted with tumor cells (B78 melanoma model: 2×10⁶ cells on right flank; MyC-CaP prostate tumor model: 1×10⁶ cells on right flank; B78 melanoma two-tumor model: 2×10⁶ cells on right flank, and one week later 2×10⁶ cells engrafted on left flank). For TC11 breast tumor model, 5×10⁴ TC11 cells were injected on the mammary fat pad of female FVB/NTac mice. Once tumor volumes reached approximately 100 mm³, mice were randomized and then treatment was begun. Tumors were measured twice weekly for at least 60 days after starting treatment unless mice died or were euthanized because of large tumor size (according to the animal study protocol, mice were euthanized when the diameter of tumors was approximately 20 mm), tumor necrosis, or evidence of pain or distress. Tumor diameters were measured with a Vernier caliper, and tumor volume was calculated through the equation: tumor volume=longer diameter×shorter diameter²×0.5.

Biodistribution. Cy5-labeled PIC (100 μL, 1.4 mg/mL) was intratumorally injected into the B78 melanoma bearing mice. The whole body of the mice was scanned with an in vivo imaging system (IVIS) at 3, 8, 24, 48, and 72 hours after injection. For each scan, mice were anesthetized with isoflurane (4% induction and 2% maintenance) and placed on the scanner bed in a prone position. The mice were shaved in the tumors and tumor-draining lymph nodes sites before the scan.

For flow cytometry, mice were euthanized and the tumors and tumor-draining lymph nodes were collected at 3 hours after injection. Tumors and tumor-draining lymph nodes were enzymatically dissociated with DNase and collagenase on a Gentle MACS Octodissociator (Miltenyi Biotec) and then filtered through a 70 μm cell strainer and red blood cells were lysed using RBC lysis buffer. Single cell suspensions were stained with surface antibodies: anti-CD11c FITC, anti-F4/80 PE-Dazzle594, anti-CD45 PE-Cy7, anti-MHCII BV510, anti-CD11b BV711 and Live/Dead Ghost Red 780. The UltraComp Beads eBeads (Invitrogen) were used for compensation. All samples were incubated with CD16/CD32 (Fc block) for 5 minutes at room temperature before staining. Flow cytometry was performed on an Attune Cytometer (ThermoFisher).

RT-qPCR gene expression study in vivo. Tumor samples were collected on treatment day 15 from B78 melanoma bearing mice and homogenized using a Bead Mill Homogenizer (Bead Ruptor Elite, Omni International). Total RNA was extracted after sample homogenization using RNeasy Mini Kit (QIAGEN) according to the manufacturer's instructions. cDNA was synthesized using QuantiTect Reverse Transcription Kit (QIAGEN) according to the manufacturer's instructions. Quantitative polymerase chain reaction (RT-qPCR) was performed using Taqman Fast Advanced Master Mix and predesigned Taqman gene expression assays for Ifnb1, Mx1, Arg1, Nos2, Ifnγ, Il6, Tnfα, Il1β, Pd-l1, Il10 and Tgfβ1. Thermal cycling conditions and data analysis were as indicated above and HPRT was used as the endogenous control.

Analysis of immune cells in tumors and tumor-draining lymph nodes. Tumor and tumor-draining lymph node samples were collected on day 15 post-initiation of treatment from B78 melanoma bearing mice for flow cytometry analysis (all the lymph nodes from each group were combined for analysis due to small size). Briefly, samples were enzymatically dissociated with DNase and collagenase on a Gentle MACS Octodissociator (Miltenyi Biotec) and then filtered through a 70 μm cell strainer and red blood cells were lysed using RBC lysis buffer. The single cell suspensions were divided for the innate immune cells staining and adaptive immune cells staining separately. For innate immune cells staining, the single cell suspensions were stained with surface antibodies: anti-CD11c FITC, anti-MHCII PerCP-Cy5.5, anti-F4/80 PE-Dazzle594, anti-CD45 PE-Cy7, anti-CD103 BV421, anti-CD206 BV605, anti-CD11b BV711, anti-CD80 APC, anti-CD317 Alexa 700, Live/Dead Ghost Red 780. For adaptive immune cells staining, the single cell suspensions were stained with surface antibodies: anti-CD4 FITC, anti-CD69 PE-Cy5, anti-CD45 PE-Cy7, anti-PD-1 BV421, anti-CD62L BV510, anti-CD3 BV605, anti-CD44 BV711, anti-CD25 APC, anti-CD8a Alexa 700, Live/Dead Ghost Red 780. After the cells were fixed and permeabilized, anti-FOXP3 PE was added to the cells for intracellular staining. The UltraComp Beads eBeads (Invitrogen) were used for compensation. All samples were incubated with CD16/CD32 (Fc block) for 5 minutes at room temperature before staining. Flow cytometry was performed on an Attune Cytometer (ThermoFisher) and compensation matrix and data were analyzed using FlowJo software following published flow cytometry guidelines. [6]

In vivo treatments. PIC (100 μL, 1.4 mg/mL) was intratumorally injected on days 0, 3, 6, and 9. The injection time points were selected to fulfill the various intended functions of PIC, which may occur at different time points relative to RT. External beam radiation therapy (EBRT) was delivered to the targeted tumors with a dose of 12 Gy on treatment day 1 using an XRad 320 cabinet irradiator ((Precision X-Ray, Inc) with custom lead shielding of tissues outside of the targeted tumor site. At day 3, 6, 9, anti-CTLA-4 (IgG2c, clone 9D9, 100 μL, 1 mg/mL) was intraperitoneally injected into the mice. Tumors were measured as described above.

Evaluation of immune memory (in vivo). At day 91 after the first injection (see in vivo treatment under General Methods above), tumor-free mice in the PIC NP+RT+anti-CTLA-4 group were re-challenged by engraftment of 2×10⁶ B78 melanoma cells on the left flank. A group of age-matched naïve mice were also engrafted with 2×10⁶ B78 melanoma cells for tumor growth as control. Tumor growth at these sites was monitored for another 50 days. After 50 days, the mice were euthanized and their spleens were removed.

Evaluation of immune memory (in vitro). B16 melanoma cells were plated in 12 well plates (50,000 cells per well) and irradiated with sham or 8 Gy radiation in a single fraction. Five days following irradiation, spleens from naïve and re-challenged disease-free mice were collected aseptically, dissociated into a single cell suspension, incubated in RBC lysis buffer for 10 mins and then an equal amount of PBS was added to neutralize the lysis buffer. Splenocytes were then washed with PBS. 1×10⁶ splenocytes were added to each B16 culture and these cells were incubated overnight. The next day, 1 μL/sample of BD Cytofix/Cytoperm Plus kit was added to the cells for 4-6 hours. The cells were then labeled with antibodies (CD45 PE-Cy7, CD3 FITC, CD4 BV510, CD8 PerCp Cy5.5, CD69 BV421 and Ghost Red 780) and flow cytometry was performed using an Attune cytometer (ThermoFisher) and UltraComp Beads (Invitrogen) for compensation. [6]

Toxicity assays. At day 7, 14, and 21 after the first injection, mice were euthanized and the blood and major organs (liver, kidney, spleen, intestine and femur) were collected from B78 melanoma-bearing mice. Blood metabolic profile analysis was performed using the VetScan Preventive Care Profile Plus rotors (Abaxis) in a VetScan VS2 blood chemistry analyzer (Abaxis). Complete blood count was analyzed by a VetScan HM5 hematology analyzer (Abaxis). Moreover, to evaluate the systemic or local toxicity, the major organs (liver, spleen, kidney, intestine and femur) were sectioned and stained with hematoxylin and eosin (H&E) and observed under an optical microscope.

Components study. On day 0, 3, 6 and 9, 50 μL of each component (PLL: 1.05 mg/mL; ION: 1.42 mg/mL; CpG: 0.3 mg/mL) was injected into different sites of a single B78 tumor separately. EBRT was delivered to the tumor with a dose of 12 Gy on treatment day 1 using an XRad 320 cabinet irradiator with custom lead shielding of tissues outside of the targeted tumor site. At day 3, 6, 9, anti-CTLA-4 (IgG2c, clone 9D9, 100 μL, 1 mg/mL) was intraperitoneally injected into the mice. Tumors were measured as described above.

### Results

### The Immune Modulation by PIC In Vivo and its Effects on the Tumor Infiltrating Immune Cells.

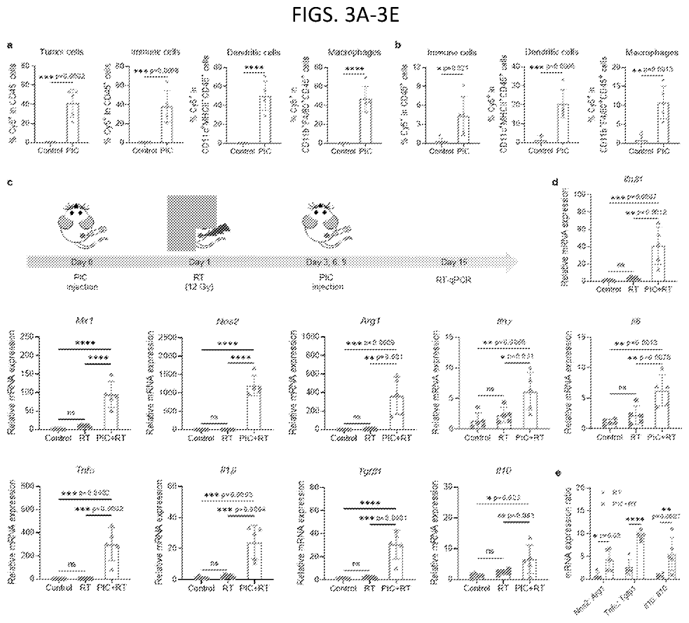

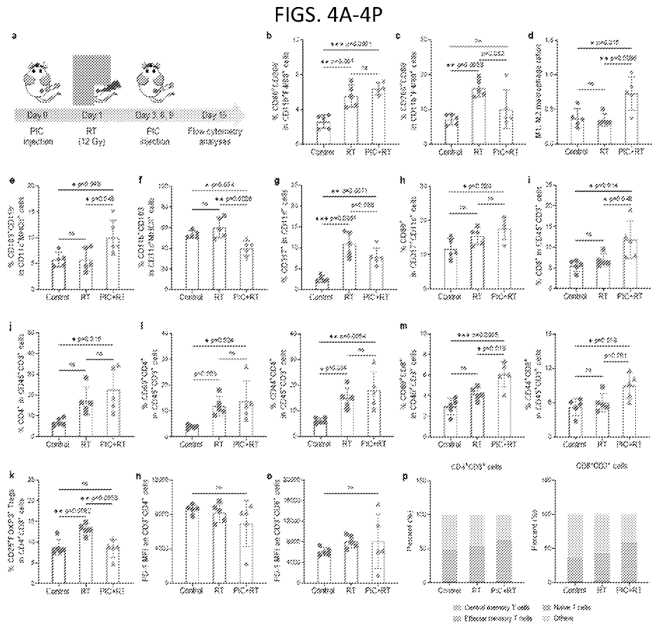

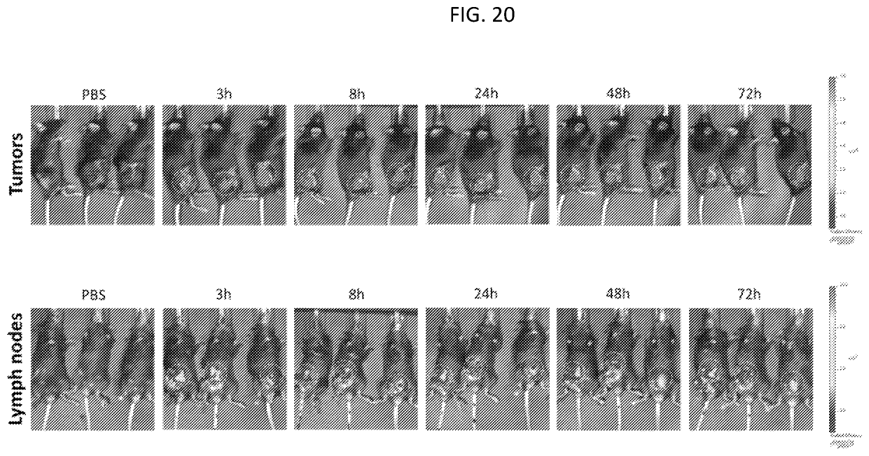

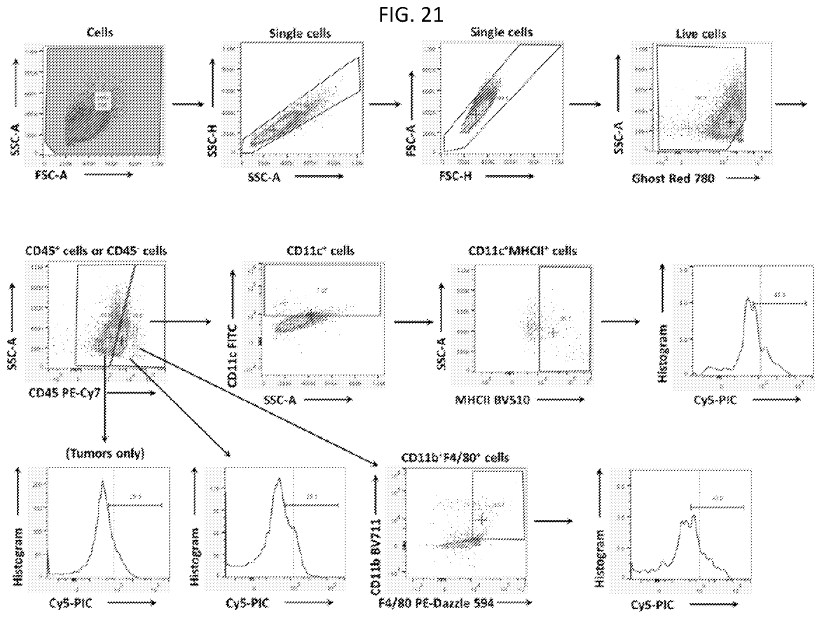

Before studying the ability of PIC to immunomodulate radiated tumor microenvironments, we evaluated its retention in tumor after intratumoral injection and its cellular uptake by tumor cells, macrophages and DCs in vivo. Three days after the Cy5-labeled PIC (Cy5-PIC) was intratumorally injected into B78 melanoma flank tumors, we observed strong fluorescence signal of Cy5 in the tumor sites (FIG. 20). Using flow cytometry on tumors disaggregated 3 hours after intratumoral injection of Cy5-PIC, we found Cy5-PIC was endocytosed and retained by tumor cells, DCs (CD11c⁺MHCII⁺CD45⁺), and macrophages (CD11b⁺F4/80⁺CD45⁺) (FIGS. 3A and 21). Notably, we also observed Cy5-PIC in DCs (CD11c⁺MHCII⁺CD45⁺) and macrophages (CD11b⁺F4/80⁺CD45⁺) upon disaggregation of tumor-draining lymph nodes (TDLNs) after intratumoral injection (FIG. 3B).

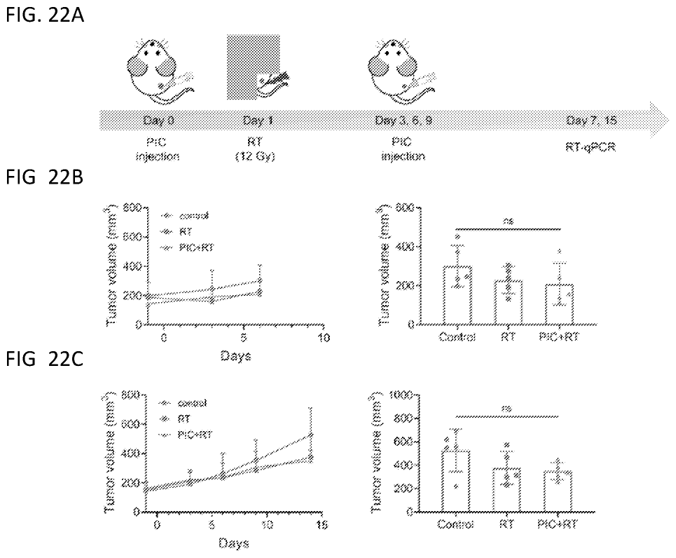

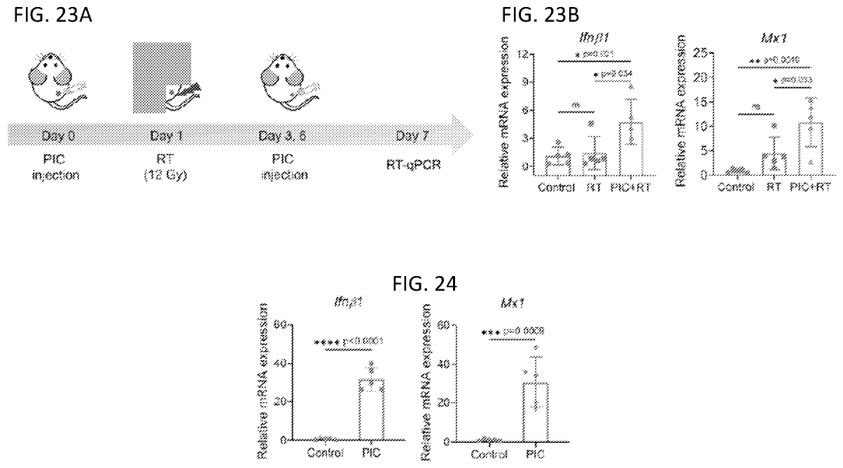

By analyzing bulk tumor mRNA using RT-qPCR, we evaluated the immunomodulatory effect of RT and PIC+RT on B78 tumors (FIGS. 3C and 22A-22C). Consistent with our in vitro data (FIG. 2B), at day 7 after RT we observed increased expression of Ifnβ1 and Mx1, markers of cGAS/STING activation, in tumors treated with PIC+RT compared to those treated with RT alone (FIGS. 23A-23B). This effect was amplified further at day 15 post-treatment (FIG. 3D). From the data shown in FIG. 24, PIC injection alone also enhanced the expression of Ifnβ1 and Mx1 in B78 tumors, which may result from the activation of TLR-9 in endocytic vesicles of antigen presenting cells by the CpG in PIC. Nitric oxide synthase 2 (Nos2) and arginase 1 (Arg1) play important roles in regulating the functions of macrophages and serve as transcriptional markers for M1 and M2 polarization, respectively. As shown in FIG. 3D, both Arg1 and Nos2 mRNA levels increased in the RT+PIC group compared to the RT alone treatment group at day 15, but the magnitude of Nos2 mRNA elevation was far greater than that of Arg1 mRNA (76-fold elevation for Arg1 vs 490-fold elevation for Nos2), resulting in a higher ratio of Nos2:Arg1 in the PIC+RT group compared to the RT group (FIG. 3E). IFN-γ is produced by immune cells and promotes the activity of T helper type 1 cells and CD8⁺ cytotoxic T lymphocytes. We found that Ifnγ expression increased in B78 tumors at day 15 after PIC+RT treatment compared to RT alone (FIG. 3D). PIC also increased the expression of both pro-inflammatory genes (Tnfα and Il1β) and anti-inflammatory genes (Tgfβ1, Il10 and Il6) (FIG. 3D). However, the elevation was greater for pro-inflammatory genes, leading to higher ratios of pro-inflammatory genes: anti-inflammatory genes (Tnfα: Tgfβ1 and Il1β: Il10) for the PIC+RT group compared to the RT alone group (FIG. 3E).

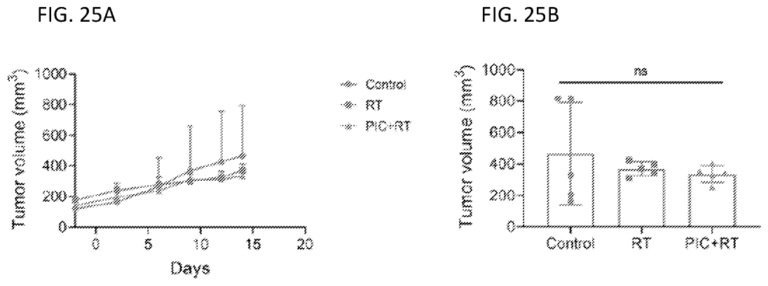

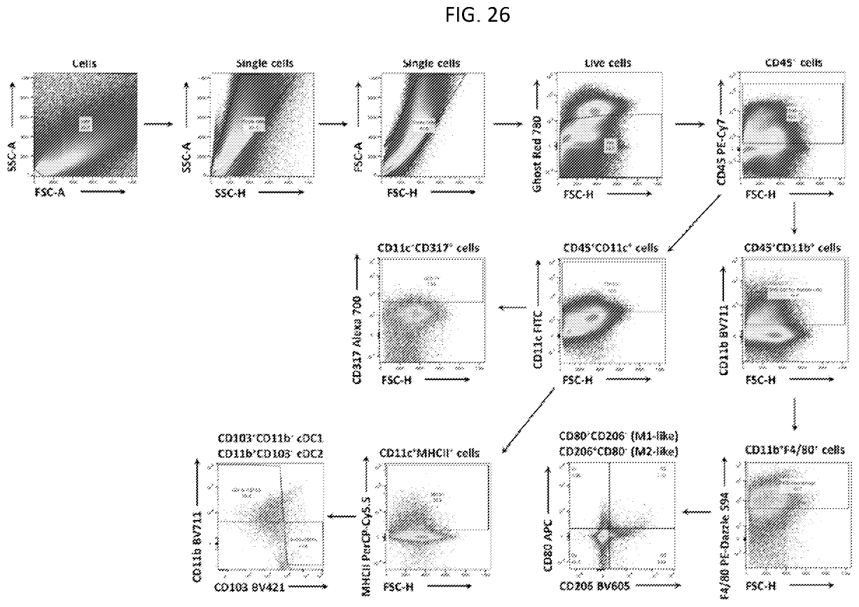

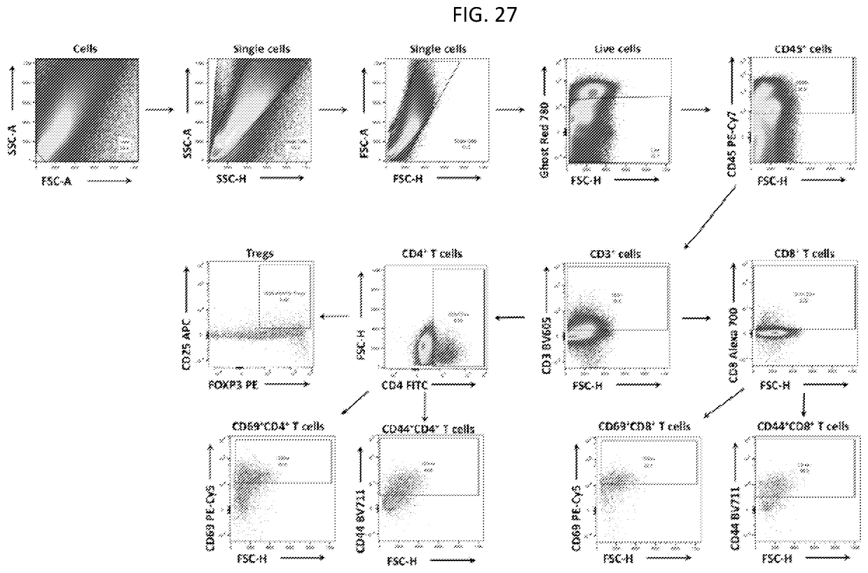

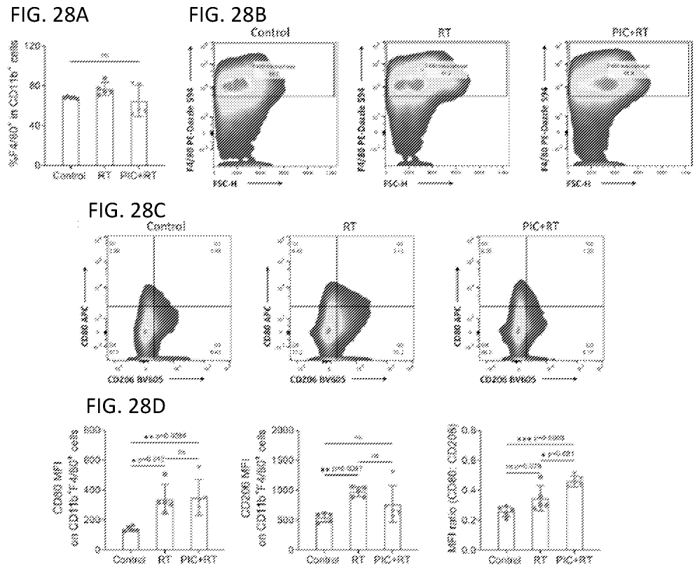

Next, we examined the effects of PIC on the populations of tumor-infiltrating immune cells in radiated B78 melanoma tumors (FIGS. 4A and 25-27). We found that tumor infiltration by TAMs (CD11b⁺F4/80⁺) as a percentage of myeloid cells (CD45⁺CD11b⁺) was not significantly influenced by the RT or PIC+RT treatment (FIGS. 28A-28B), and the frequency of M1 macrophages (CD80⁺CD206⁻) among TAMs in radiated tumors was similar to that in PIC+RT group, with both exhibiting higher percentage of M1 macrophage when compared to untreated control mice (FIGS. 4B and 28C). On the other hand, we observed an elevation in the relative abundance of M2 macrophages (CD206⁺CD80⁻) in tumor following RT alone, but this effect was reduced when RT was combined with PIC injection (FIGS. 4C and 28C). As a result, PIC+RT treatment significantly increased the M1:M2 macrophage ratio when compared to either the untreated control groups or the RT alone group (FIG. 4D). This macrophage polarization effect of PIC on radiated tumors was further verified by the higher expression ratios of CD80:CD206 on TAMs from PIC+RT treated tumors compared to those from tumors treated with RT alone (FIG. 28D).

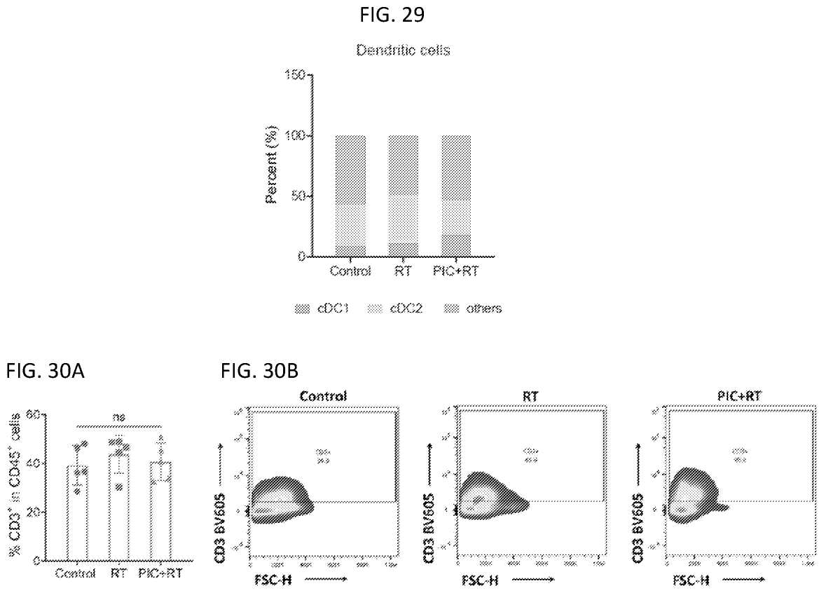

DCs are critical to enable adaptive T cell immunity against cancer cells. cDC1s promote the cross-presentation of tumor antigens to CD8⁺ T cells and are critical for the generation of cytotoxic effector T cell responses, while cDC2s are involved in the activation of T helper type 2 cells, and pDCs highly express TLR-9 and are copious producers of IFN-I when TLR-9 is stimulated. By analyzing the abundance of DC subtypes in the tumor microenvironment, we found that PIC+RT treatment increased the levels of CD103⁺CD11b⁻ cDC1s and decreased the levels of CD11b⁺CD103⁻ cDC2s in B78 melanoma tumors when compared with either untreated control or RT-alone treated tumors (FIGS. 4E-4F). This phenomenon was also observed in the TDLNs, which exhibited a higher level of cDC1s and lower level of cDC2s in the PIC+RT group compared to RT-alone or untreated groups (FIG. 29). RT significantly enhanced the abundance of tumor-infiltrating CD317⁺CD11c⁺ pDCs and the injection of PIC into the radiated tumors increased the percentage of CD80⁺ cells, one of the markers of TLR-9 activation, among pDCs, when compared to the untreated control group FIGS. 4G-4H).

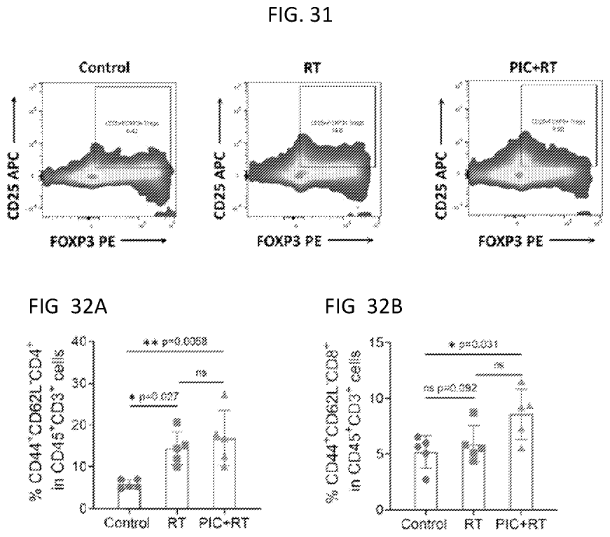

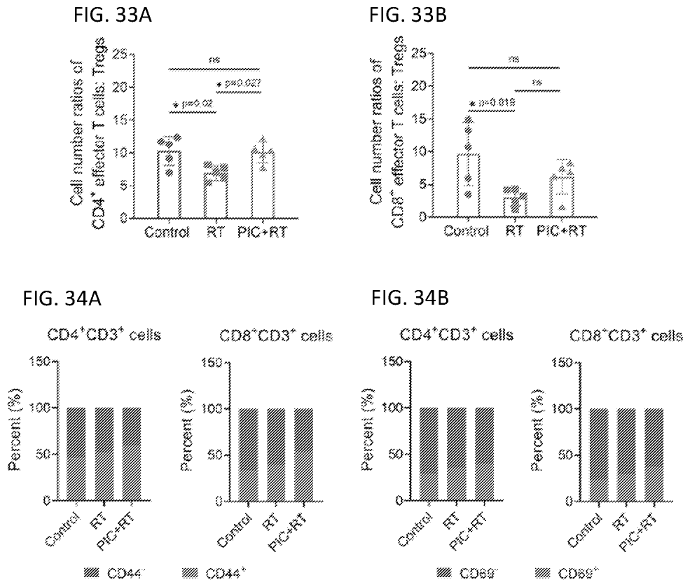

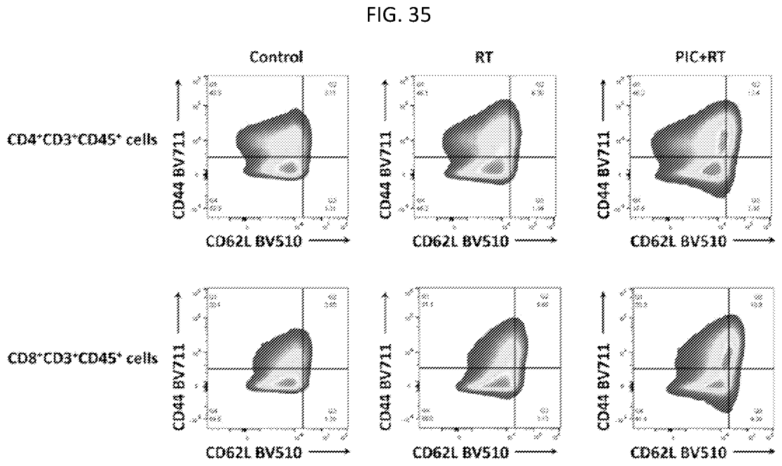

In evaluating tumor infiltration by adaptive immune cells, we observed that RT and PIC+RT treatment did not affect the number of CD3⁺ T cells in tumors (FIGS. 30A-30B), yet PIC+RT treated tumors showed significantly higher infiltration of CD8⁺ T cells compared to tumors treated with RT alone (FIG. 4I). PIC+RT also increased CD4⁺ T cell infiltration of tumors when compared to the control group, although the levels of CD4⁺ T cells infiltration were not significantly different between RT and PIC+RT treated tumors (FIG. 4J). Moreover, RT increased the abundance of CD25⁺FOXP3⁺ Tregs among CD4⁺ T cells, and this effect was antagonized by PIC injection (FIGS. 4K and 31). By analyzing markers of activation state (CD69), effector state (CD44) and memory state (CD62L) on tumor infiltrating T cells, we found higher abundance of activated T cells (CD69⁺CD4⁺ and CD69⁺CD8⁺ cells), effector T cells (CD44⁺CD4⁺ and CD44⁺CD8⁺ cells) and effector memory T cells (CD44⁺CD62L⁻ CD4⁺ and CD44⁺CD62L⁻CD8⁺) out of CD45⁺CD3⁺ T cells among the PIC+RT treated tumors compared to other groups (FIGS. 4L-4M and 32A-32B). RT decreased the ratios of infiltrating CD44⁺CD4⁺ effector T cells: Tregs and CD44⁺CD8⁺ effector T cells: Tregs (FIGS. 33A-33B). However, PIC dampened this immunosuppressive effect of RT (FIGS. 33A-33B). PD-1 is a marker of T cell exhaustion and PD-1/PD-L1 signaling plays a vital role in immune tolerance. Neither RT nor PIC+RT treatment showed any significant effect on the expression of PD-1 on tumor-infiltrating CD4⁺ and CD8⁺ T cells in these B78 tumors (FIGS. 4N-4O). Notably, in addition to these treatment effects in the targeted tumor, we also found increased abundance of central memory T cells (CD44⁺CD62L⁺), CD44⁺ effector T cells and CD69⁺ activated T cells among CD4⁺ and CD8⁺ T cells in the TDLNs in PIC+RT group (FIGS. 4P and 34A-34B, and 35).

### The In Situ Vaccine Effect of RT is Potentiated by PIC.

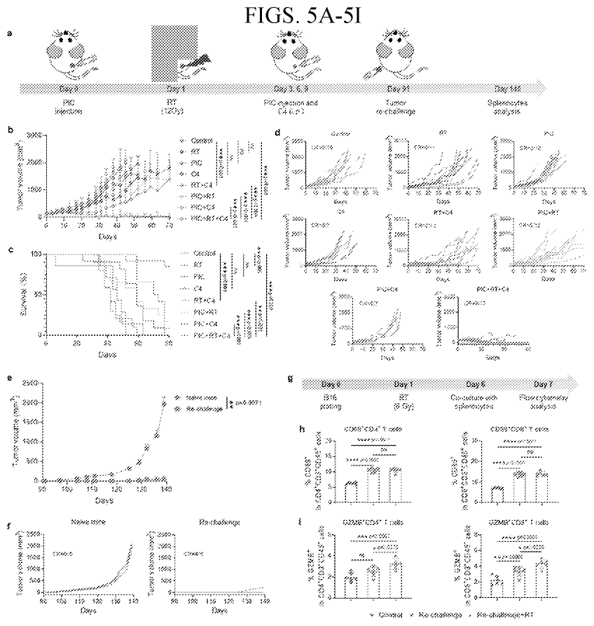

Given the favorable inflammatory and functional effects we observed from the multifunctional PIC in enhancing tumor cell sensitivity to RT, increasing the activation of an IFN-I response by RT, increasing tumor cell infiltration and antigen presentation by DCs, limiting the M2 polarization of macrophages by RT, and augmenting CD8⁺ T cell infiltration of tumor, we examined the potential therapeutic interaction between RT and PIC in vivo. For this, we sought to evaluate the potential cooperative effects of RT and PIC in priming a response to the ICB anti-CTLA-4 in a well-established (4 weeks post implantation), immunologically “cold” B78 melanoma model that does not respond to ICBs alone (FIG. 5A). Mice treated with PIC alone showed tumor growth that was comparable to that in control mice receiving sham treatments (FIGS. 5B-5D) and PIC did not improve response when added to anti-CTLA-4, as compared to anti-CTLA-4 alone. This was not surprising given the design of the PIC as a multifunctional nanoparticle to specifically enhance the in situ vaccine effect of RT. In addition, we observed that PIC did not significantly improve tumor response when combined with RT, as compared to RT alone. This was also expected as we and others have observed that in the absence of ICBs the in situ vaccine effect of RT alone is generally ineffective in activating anti-tumor immunity. When RT and anti-CTLA-4 were combined, B78 tumor growth was reduced, but the response was still limited in this immunologically “cold” melanoma tumor model. Intriguingly, we found that PIC significantly improved the tumor response and animal survival when combined with RT+anti-CTLA-4, compared to RT+anti-CTLA-4 alone (FIGS. 5B-5D).

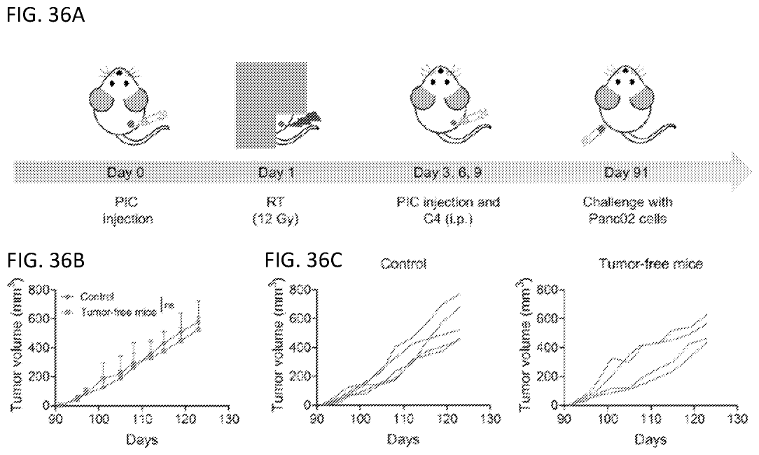

With the triple combination of PIC+RT+anti-CTLA-4, 69.2% (9/13) of mice were rendered tumor-free and all these mice continued to show no evidence of disease at day 90 post-treatment. At day 91 after treatment, we re-challenged a cohort of these mice with a second subcutaneous implantation of the same B78 tumor they had been cured of or an unrelated syngeneic Panc02 tumor that express different TAAs from B78 melanoma in order to assess for tumor-specific immunologic memory. Fifty days later, 80% of these mice (4/5) exhibited no B78 tumor growth and the one mouse developing a tumor showed tumor growth that was significantly slower than that observed in naïve control mice, 100% of which developed B78 tumors (FIG. 5E-5F). However, the disease-free mice re-challenged with Panc02 cells all grew tumors and these exhibited similar growth to that observed Panc02 tumors in naïve control mice (FIGS. 36A-36B). From the B78 melanoma re-challenged mice, we collected splenocytes and co-cultured them with B16 melanoma cells that are parental to B78 and share common TAAs (FIG. 5G). Using flow cytometry on these B16-co-cultured splenocytes, we observed increased levels of early activation marker, CD69⁺, on both CD4⁺ and CD8⁺ T cells from mice rendered disease-free by PIC+RT+anti-CTLA-4, as compared to B16-co-cultured splenocytes from naïve control mice (FIG. 5H). Expression of granzyme B (GZMB), a cytotoxic protein that participates in T-cell killing of tumor cells, was also elevated in CD8⁺ T cells in these splenocytes from mice rendered disease-free by PIC+RT+anti-CTLA-4 (FIG. 5I). This is consistent with the development of tumor-specific immunologic memory following PIC+RT+anti-CTLA-4 treatment and demonstrates the achievement of an enhanced in situ vaccine effect in these mice compared to that achieved with RT+anti-CTLA-4. The PIC+RT+anti-CTLA-4-treated and re-challenged mice survived for more than 300 days from the first treatment without any evidence of disease recurrence, consistent with a curative treatment outcome.

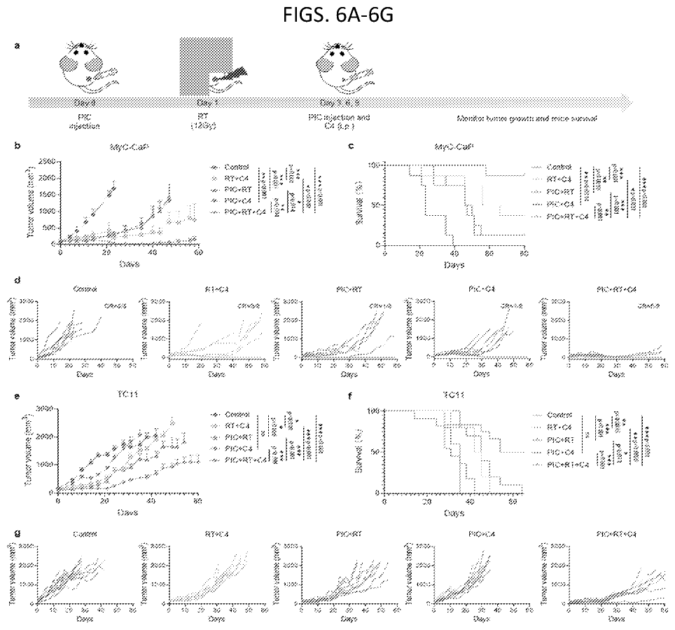

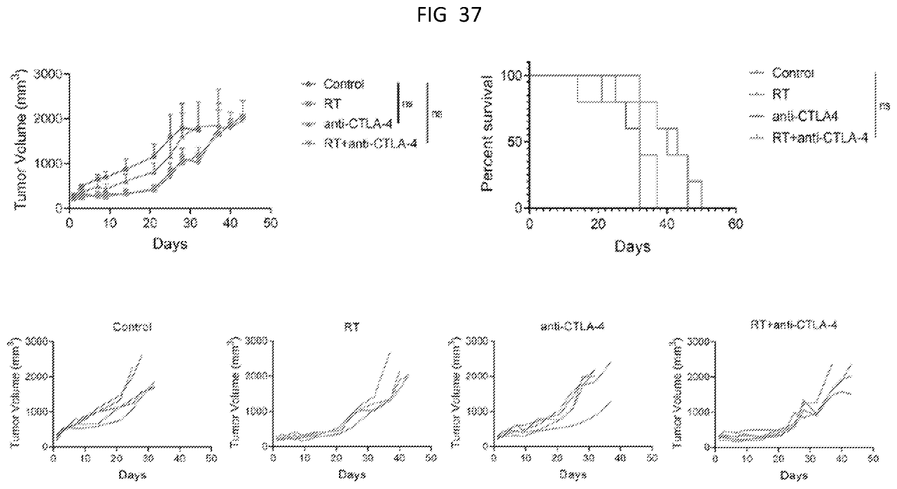

To evaluate the generalizability of our observations, we tested the therapeutic efficacy of the triple combination of PIC+RT+anti-CTLA-4 in the MyC-CaP prostate tumor model in syngeneic male FVB/NTac mice and the orthotopic TC11 breast tumor model in syngeneic female FVB/NTac mice (FIG. 6A). We observed a delay in MyC-CaP tumor growth with the combination of RT+PIC (FIGS. 6B-6D). The combination of PIC+RT+anti-CTLA-4 in this tumor model rendered 62.5% (5/8) of mice disease-free, vs. only 37.5% (3/8) of mice treated with RT+anti-CTLA-4; this was associated with a significant improvement in tumor growth inhibition and overall mice survival (FIGS. 6B-6D). The estrogen-receptor-positive TC11 breast tumor is immunologically “cold” and not responsive to anti-CTLA-4 treatment alone. Even when treated with combined RT and anti-CTLA-4, this TC11 tumor model was poorly responsive (FIG. 37). However, the triple combination of PIC+RT+anti-CTLA-4 significantly suppressed tumor growth in this model and improved overall survival compared to RT+anti-CTLA-4 treatment (FIG. 6E-6G).

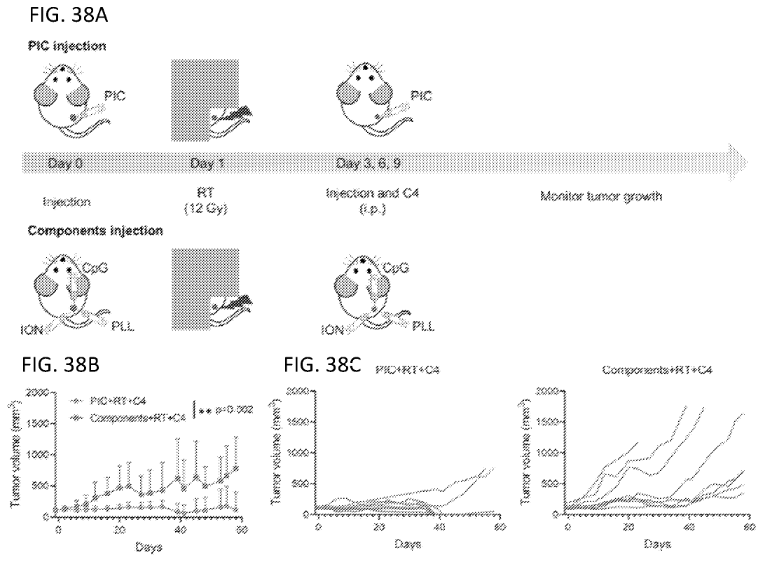

To evaluate the necessity of forming a nanoparticle before intratumoral injection of the PIC components (PLL, ION and CpG) in enhancing the anti-tumor immune response to RT+anti-CTLA-4, we injected the three components (PLL, ION and CpG) separately using a technique in which needle entry was made into three different sides of a single B78 melanoma tumor to minimize the possibility of spontaneously forming PIC nanoparticle in vivo after injection. We evaluated the anti-tumor response when combined with RT and anti-CTLA-4, as compared to the combination of RT and anti-CTLA-4 with fully formed PIC (FIG. 38A). We found the combination of the components with RT and anti-CTLA-4 conferred inferior anti-tumor efficacy, with no mice rendered disease-free at day 60 post-treatment (FIGS. 38B-38C). However, 5/8 mice were rendered disease-free in the PIC+RT+anti-CTLA-4 group (FIGS. 38B-38C). These results indicated that the complexation of the components (PLL, CpG and ION) to form PIC nanoparticle before injection was critical for its multifunctional design.

### PIC+RT+Anti-CTLA-4 Activates an Effective Systemic Anti-Tumor Immune Response.

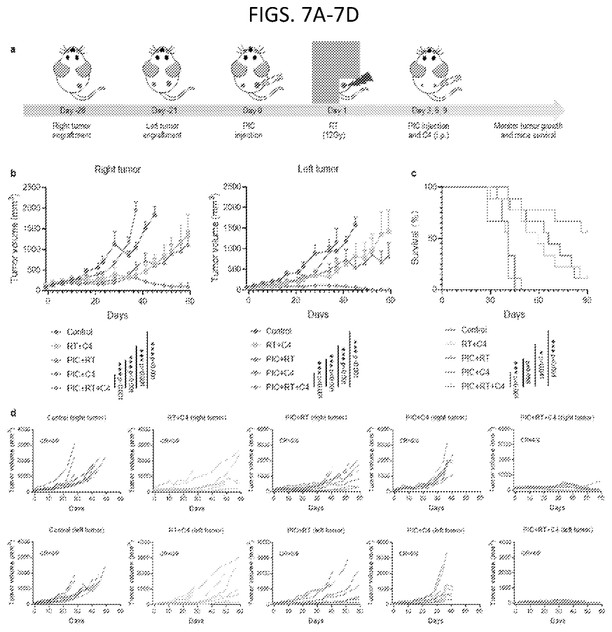

In settings of metastatic disease or circulating tumor cells, it is essential that any in situ vaccine strategy not only activate an effective anti-tumor immune response at a targeted tumor site but also at distant tumor sites elsewhere in the body that are not directly treated with the in situ vaccine regimen. To assess whether local injection of PIC into an RT-treated tumor could improve the systemic anti-tumor immune response when given in conjunction with anti-CTLA-4, we generated mice bearing two B78 melanoma tumors, one on the right flank and the other one implanted 7 days later on the left flank (to simulate a smaller distant site of metastasis). RT and intratumoral injection of PIC were delivered to the larger tumor on the right flank only and the growth of both tumors was monitored (FIG. 7A). For the directly treated right flank tumors, tumor growth was again significantly decreased in the RT+anti-CTLA-4 and the PIC+RT+anti-CTLA-4 groups with the latter exhibiting significantly reduced tumor growth compared to RT+anti-CTLA-4 (FIGS. 7B and 7D). Importantly, these same treatment effects were observed at the left flank tumors, which were not directly treated with RT or injected with PIC. We observed that PIC+RT+anti-CTLA-4 treatment significantly reduced the progression of these left flank tumors compared to all other treatment groups and the triple treatment combination (PIC+RT+anti-CTLA-4) rendered 4/9 mice completely disease-free; no mice in any other group became tumor-free (FIGS. 7B-7D). Moreover, the PIC+RT+anti-CTLA-4 treatment group showed longer mouse survival compared to the RT+anti-CTLA-4 group (FIG. 7C).

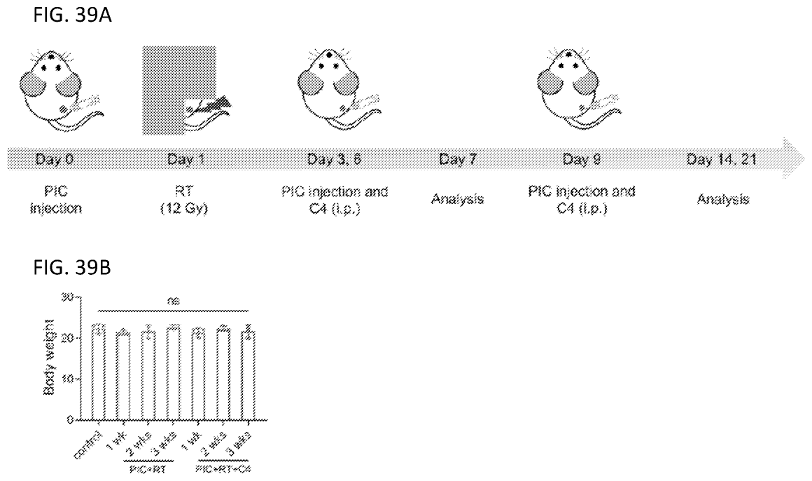

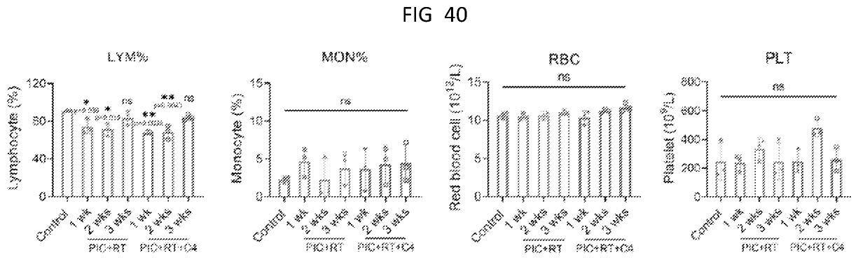

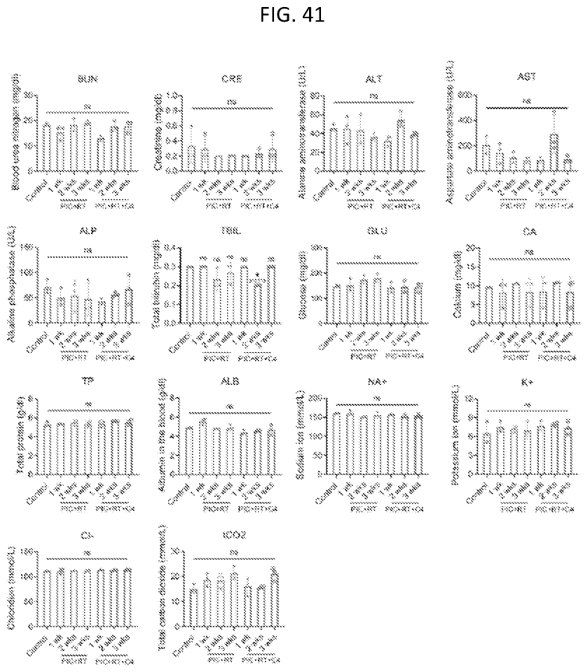

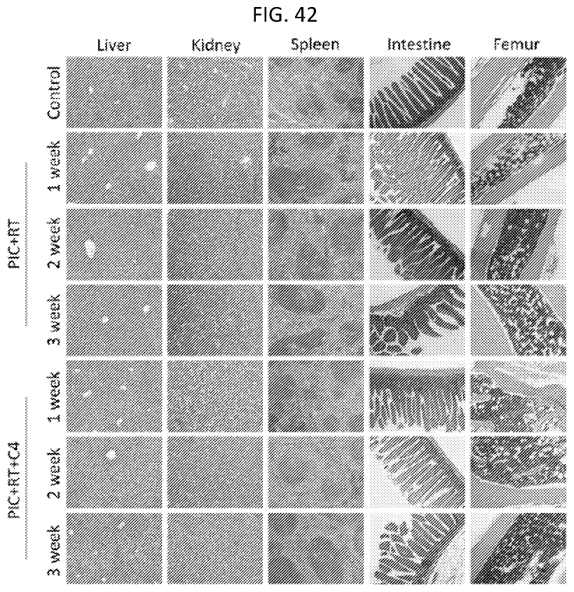

Finally, we evaluated the toxicity of PIC+RT and PIC+RT+anti-CTLA-4 in vivo (FIG. 39a). We did not observe changes in the body weight of mice during treatment (FIG. 39b). By analyzing complete blood counts, we found the percentage of lymphocytes was slightly reduced in the blood of PIC+RT and PIC+RT+anti-CTLA-4 treated mice at 1 week and 2 weeks after RT. At 3 weeks after radiation, the percentage of lymphocytes returned to normal levels (FIG. 40). This transient lymphopenia may have resulted from the radiosensitivity of lymphocytes or from extravasation of these cells in the setting of an activated immune response. Reductions of lymphocytes counts are commonly observed after large field radiation therapy in pre-clinical and clinical studies. The levels of monocytes, red blood cells and platelets were not significantly influenced during or after these treatments (FIG. 40). In addition, no significant variations were found in basic metabolic panels during PIC+RT or PIC+RT+anti-CTLA-4 treatment (FIG. 41). Analysis of normal tissue histology did not reveal any apparent effect of these treatments on liver, kidney, spleen, intestine or bone (FIG. 42).

### DISCUSSION

The positively charged PIC with a hydrodynamic diameter about 110 nm showed internalization in B78 murine melanoma cells. Clonogenic assays and immunofluorescence analysis demonstrated that PIC potentiated the sensitivity of B78 cells to RT (FIGS. 1A-1L). Prior studies suggest that the radiosensitizing effect of IONs can be attributed to the catalytic effects of the released iron ions and the active surfaces of IONs under RT, leading to the generation of ROS in cancer cells. [12, 13] While PIC will also likely enhance the radiosensitivity of tumor infiltrating immune cells, any transient loss of immune cells will be repleted by circulating immune cells drawn into the tumor by the enhanced inflammatory effects of RT+PIC. Notably, PIC accentuated the capacity of RT to activate an IFN-I response in B78 melanoma cells (FIGS. 2A-2J). In addition, PIC directly activated an IFN-I response in pDCs, likely via the stimulation of TLR-9 (FIGS. 2A-2J and 3A-3E). PIC may also contribute to additional mechanisms that lead to the activation of IFN-I response, such as capturing the cell-free DNA released from RT-treated cells and facilitating its internalization in immune cells to stimulate an IFN-I response. Activation of an IFN-I response in tumor and antigen-presenting cells plays an important role in the recruitment and activation of immune cells and has been demonstrated to play a critical role in the capacity of RT to augment response to ICIs. In addition to activating DCs, PIC captured tumor antigens, improved internalization of these in APCs, and enhanced the capacity for generating antigen-specific adaptive T cell immunity (FIGS. 1A-1L and 2A-2J).

Increasing the ratio of M1:M2 polarized macrophages in a tumor may enable more effective development of anti-tumor immune responses. While RT is associated with a potentially detrimental increase in proportion of TAMs that are M2-polarized, in radiated BMDMs we observed that PIC antagonized this effect of RT and thereby increased the ratio of M1:M2 macrophages (FIGS. 2A-2J). Consistent with this, combination of RT and PIC in vivo resulted in elevated bulk tumor mRNA levels of M1-associated Nos2 expression relative to M2-associated Arg1 expression at 15 days after radiation and flow cytometry confirmed increased ratio of TAMs expressing M1 markers versus M2 markers with PIC+RT compared to RT alone (FIGS. 3A-3E and 4A-4P). The combination of RT and PIC also increased the level of effector CD8⁺ T cell infiltration and decreased the level of immunosuppressive regulatory T cells in tumors relative to RT alone (FIGS. 4A-4P). These results demonstrate the capacity of the PIC to favorably immunomodulate the radiated tumor microenvironment.

When combined with anti-CTLA-4, the PIC+RT in situ vaccination enabled greater tumor response and improved survival as well as tumor-specific immune memory and robust systemic anti-tumor immunity at tumors not directly treated by RT or PIC injection (FIGS. 5a-5i and 7a-7d). The effects of RT+PIC were broadly recapitulated in two additional difficult to treat, immunologically “cold” tumor models of prostate and breast cancer using a distinct mouse strain (FIGS. 6A-6G). These results demonstrate that the potentiation effect of PIC on the in situ vaccination of RT can be applied in diverse tumor settings and can prime a potent systemic anti-tumor immune response when combined with anti-CTLA-4 for the treatment of metastatic disease. Intratumoral injection of PIC minimizes its potential for triggering systemic toxicity. Consistent with this, in mice treated with PIC+RT or PIC+RT+anti-CTLA-4 we observed no evidence of hepatic, renal, gastrointestinal, or autoimmune toxicities and modest hematological effects that did not appear to result in symptoms or adverse effects (FIGS. 40-41).

### Example 4-In Situ Vaccination of Tumors with RT, IL2 and PIC NP

### Methods

Cell culture and animals. B78 (B78-D14, GD2+) melanoma cells were originated from B16 cells and obtained from Ralph Reisfeld (Scripps Research Institute). B16 melanoma cells were obtained from Memorial Sloan Kettering Cancer Center. MOC2 head and neck cancer cells were generously provided by Dr. Ravindra Uppaluri (Dana-Farber Cancer Institute). All animal studies in this research were approved by the Institutional Animal Care and Use Committee at the University of Wisconsin-Madison (protocol: M005670). All mice (C57BL/6, 7-8 weeks, male and female) were purchased from Taconic. To establish tumor-bearing mice, mice were intradermally injected with tumor cells (B78 melanoma model: 2×10⁶ cells on the right flanks; B78 melanoma two-tumor model: 2×10⁶ cells on the right flanks, and 1 week later 2×10⁶ cells engrafted on left flanks; MOC2 head and neck tumor model: 2×10⁶ cell on the right flanks). Once the tumor volumes (primary tumor volumes for the two-tumor bearing mice) were about 100 mm³, mice were randomized for the initiation of treatment. For the two-tumor bearing mice, the treatment was performed only on the right flank (primary) tumors. Tumors were measured twice weekly for at least 40 days unless mice died or were euthanized because of large tumor size, tumor necrosis, or evidence of pain or distress. Tumor diameters were measured with a Vernier caliper, and tumor volume was calculated through the equation: tumor volume-longer diameter×shorter diameter²×0.5.

In vitro co-culture studies. B78 cells were seeded in 6-well plates with 0.25×10⁶/well, and were radiated with a RS225 Cell Irradiator (Xstrahl) at a dose of 12Gy after culturing for 24 hours. Fresh culture media was exchanged one hour after the radiation. After culturing for another 4 days, splenocytes were collected from naïve C57BL/6 mice and added to the B78 cells with 4×10⁶/well, and PIC and IL2 were added to the co-cultures at a concentration of 0.5 g/mL and 67 IU/mL, respectively. 24 hours later, the splenocytes were collected for flow cytometry and RT-qPCR analyses. To study the direct impact of PIC+IL2 to the splenocytes, splenocytes were collected from the naïve C57BL/6 mice and seeded in 6-well plates with 4×10⁶/well. PIC and IL2 were added to the cells at a concentration of 0.5 μg/mL and 67 IU/mL, respectively. 24 hours later, the splenocytes were collected for flow cytometry and RT-qPCR analyses.

For flow cytometry analyses, the cells were stained with GhostRed 780, followed by incubating with CD16/CD32 (Fc block) for 5 minutes. Then, the cells were stained with anti-CD45 PE-Cy7, anti-CD3 BV510, anti-CD4 FITC, anti-CD8a Alexa 700, anti-NK1.1 PE-Cy5, anti-CD69 BV421, anti-CD44 BV605, and anti-CD25 APC. After the cells were fixed and permeabilized, they were stained with anti-FOXP3 PE. Flow cytometry was performed on an Attune Cytometer (ThermoFisher), and UltraComp Beads eBeads (Invitrogen) were used for compensation.

For RT-qPCR analyses, the cells were washed with cold PBS, and then 1 mL Trizol Reagent (Ambion) was added. RNA was isolated using RNeasy Mini Kit (QIAGEN, Cat: 74106) according to the manufacturer's instructions. cDNA was synthesized using QuantiTect Reverse Transcription Kit (QIAGEN, Cat: 205314) according to the manufacturer's standard protocol. Quantitative polymerase chain reaction (RT-qPCR) was performed using Taqman Fast Advanced Mix and Predesigned Taqman gene expression assays for Ifnγ, Il4, Fas, Nos2, Arg1 and Il1α. Thermal cycling conditions (QuantStudio 6, Applied Biosystem) included the UNG incubation stage at 50° C. for 2 minutes, followed by AmpliTaq™ Fast DNA polymerase activation stage at 95° C. for 2 minutes and 40 cycles of each PCR step (denaturation) 95° C. for 1 second and (annealing/extension) 60° C. for 20 seconds. For data analyses, Ct values were transferred to an Excel file and fold change was determined using the ΔΔCt method. Hprt was used as the endogenous control.

Treatment. For PIC+RT, RT+IL2 and PIC+RT+IL2 groups, PIC (1.4 mg/mL, 100 mL) was intratumorally injected on days 0, 3, 6 and 9. Radiation with a dose of 12Gy was delivered to the tumors on day 1 using an XPad 320 cabinet irradiator (Precision X-Ray, Inc). IL2 (75000 IU, 100 mL) was intratumorally injected daily on days 6-10. For PIC₀₋₆+RT, RT+IL2₀₊₆ and (PIC+IL2)₀₊₆+RT, PIC (5.6 mg/mL, 100 mL) was intratumorally injected on days 0 and 6. Radiation with a dose of 12Gy was delivered to the tumors on day 1 using an XPad 320 cabinet irradiator (Precision X-Ray, Inc). IL2 (75000 IU, 30 mL) was intratumorally injected daily on days 0 and 6. For (PIC+IL2)₀+RT, PIC (5.6 mg/mL, 100 mL) was intratumorally injected on day 0. Radiation with a dose of 12Gy was delivered to the tumors on day 1 using an XPad 320 cabinet irradiator (Precision X-Ray, Inc). IL2 (75000 IU, 30 mL) was intratumorally injected daily on day 0.

Flow cytometry analysis of tumors. Tumor tissues were collected at day 15 after the initiation of treatment from the B78 two-tumor bearing mice. The samples were enzymatically dissociated with DNase and collagenase on a Gentle MACS Octodissociator (Miltenyi Biotec) and then filtered through a 70 mm cell strainer and red blood cells were lysed using RBS lysis buffer. The single cell suspensions were divided for innate immune cells staining and adaptive immune cells staining separately. For innate immune cells staining, the cells were stained with GhostRed 780, followed by incubating with CD16/CD32 (Fc block) for 5 minutes. Then, the cells were stained with anti-CD45 PE-Cy7, anti-CD11b BV711, anti-F4/80 PE-Dazzle 594, anti-CD80 PE, anti-CD206 APC, anti-CD11c FITC, anti-MHCII BV510. For adaptive immune cells staining, the cells were stained with GhostRed 780, followed by incubating with CD16/CD32 (Fc block) for 5 minutes. Then, the cells were stained with anti-CD45 PE-Cy7, anti-CD3 BV510, anti-CD4 FITC, anti-CD8a Alexa 700, anti-NK1.1 PE-Cy5, anti-CD69 PerCP-Cy5.5 and anti-CD25 APC. After the cells were fixed and permeabilized, they were stained with anti-FOXP3 PE. Flow cytometry was performed on an Attune Cytometer (ThermoFisher), and UltraComp Beads eBeads (Invitrogen) were used for compensation.

Flow cytometry analysis of tumor-draining lymph nodes (TDLNs). TDLNs were collected at day 15 after the initiation of treatment from the B78 two-tumor bearing mice. The samples were enzymatically dissociated with DNase and collagenase on a Gentle MACS Octodissociator (Miltenyi Biotec) and then filtered through a 70 μm cell strainer. The red blood cells were lysed using RBS lysis buffer. For innate immune cells staining, the cells were stained with GhostRed 780, followed by incubating with CD16/CD32 (Fc block) for 5 minutes. Then, the cells were stained with anti-CD45 PE-Cy7, anti-CD11b BV711, anti-F4/80 PE-Dazzle 594, anti-CD80 PE, anti-CD206 APC, anti-CD11c PerCP-Cy5.5, anti-MHCII BV510. For adaptive immune cells staining, the cells were stained with GhostRed 780, followed by incubating with CD16/CD32 (Fc block) for 5 minutes. Then, the cells were stained with anti-CD45 PE-Cy7, anti-CD3 BV510, anti-CD4 FITC, anti-CD8a Alexa 700, anti-CD69 BV421, anti-CD44 PE and anti-CD62L PE-Cy5. Flow cytometry was performed on an Attune Cytometer (ThermoFisher), and UltraComp Beads eBeads (Invitrogen) were used for compensation.

Flow cytometry analysis of blood. Blood was collected at day 15 after the initiation of treatment from the B78 two-tumor bearing mice. The red blood cells were lysed using RBS lysis buffer. The cells were stained with GhostRed 780, followed by incubating with CD16/CD32 (Fc block) for 5 minutes. Then, the cells were stained with anti-CD45 PE-Cy7, anti-CD3 BV510, anti-CD4 FITC, anti-CD8a Alexa 700, anti-PD-1 BV421, anti-CD44 PE, anti-NKG2D PE-Dazzle 594. Flow cytometry was performed on an Attune Cytometer (ThermoFisher), and UltraComp Beads eBeads (Invitrogen) were used for compensation.

qPCR analysis of tumors. Tumor tissues were collected at day 15 after the initiation of treatment from the B78 two-tumor bearing mice. Tumor tissues were homogenize using a Bead Mill Homogenizer (Bead Ruptor Elite, Omni International). RNA was isolated using RNeasy Mini Kit (QIAGEN, Cat: 74106) according to the manufacturer's instructions. cDNA was synthesized using QuantiTect Reverse Transcription Kit (QIAGEN, Cat: 205314) according to the standard protocol. Quantitative polymerase chain reaction (RT-qPCR) was performed using Taqman Fast Advanced Master Mix and predesigned Taqman gene expression assays for Ifng, Il4, Ifnb, Il1α and Fas. Thermal cycling conditions and data analysis were performed the same as indicated above. Hprt was used as the endogenous control.

Tumor re-challenge. At day 60 after the initiation of treatment, tumor-free mice obtained from the PIC+RT+IL2 treatment group in B78 melanoma model were re-challenged by an intradermal engraftment of 2×10⁶ B78 melanoma cells in the left flank. A group of age-matched naïve mice were also engrafted with 2×10⁶ B78 melanoma cells for tumor growth as control. Tumor growth was monitored for another 60 days.

Evaluation of immune memory. B16 cells were seeded in 12-well plates with 2×10⁺/well, and were radiated with a RS225 Cell Irradiator (Xstrahl) at a dose of 12Gy after culturing for 24 hours. Fresh culture media was exchanged one hour after the radiation. After culturing for another 4 days, splenocytes were collected from the tumor-free mice those rendered by the PIC+RT+IL2 treatment and added to the B16 cells with 2×10⁶/well. 24 hours later, the splenocytes were collected for flow cytometry and RT-qPCR analyses.

For flow cytometry analyses, the cells were stained with GhostRed 780, followed by incubating with CD16/CD32 (Fc block) for 5 minutes. Then, the cells were stained with anti-CD45 PE-Cy7, anti-CD3 BV510, anti-CD4 FITC, anti-CD8a Alexa 700, anti-CD69 PE, and anti-CD44 BV605. Flow cytometry was performed on an Attune Cytometer (ThermoFisher), and UltraComp Beads eBeads (Invitrogen) were used for compensation.

For RT-qPCR analyses, the cells were washed with cold PBS, and then 1 mL Trizol Reagent (Ambion) was added. RNA was isolated using RNeasy Mini Kit (QIAGEN, Cat: 74106) according to the manufacturer's instructions. cDNA was synthesized using QuantiTect Reverse Transcription Kit (QIAGEN, Cat: 205314) according to the manufacturer's standard protocol. Quantitative polymerase chain reaction (RT-qPCR) was performed using Taqman Fast Advanced Mix and Predesigned Taqman gene expression assays for Ifnγ and Il2. Thermal cycling conditions and data analysis were performed the same as indicated above. Hprt was used as the endogenous control.

Immunohistochemistry. Tumor tissues were collected at day 15 after the initiation of treatment from the B78 two-tumor bearing mice. The tumor samples were embedded in paraffin and the blocks were sectioned into 50□m slices. The antibodies used for IHC included: CD4 Monoclonal Antibody (Invitrogen, Cat: 14-9766-37. Dilution: 1:500), CD8a Monoclonal Antibody (Invitrogen, Cat: 14-0808-82. Dilution: 1: 1000). Standard IHC methods were performed as previously described.^{9, 42} The samples those stained without the primary antibody served as negative controls.

Biodistribution. Cy5 labeled PLL (Cy5-PLL) was synthesized as previously described.¹⁶ Cy5 labeled PIC (Cy5-PIC) was prepared through the complexation between Cy5-PLL, ION and CpG as mentioned above. Cy5-PIC (200 μL, 560 μg) was intratumorally injected to the right tumors of B78 melanoma two-tumor bearing mice when the volume of right tumors was about 200 mm³ at days 0 and 6. At day 15, the mice were euthanized, and tumors and tumor-draining lymph nodes were collected for imaging.

Statistics. Prism 8 (GraphPad Software) was used for statistical analyses. One-way ANOVA was used for the gene expression in vitro and in vivo, and the flow cytometry data in vitro and in vivo. Linear mixed effects modeling was used for all the tumor growth data, and log-rank test was used for all the mice survival data.

### Results

### PIC and IL2 Increase the Expression of Pro-Inflammatory Cytokines and Augment T Cell Activation.

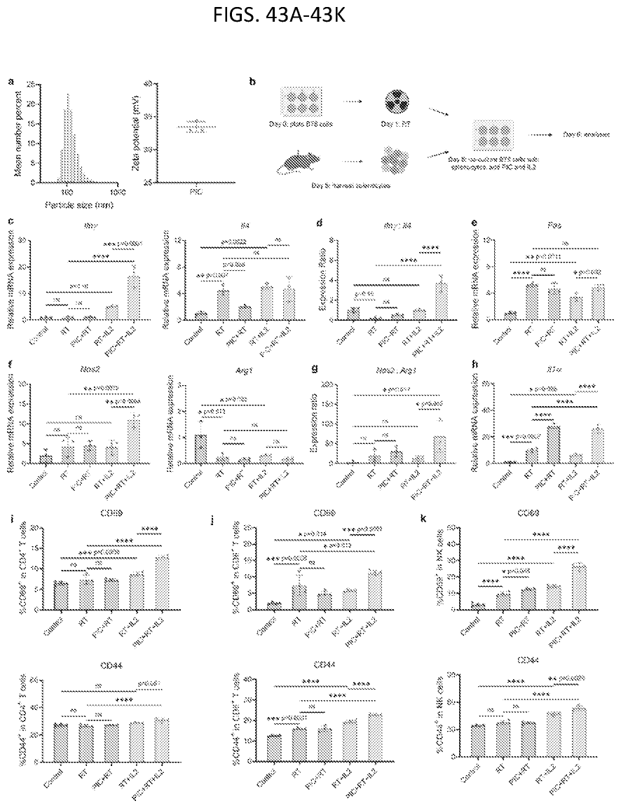

We evaluated the effects of PIC and IL2 on T cell activation and cytokine expression in vitro. We co-cultured radiated B78 melanoma cells with splenocytes collected from naïve C57BL/6 mice and treated these co-cultures with PIC, IL2 or their combination (FIG. 43B). By analyzing the mRNA expression of cytokines in splenocytes through RT-qPCR, we found PIC significantly enhanced the expression of Ifnγ when it was added to the IL2-treated radiated co-cultures, although it did not show any direct effect on the radiated co-cultures in the absence of IL2 (FIG. 43C). We also found that RT significantly increased the expression of Il4, a marker of Th2 response, and this effect was weakened by addition of PIC to co-culture, although IL2 and PIC+IL2 treatments did not influence this RT-induced Il4 expression. By comparing the expression ratios of Ifnγ: Il4, we found that PIC promoted Th1 polarization when combined with IL2 (FIG. 43D).

Fas and Fas ligand (FasL) interactions are involved in immune-mediated tumor cell killing via activation of extrinsic apoptosis. We observed higher Fas expression in the splenocytes when the radiated co-cultures were treated with PIC+IL2 compared to those treated with IL2 alone (FIG. 43E). As shown in FIG. 43F-43G, and consistent with the tests of using PIC alone, in the presence of IL2, the addition of PIC treatment promoted M1 macrophage polarization, leading to a higher ratio in the expression of Nos2:Arg1 among splenocytes, markers of M1 and M2 macrophages, respectively. Higher expression of pro-inflammatory cytokine, Il1α, was also observed when PIC treatment was added to the co-cultures (FIG. 43H).

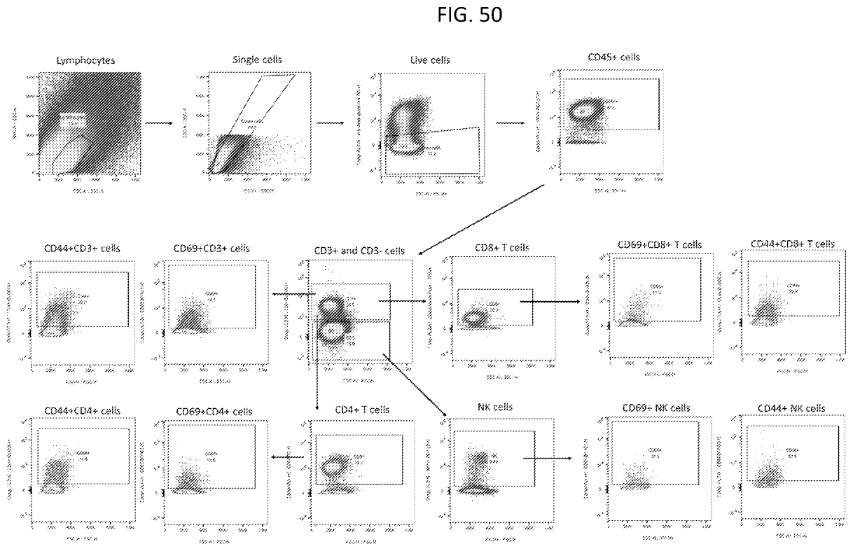

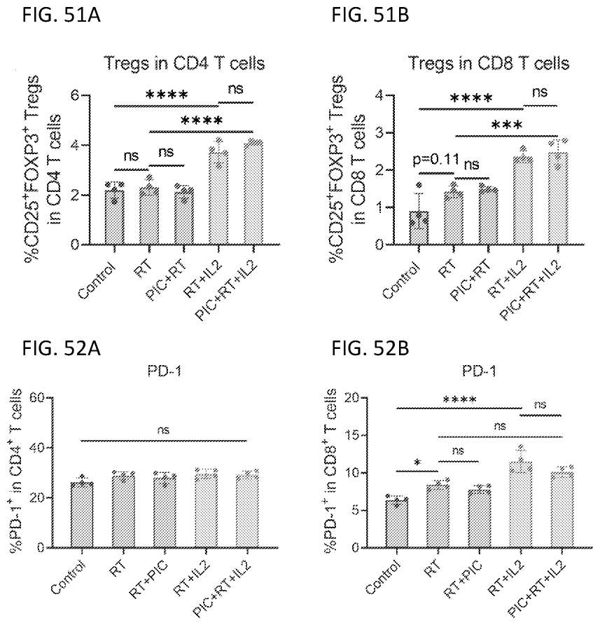

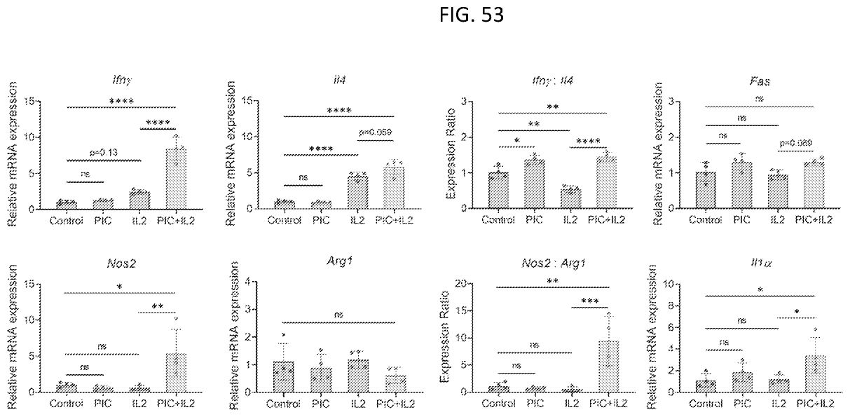

Since IL2 can play a crucial role in modulating the functions of T cells and NK cells, we subsequently evaluated the effects of PIC on the activation state (CD69) and effector state (CD44) of T cells and NK cells when it was combined with IL2 (FIG. 50). In FIG. 43I-43J, the abundance of activated T cells (CD69⁺) and effector T cells (CD44⁺) significantly increased among both CD4⁺ T cells and CD8⁺ T cells when PIC was added to the IL2-treated radiated co-cultures, although this effect of PIC was not observed in the absence of IL2. For NK cells, PIC did not influence the CD44⁺ proportion but slightly increased the CD69⁺ proportion, and these effects were significantly amplified in the presence of IL2 (FIG. 43K). By analyzing the regulatory T cells (Tregs), we found IL2 treatment significantly increased the levels of CD25⁺FOXP3⁺ Tregs, while PIC showed negligible effects on Treg levels at the tested concentration (FIGS. 51A-51B). Although RT+IL2 increased the PD-1 expression on CD8⁺ T cells, PIC did not exhibit significant effects on PD-1 expression on either CD4⁺ or CD8⁺ T cells (FIGS. 52A-52B).

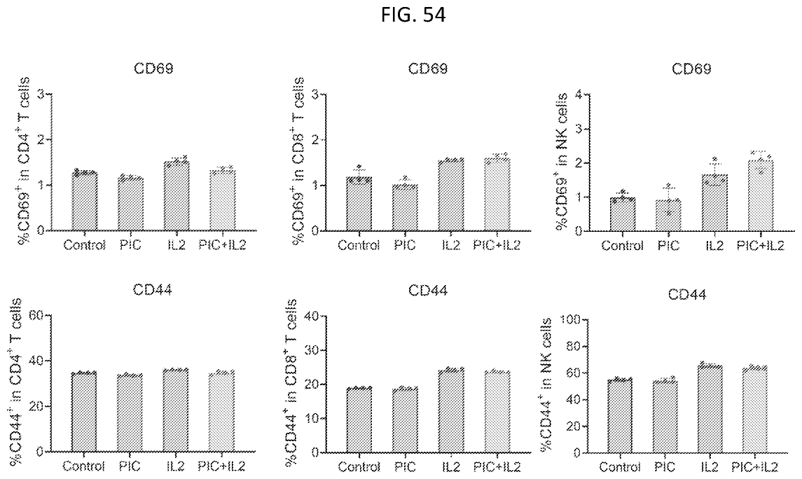

To determine whether the effects of PIC and PIC+IL2 on the splenocyte cytokine expression and T cell activation were direct effects of these therapies on immune cells, we treated splenocytes with PIC, IL2 or PIC+IL2 in the absence of radiated tumor cells. Similar trends were found in the patterns of cytokine expression when PIC was added to IL2-treated splenocytes as compared to those observed in the splenocyte-radiated tumor cell co-culture system, leading to higher expression ratios of Ifnγ. Il4 and Nos2:Arg1 (FIG. 53). This indicated that PIC+IL2 could directly regulate the cytokine expression of immune cells and promote a favorable Th1 and M1 polarization of T cells and macrophages in the TME. When added to IL2, however, PIC did not affect the activation state (CD69⁺) or effector state (CD44⁺) of T cells or NK cells in the absence of radiated tumor cells (FIG. 54). This suggested a critical role for radiated tumor cells in promoting activation of T cells and NK cells, even in the presence of PIC+IL2.

### PIC Enhances the Systemic Anti-Tumor Immune Response to RT+IL2 Resulting in Immune Memory.

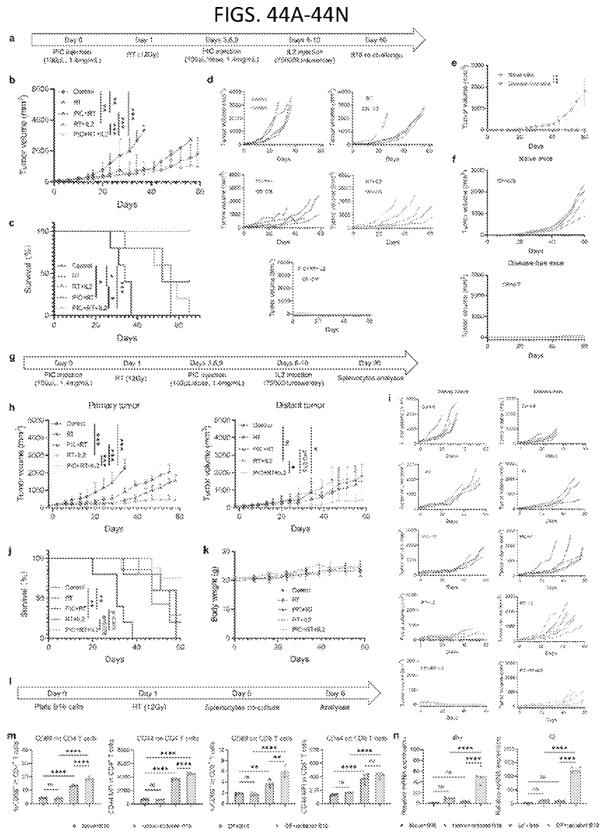

Given the coordination observed in vitro between PIC and IL2 in activating T cells and promoting the M1 and Th1 polarization of macrophages and T cells, we investigated the potential effects of PIC on the anti-tumor immunity of RT+IL2 in the immunologically “cold” B78 melanoma model. We injected IL2 to the radiated tumors on days 6 to 10 because our previous work demonstrated that the IL2-conjugates injected using this schedule was optimal for priming an anti-tumor T cell immune response after RT. PIC was injected on days 0, 3, 6 and 9 in accordance with our prior studies (FIG. 44A). As shown in FIGS. 44B-44D, both PIC+RT and RT+IL2 treatments delayed the B78 melanoma growth and prolonged mouse survival, while these effects were further improved in the triple combinational PIC+RT+IL2 treatment group, resulting in 100% (7/7) mice tumor-free at day 60 following the initial treatment. These disease-free mice obtained after PIC+RT+IL2 treatment exhibited resistance to a second B78 re-challenge, with 6/7 of them not showing tumor development after re-challenge and 1/7 showing tumor growth but much slower than that on naïve mice. (FIG. 44E-44F).

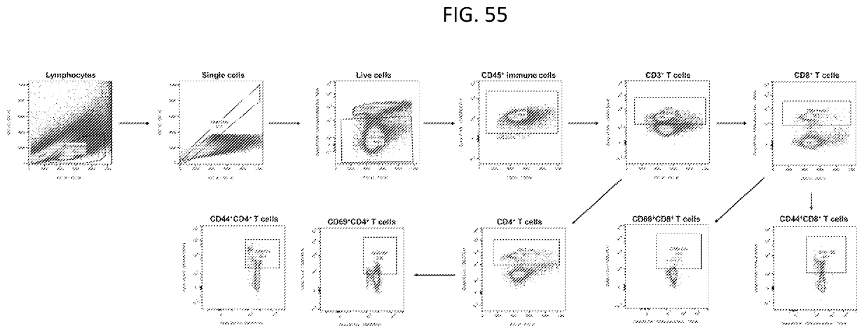

To assess systemic anti-tumor immunity resulting from PIC+RT+IL2 treatment, we implanted C57BL/6 mice with two B78 tumors, the first on the right flank and the second on the left flank one week later to mimic a tumor metastasis. Only the right tumors received the triple treatment of PIC+RT+IL2, and the growth of both tumors was monitored (FIG. 44G). As shown in FIGS. 44H-44J, PIC+RT+IL2 not only effectively eradicated the primary tumors, but also significantly inhibited the progression of distant tumors, and prolonged the survival of mice, when compared to the PIC+RT and RT+IL2 treatments. The mice showed negligible changes in body weight during the study (FIG. 44K). At day 90 after the initiation of treatment, we collected splenocytes from the disease-free mice obtained after PIC+RT+IL2 treatment and co-cultured them with B16 melanoma cells to assess immune memory (FIGS. 44L and 55). Using flow cytometry, we found that the expression of CD69 and CD44 on both CD4⁺ T cells and CD8⁺ T cells from disease-free mice was significantly higher compared to that on T cells from naïve control mice after co-cultured with B16 cells (FIG. 44M). When the B16 cells in the co-cultures were pre-radiated, the upregulation of CD69 on CD4⁻ T cells and CD8⁻ T cells in the splenocytes of disease-free mice was further increased, which may be related to the enhanced immunogenicity of radiated tumor cells and/or the production of type I interferon by tumor cells following radiation. Gene expression profiling of splenocytes in this co-culture (FIG. 44N) demonstrated increased expression of Ifng and Il2 in the splenocytes from the disease-free mice compared to those from naïve mice after co-culture, further confirming the generation of immune memory effects.

### Optimizing PIC+RT+IL2 Dosing for Clinical Translation

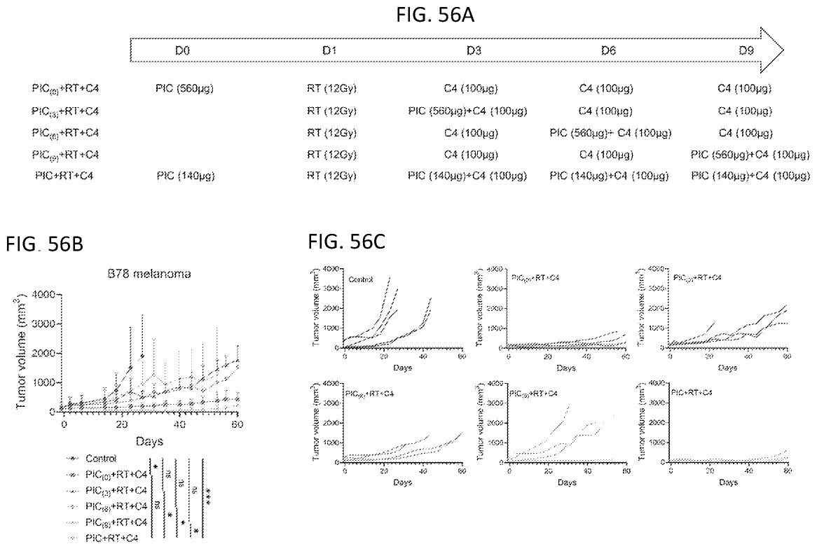

Although PIC+IL2 markedly improved the in situ vaccine effect of RT, the translational potential of this strategy could be limited by a requirement for multiple intratumoral injections. To ameliorate this, we tested whether comparable efficacy could be achieved by combining the four doses of PIC into a single injection and tested the effect of this single dosing approach on the in situ vaccination of RT at different timepoints (injection on day 0, 3, 6 or 9 after RT) (FIG. 56a). We sought to separately evaluate the optimal timing of PIC and IL2 injections relative to the timing of RT. As we previously reported however, RT+PIC has minimal efficacy on its own, so to enable an efficacy signal in this timing and dosing study, we combined RT+PIC with anti-CTLA-4, a regimen that we previously demonstrated to have anti-tumor response in the B78 melanoma model.¹⁶ We found that PIC injection at day 0 (one day prior to RT) achieved similar tumor growth inhibition when compared to mice receiving multiple PIC injections (days 0, 3, 6 and 9) when combined with the immune checkpoint inhibitor anti-CTLA-4 (FIGS. 56b-56c). This is consistent with our prior observation that intratumorally injected PIC is retained at tumor sites for many days¹⁶, and suggests that multiple PIC injections after RT may be less necessary.

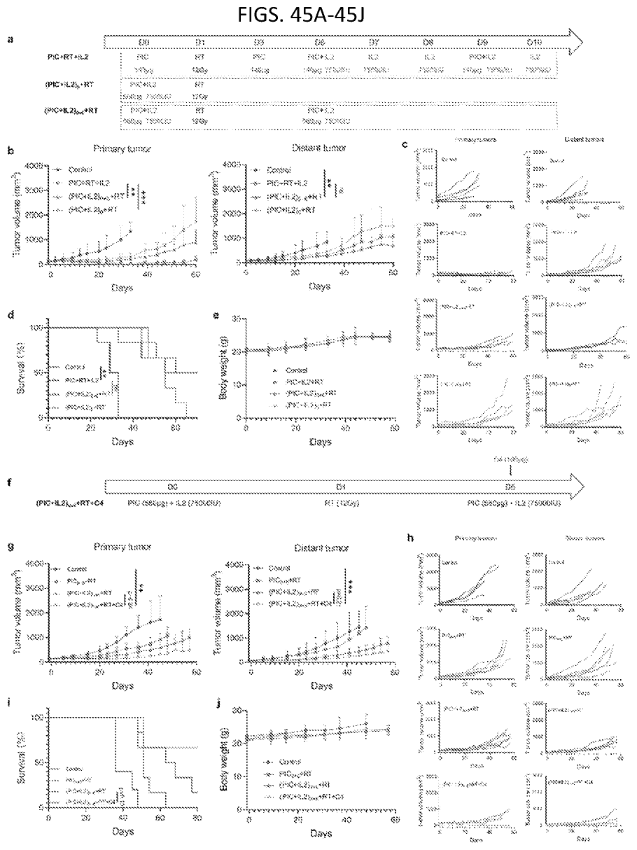

Our previous work indicated that days 6 to 10 after RT may be a favorable timeframe for the injection of IL2-conjugates to a radiated TME in order to strengthen the in situ vaccine effect. To begin evaluating an optimal approach to IL2 administration using fewer injections in the context of RT+PIC, we combined PIC with IL2 and evaluated the injection on days 0 and 6. The anti-tumor and abscopal effects of RT+PIC+IL2 were tested on mice bearing two B78 tumors. We compared our original PIC+RT+IL2 treatment regimen (PIC injection at day 0, 3, 6, 9; IL2 injection at days 6-10 daily) with two reduced injection regimens (PIC+IL2 injection on day 0, or day 0+6) (FIG. 45A). (PIC+IL2)₀₊₆+RT treatment was more effective than (PIC+IL2)₀+RT with regards to inhibiting the growth of the radiation-targeted primary tumors and both regimens were less effective than our original multi-injection PIC+RT+IL2 treatment. However, with regards to the inhibition of distant tumor growth we observed comparable efficacy for (PIC+IL2)₀₊₆+RT compared to the original multi-injection PIC+RT+IL2 regimen (FIGS. 45B-45C). The survival curves for these treatments (FIG. 45D) demonstrated a similar survival rate for mice receiving (PIC+IL2)₀₊₆+RT and the original PIC+RT+IL2 treatments with no significant changes in animal weight noted between these regimens (FIG. 45E). Although B78 melanomas are immunologically “cold” and poorly responsive to ICIs, we found that the (PIC+IL2)₀₊₆+RT treatment sensitized B78 melanomas to a single injection of anti-CTLA-4 and the combination of (PIC+IL2)₀₊₆+RT with anti-CTLA-4 improved anti-tumor efficacy and survival of mice bearing two B78 melanoma tumors (FIGS. 45F-45I). No significant change was observed in mouse body weight when combining RT+ (PIC+IL2)₀₊₆ with anti-CTLA-4 (FIG. 45J).

### PIC and IL2 Immunomodulate the Radiated TME and Stimulate Immune Response at Distant Tumors

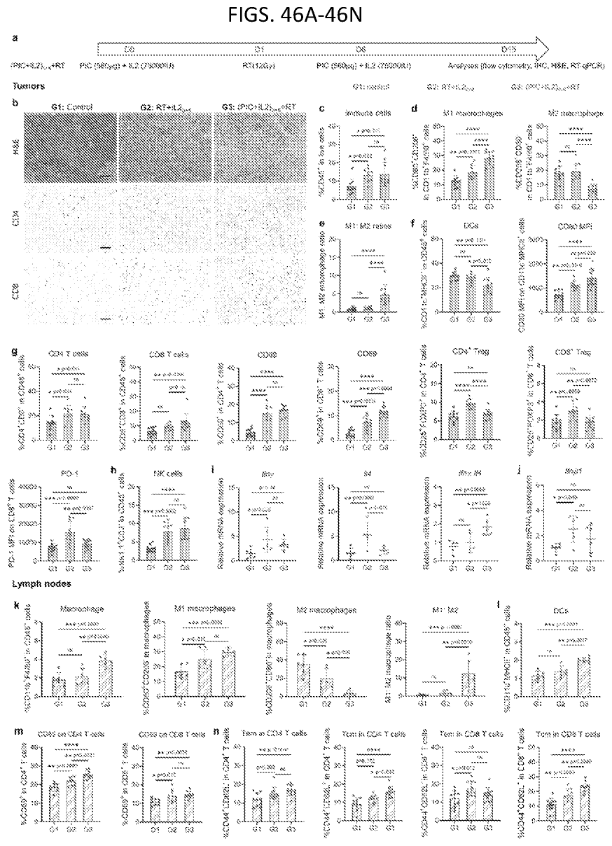

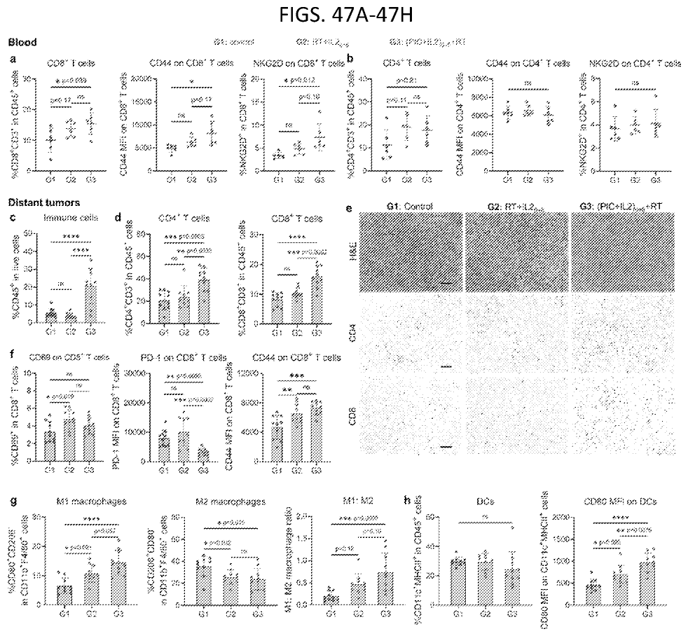

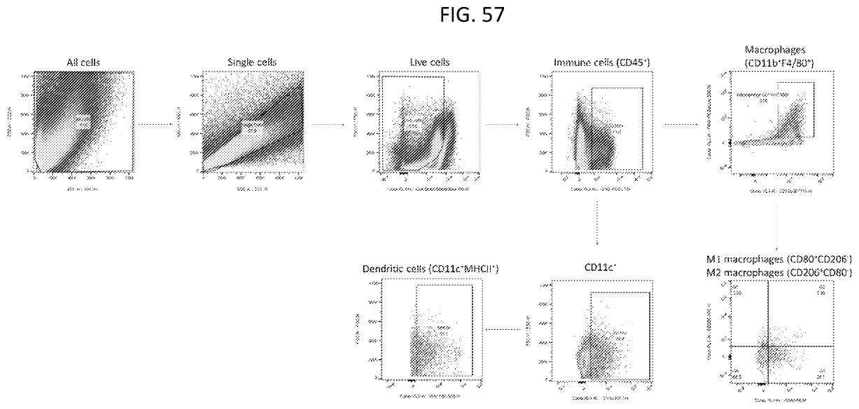

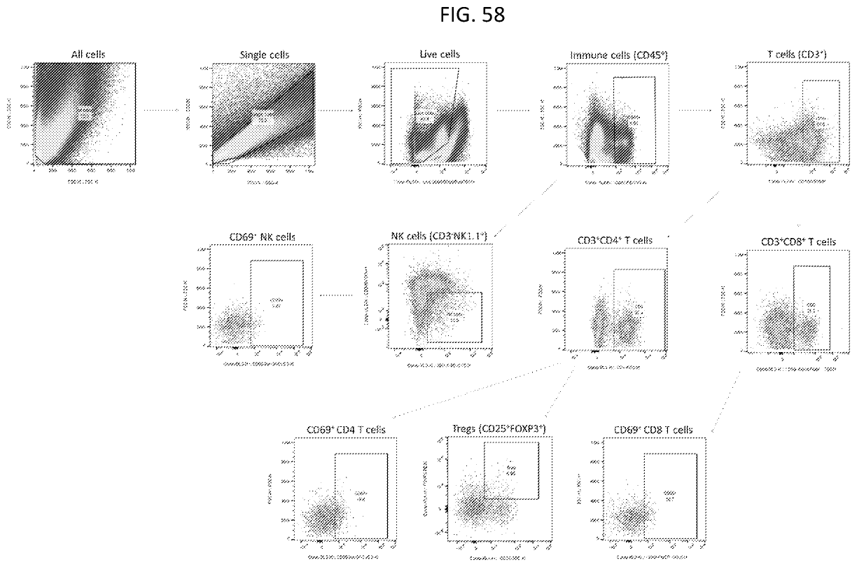

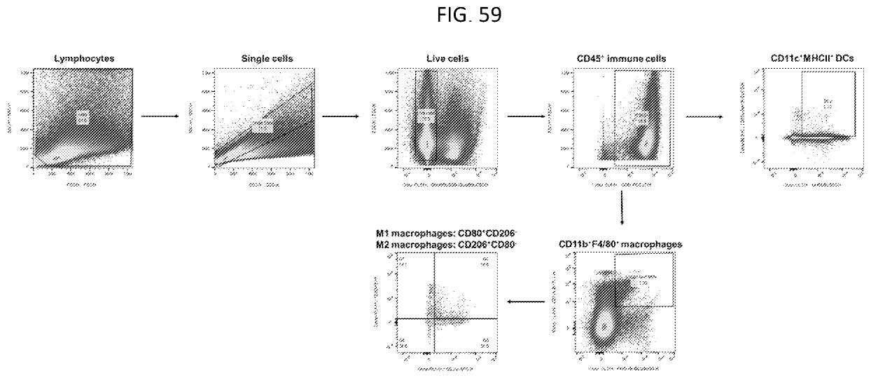

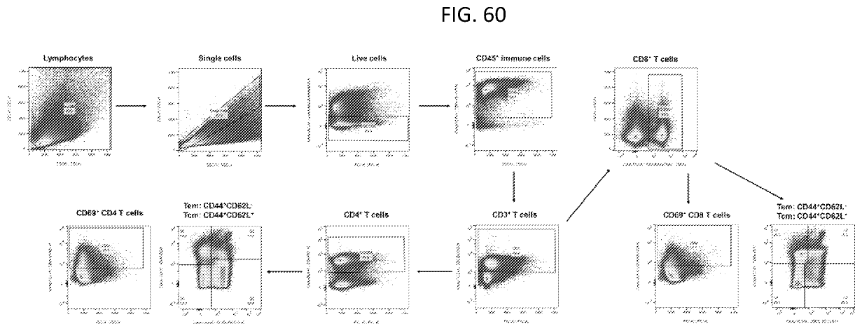

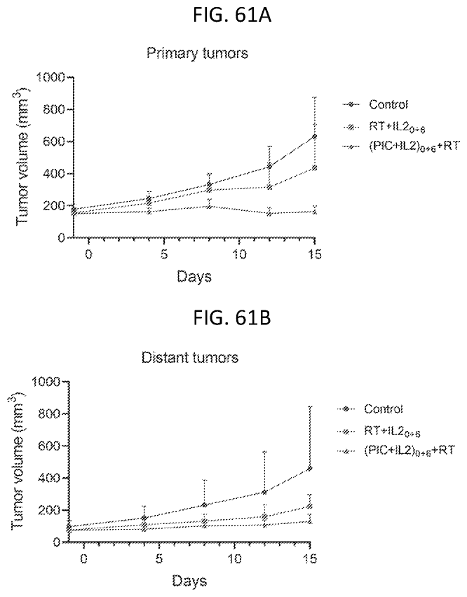

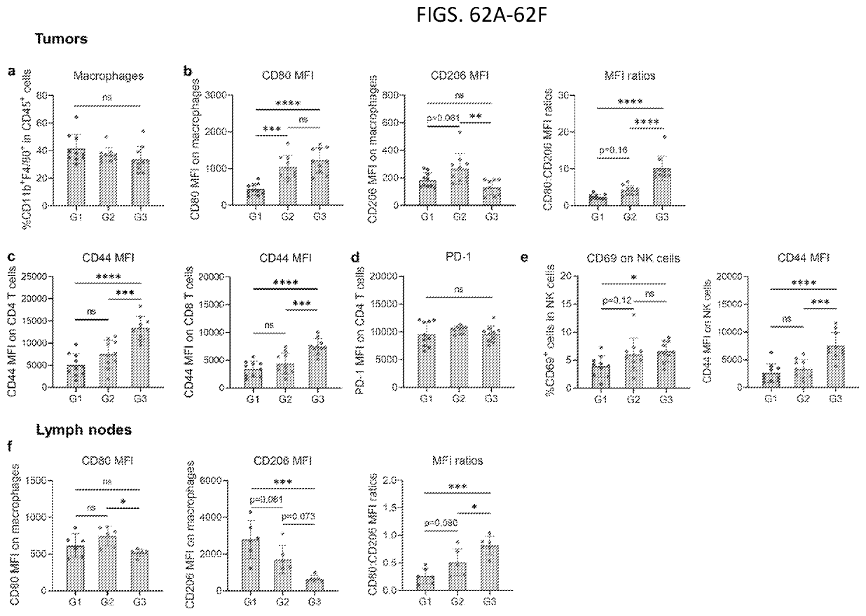

To study the effects of (PIC+IL2)₀₊₆+RT treatment on the tumor immune microenvironment and the specific roles of PIC in this immunomodulation, we analyzed treated tumors and their adjacent tumor-draining lymph nodes (TDLNs) at day 15 following (PIC+IL2)₀₊₆+RT treatment and compared the results with those obtained from RT+IL2₀₊₆ treated or untreated control tumors (FIGS. 46A, 57-60). We found that (PIC+IL2)₀₊₆+RT exhibited superior tumor control for both primary and distant tumors compared to RT+IL2₀₊₆ treatment (FIGS. 61A-61B). H&E images of tumor (FIG. 46B) suggested marked destruction of tumor after (PIC+IL2)₀₊₆+RT treatment. Using flow cytometry to analyze the populations of immune cells infiltrating tumors, we found a similar proportion of CD45⁺ immune cells relative to total live cells in tumors from the (PIC+IL2)₀₊₆+RT and RT+IL2₀₊₆ treatment groups, both of which were significantly higher than the untreated control tumors (FIG. 46C). Although neither the (PIC+IL2)₀₊₆+RT nor the RT+IL2₀₊₆ treatment changed the abundance of CD11b⁺F4/80⁺ macrophages in tumor, PIC injection polarized these macrophages toward CD80⁺CD206⁻ M1 phenotype, leading to higher M1:M2 macrophage ratios in (PIC+IL2)₀₊₆+RT treated tumors compared to RT+IL2₀₊₆-treated or untreated control tumors (FIGS. 46d-46e and 62A-62B). This macrophage polarization effect of PIC was also observed in the primary TDLNs, which may be due to the accumulation of PIC in the TDLNs after intratumoral injection, as we observed previously (FIG. 46K). We evaluated CD11c⁺MHCII⁺ dendritic cells (DCs) and found that (PIC+IL2)₀₊₆+RT reduced the DC population in tumors but upregulated the expression of activation marker, CD80, on these DCs compared to RT+IL2₀₊₆ treatment (FIG. 46F). This downregulation of DCs population in tumor tissues may be associated with the migration of activated antigen-presenting cells (APCs) to the adjacent lymph nodes to prime T cells, as we observed an increased abundance of DCs and macrophages and an enhanced expression of CD69 on T cells in the TDLNs (FIGS. 46K-46M).

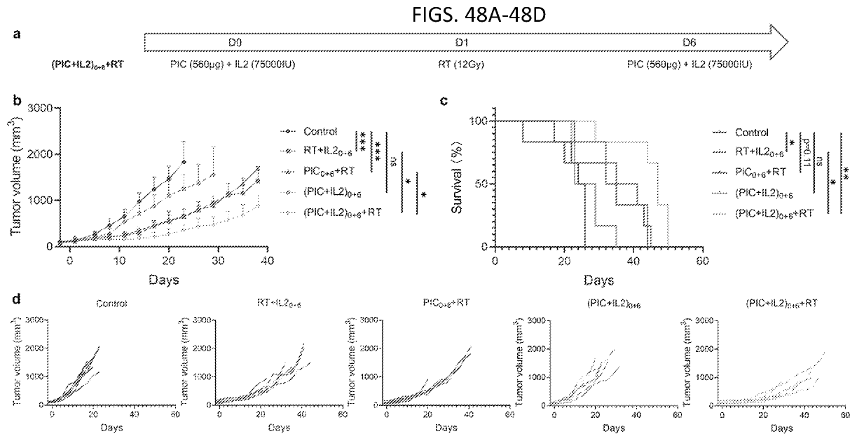

We analyzed tumor infiltrating lymphocytes (TILs) in these B78 melanomas by flow cytometry and immunohistochemistry. We observed enhanced tumor infiltration by CD4⁺ T cells and CD8⁺ T cells after (PIC+IL2)₀₊₆+RT treatment, when compared to untreated control tumors (FIG. 46G). RT+IL2₀₊₆ treatment improved the CD69 expression on both CD4⁺ T cells and CD8⁺ T cells, and this was further increased for CD8⁺ T cells by the addition of PIC (FIG. 48G). The effector marker of T cells, CD44, also significantly increased when PIC was added to the RT+IL2₀₊₆ treatment (FIG. 62C). The PD-1 expression on CD8⁺ T cells was significantly upregulated by RT+IL2₀₊₆, and this effect was reduced with (PIC+IL2)₀₊₆+RT treatment and both of these regimens showed negligible effects on PD-1 expression in CD4⁺ T cells (FIGS. 46G and 62D). PIC injection also reduced the tumor infiltration by Tregs (CD25⁺FOXP3⁺) when added to RT+IL2₀₊₆ treatment (FIG. 46G). Moreover, we found that both the (PIC+IL2)₀₊₆+RT and RT+IL2₀₊₆ treatments significantly increased tumor infiltration by NK cells (NK1.1⁺CD3⁻), and these NK cells exhibited higher expression for CD69 and CD44 in (PIC+IL2)₀₊₆+RT treatment tumors (FIGS. 46H and 62E).

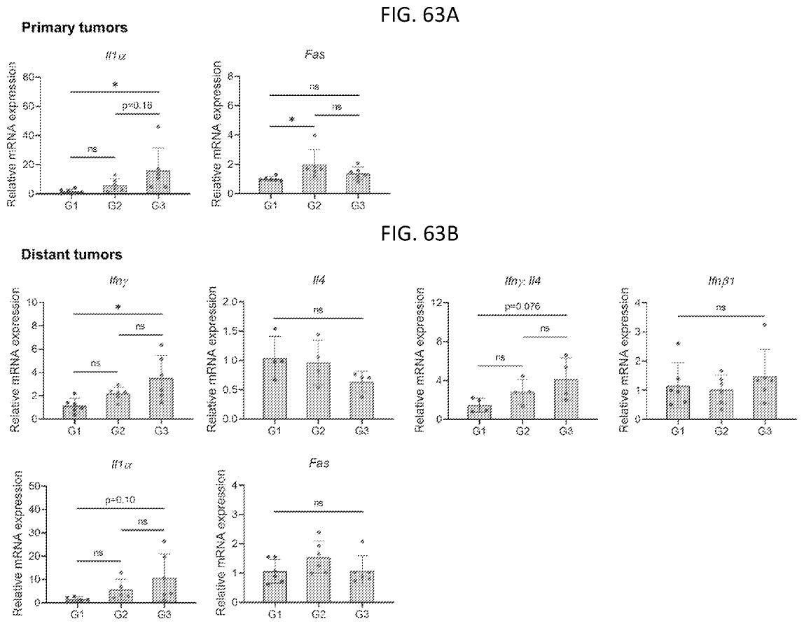

We used RT-qPCR to quantify the expression of cytokines in tumor samples following these treatments. We found that RT+IL2₀₊₆ increased the levels of both Ifnγ and Il4. Addition of PIC injection to this regimen did not change the upregulation of Ifnγ but decreased the Il4 expression to a level that was similar to that observed in untreated control tumors, resulting in higher ratios of Ifnγ. Il4 expression in the (PIC+IL2)₀₊₆+RT group compared to both the control and RT+IL2₀₊₆ groups (FIG. 46I). Further analysis demonstrated that PIC injection did not change the expression of Ifnβ1 or Fas but enhanced the expression of the pro-inflammatory cytokine Il1α in the RT+IL2₀₊₆ treated tumors (FIGS. 46J and 63A).

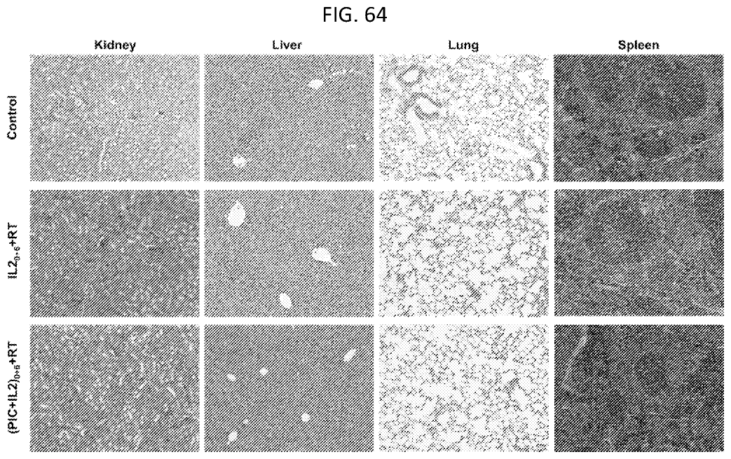

We evaluated memory T cells in the TDLNs from these mice using flow cytometry. As shown in FIGS. 46N, RT+IL2₀₊₆ treatment significantly increased the levels of both effector memory T cells (Tem, CD44⁺CD62L⁻) and central memory T cells (Tcm, CD44⁺CD62L⁺) among CD4⁺ T cells and CD8⁺ T cells, while PIC injection further enhanced the levels of Tcm, which have been reported to exhibited superior persistence and anti-tumor immunity compared to Tem, but showed negligible effects on the generation of Tem. Importantly, histological analysis of vital organs from mice receiving these treatment regimens did not reveal any apparent toxic effect of these treatments on normal tissues including liver, kidney, spleen, and lung (FIG. 64).

To study the systemic anti-tumor immunity generated by the (PIC+IL2)₀₊₆+RT treatment, we collected blood from B78 melanoma two-tumor bearing mice at day 15 after RT and analyzed the circulating T cells. As shown in FIGS. 47A-47B, we found that (PIC+IL2)₀₊₆+RT treatment significantly increased the abundance of CD8⁺ T cells (CD8⁺CD3⁺) and exhibited a trend towards increasing CD4⁺ T cell (CD4⁺CD3⁺) abundance in the blood. (PIC+IL2)₀₊₆+RT treatment increased the expression of CD44 and NKG2D on the CD8⁺ T cells, while no changes of these markers were observed for CD4⁺ T cells (FIGS. 47A-47B). NKG2D has been reported as one of the markers for circulating tumor specific T cells, while CD44 is a marker for effector T cells. These results indicated that (PIC+IL2)₀₊₆+RT treatment can augment the systemic anti-tumor CD8⁺ T cell response, consistent with our observation of effective suppression of distant tumor sites.

Since the (PIC+IL2)₀₊₆+RT treatment was demonstrated to generate a systemic T cell response and exhibited considerable effects in inhibiting the growth of distant tumors (FIGS. 45A-45J and 47A-47H), we analyzed the distant B78 melanomas to determine how the local treatment of (PIC+IL2)₀₊₆+RT modulated the immune cells infiltration in distant tumors that did not receive direct treatment. As shown in FIG. 47C, RT+IL2₀₊₆ treated mice exhibited similar CD45⁺ immune cell infiltration in distant tumors when compared to those tumors in untreated control mice whereas (PIC+IL2)₀₊₆+RT treatment significantly enhanced this tumor immune cell infiltration. A similar trend was observed for the tumor infiltrating T cells, as both flow cytometry and immunohistochemistry suggested that the local RT+IL2₀₊₆ treatment did not change the infiltration of CD4⁺ T cells and CD8⁺ T cells in distant tumors, but the infiltration of these cells significantly increased when PIC was included with the RT+IL2₀₊₆ treatment (FIGS. 47D-47E).

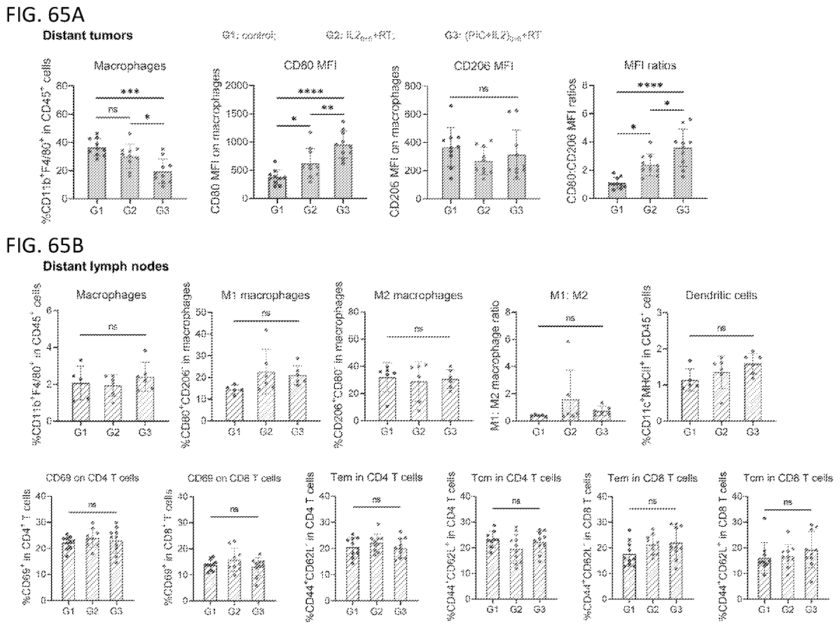

Given that analysis of the blood indicated a functional effect on circulating CD8⁺ T cells after (PIC+IL2)₀₊₆+RT treatment, we analyzed functional markers on CD8⁺ T cells in the distant tumors. As shown in FIG. 47F, (PIC+IL2)₀₊₆+RT treatment significantly reduced the expression of the exhaustion marker, PD-1, on the CD8⁺ T cells, although the treatment did not affect the expression of CD69 or CD44 on CD8⁺ T cells when compared to RT+IL2₀₊₆ treatment. Intriguingly, we found that (PIC+IL2)₀₊₆+RT treatment downregulated the abundance of CD11b⁺F4/80⁺ macrophages in distant tumors and repolarized these TAMs toward CD80⁺CD206⁻ M1 phenotypes, leading to higher M1:M2 macrophage ratios in distant tumors (FIGS. 47G and 65A). (PIC+IL2)₀₊₆+RT and RT+IL2₀₊₆ treatments did not change the infiltration of the CD11c⁺MHCII⁺ DCs in distant tumors, while RT+IL2₀₊₆ treatment increased CD80 expression on these DCs and PIC further increased this (FIG. 47H).

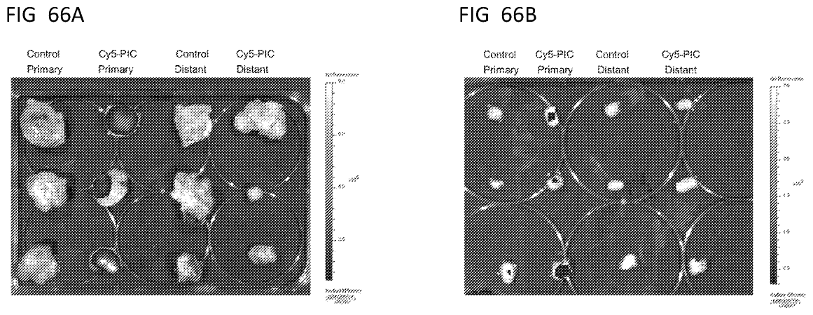

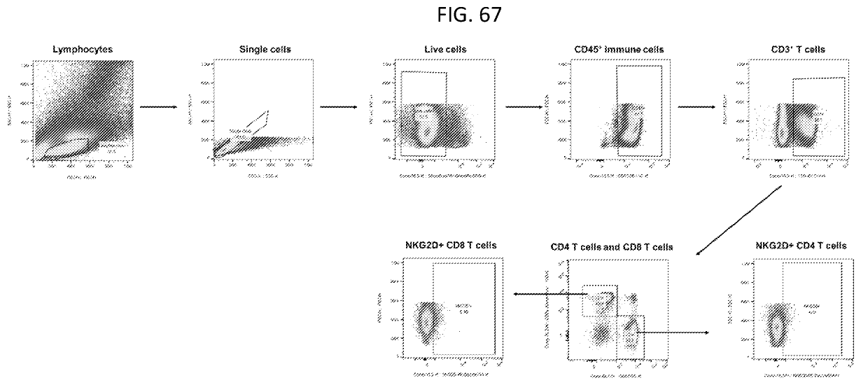

Quantification of gene expression from bulk tumor by RT-qPCR (FIG. 63B) demonstrated increased expression ratios of Ifnγ. Il4 and increased levels of pro-inflammatory cytokine Il1α in distant tumors from (PIC+IL2)₀₊₆+RT treated mice compared to those from untreated control mice, and this treatment showed negligible effects on the expression of Ifnβ1 and Fas at these distant tumor sites. To test whether this immunomodulation of distant tumors resulted from the biodistribution of PIC in distant tumors, we injected the Cy5-labeled PIC to the primary (right) tumor on B78 melanoma two-tumor bearing mice and studied its biodistribution in tumors and TDLNs. At day 15 after the first injection, we observed strong fluorescence signals of Cy5-PIC in the primary tumors and primary TDLNs, but not the distant tumor and distant TDLNs (FIG. 66). Flow cytometry analysis of distant TDLNs also revealed no effects induced by contralateral PIC treatment (FIG. 65B). These results indicated that the immunomodulation of distant tumors induced by (PIC+IL2)₀₊₆+RT treatment may be mainly attributed to the generation of systemic anti-tumor immunity.

(PIC+IL2)₀₊₆+RT treatment in the murine MOC2 head and neck squamous cell carcionoma model.

One of the unique features of an in situ vaccine approach is that identification of tumor neoantigens is not required to achieve therapeutic efficacy and therefore the efficacy of such a regimen is not limited to a given disease type or by the expression of a given tumor antigen.³⁸ We tested the (PIC+IL2)₀₊₆+RT treatment on another immunologically “cold” and aggressive MOC2 head and neck squamous cell carcinoma (FIG. 48A). (PIC+IL2)₀₊₆+RT treated tumors exhibited slower tumor growth resulting in improved mice survival compared to those treated with the doublet combinations of RT+IL2₀₊₆, PIC₀₊₆+RT or (PIC+IL2)₀₊₆ (FIG. 48B-48D).

### DISCUSSION

Cancer vaccines are promising methods for the treatment of solid tumors yet traditional cancer vaccines are limited by the requirement for identification and specific targeting of a known tumor-associated antigen (TAA). An in situ vaccine can bypass this limitation and enable cross presentation of TAAs in situ, without requiring identification of these, in order to activate an adaptive immune response. RT has been demonstrated to stimulate an in situ vaccination effect, however this has not yet been potent enough to stimulate a clinically meaningful improvement in systemic anti-tumor immunity when added to immune checkpoint blockade.^{12, 40} This may reflect inadequate priming of an adaptive T cell immunity and the stimulation of immunosuppressive mechanisms by RT. We introduced a multifunctional nanoparticle (PIC) to address these limitations. While that agent improved the in situ vaccine effect of RT when combined with immune checkpoint blockade, it was not sufficient to stimulate a de novo anti-tumor immune response in the absence of systemic immune checkpoint inhibition. We hypothesized that may reflect a limitation of the PIC, which enabled more effective adaptive immune priming and blunted potential suppressive effects of RT on the tumor microenvironment, but which did not incorporate a stimulus to enhance clonal expansion of the stimulated immune response. To redress this, here we combined PIC with intratumoral IL2 and RT.

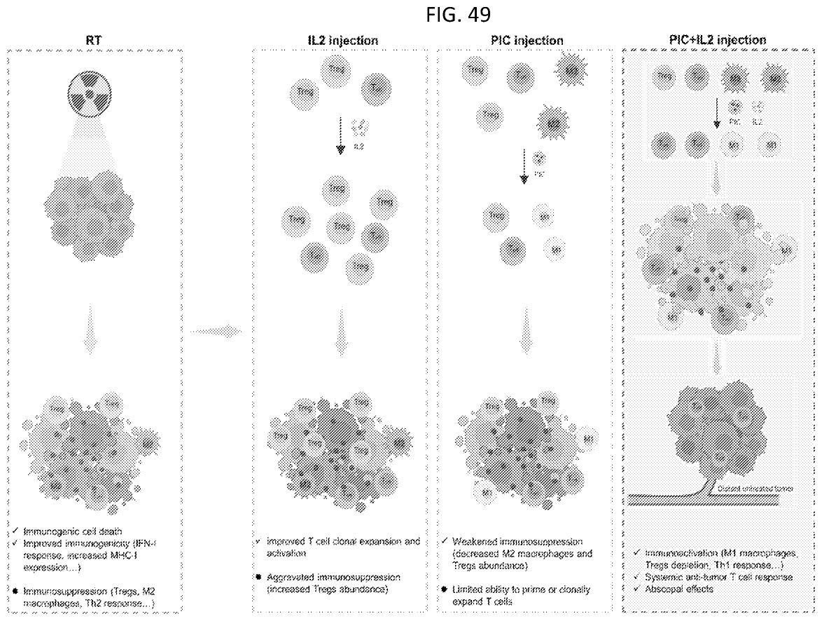

We observed that the triple combination of PIC+RT+IL2 was effective in stimulating anti-tumor immune response in the immunologically “cold” B78 and MOC2 tumor models, even in the absence of systemic immune checkpoint inhibition. In evaluating the mechanisms of cooperative therapeutic interaction between these locally administered therapies, we observed that PIC prevented specific detrimental effects caused by RT+IL2 and strengthened select favorable effects, resulting in a potent systemic anti-tumor immunity that improved tumor eradication and mouse survival (FIG. 49). More specifically, we found that PIC coordinated with IL2 to promote Th1 polarization of the adaptive immune response and M1 polarization of macrophages (FIGS. 43A-43K). These effects of PIC relieved local immunosuppression and improved the T cells activation following RT+IL2, facilitating the generation of potent adaptive antitumor immunity at tumors not directly received any treatment. This may be related to not only the immunomodulation of the radiated TME by PIC, but also the enhanced antigen presentation by PIC, as we demonstrated previously.

By optimizing the PIC+RT+IL2 treatment regimen, we found that injection of PIC+IL2 at days 0 and 6 relative to RT exhibited a similar induction of systemic antitumor immune response when compared with a more frequent injection schedule (FIGS. 45A-45J). Due to the translational challenges posed by a need for repeated intratumoral injections in clinical, we then focused on the (PIC+IL2)₀₊₆+RT treatment regimen and tested it in confirmed its efficacy in the MOC2 head and neck tumor model. Specifically, (PIC+IL2)₀₊₆+RT suppressed the growth of MOC2 tumors and prevented development of lung metastasis. We further demonstrated that this therapeutic efficacy could be augmented by additional combination with immune checkpoint inhibition (FIGS. 48A-48H).

Although PIC did not exhibit any direct effects on the Tregs in vitro, in vivo studies demonstrated that PIC significantly lowered the abundance of Tregs in tumor tissues and downregulated PD-1 expression on CD8⁺ T cells (FIGS. 46A-46N). Analyses of distant tumors that were not directly treated with RT or intratumoral injection revealed that PIC facilitated immunoregulation of these distant TMEs, leading to increased levels of tumor-infiltrating lymphocytes, higher M1:M2 macrophages ratios, and stimulation of Th1 polarized adaptive immune response (FIGS. 47A-47H). This immunomodulation of distant untreated tumors was not directly stimulated by PIC, which exhibited biodistribution limited to the injected tumor and its TDLN, but may be related to the circulation of tumor-specific T cells after (PIC+IL2)₀₊₆+RT treatment (FIGS. 66 and 47A-47H). These indicated that PIC is effective in both direct local tumor immunomodulation and indirect systemic modulation of tumors secondary to the generation of systemic T cell immunity.

### REFERENCES

[1] C. X. Li, Y. Zhang, X. Dong, L. Zhang, M. D. Liu, B. Li, M. K. Zhang, J. Feng, X. Z. Zhang, Artificially Reprogrammed Macrophages as Tumor-Tropic Immunosuppression-Resistant Biologics to Realize Therapeutics Production and Immune Activation, Adv Mater 31(15) (2019) e1807211.
[2] M. Haraguchi, S. Yamashiro, A. Yamamoto, K. Furukawa, K. Takamiya, K. O. Lloyd, H. Shiku, K. Furukawa, Isolation of GD3 synthase gene by expression cloning of GM3 a-2,8-sialyltransferase cDNA using anti-GD2 monoclonal antibody, Natl Acad Sci USA, 91 (1994), 10455-10459.
[3] F. Jallow, K. A. O'Leary, D. E. Rugowski, J. F. Guerrero, S. M. Ponik, L. A. Schuler, Dynamic interactions between the extracellular matrix and estrogen activity in progression of ER+ breast cancer, Oncogene 38(43) (2019) 6913-6925.
[4] Morris, Z. S. et al. In situ tumor vaccination by combining local radiation and tumor-specific antibody or immunocytokine treatments. Cancer Res. 76, 3929-3941, (2016).
[5] N. A. Franken, H. M. Rodermond, J. Stap, J. Haveman, C. van Bree, Clonogenic assay of cells in vitro, Nat Protoc 1(5) (2006) 2315-9.
[6] K. A. Pilones, M. Charpentier, E. Garcia-Martinez, C. Daviaud, J. Kraynak, J. Aryankalayil, S. C. Formenti, S. Demaria, Radiotherapy Cooperates with IL15 to Induce Antitumor Immune Responses, Cancer Immunol Res 8(8) (2020) 1054-1063.

### EQUIVALENTS

While certain embodiments have been illustrated and described, a person with ordinary skill in the art, after reading the foregoing specification, can effect changes, substitutions of equivalents and other types of alterations to the nanoparticles of the present technology or derivatives, prodrugs, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Likewise, the use of the terms “comprising,” “including,” “containing,” etc. shall be understood to disclose embodiments using the terms “consisting essentially of” and “consisting of” and vice versa. The phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

1. A nanoparticle comprising polylysine electrostatically bound to iron oxide nanoparticles and CpG oligodeoxynucleotide, wherein the nanoparticle has a hydrodynamic diameter from about 50 to about 200 nm and a zeta potential from +10 mV to +60 mV.

2. The nanoparticle of claim 1, wherein the iron oxide is Fe₃O₄, FeO, iron (II,III) oxide, or Fe₂O₃.

3. The nanoparticle of claim 1, wherein the iron oxide comprises from about 30 wt % to about 80 wt % of the nanoparticle.

4. The nanoparticle of claim 3, wherein the iron oxide comprises from about 45 wt % to about 70 wt % of the nanoparticle.

5. The nanoparticle of claim 1, wherein the polylysine comprises about 5 wt % to about 50 wt %.

6. The nanoparticle of claim 5, wherein the polylysine comprises about 10 wt % to about 40 wt %.

7. The nanoparticle of claim 1, wherein the polylysine has a molecular weight of 30 kDa to 70 kDa.

8. The nanoparticle of claim 1, wherein CpG oligodeoxynucleotide comprises about 5 wt % to about 30 wt %.

9.-12. (canceled)

13. The nanoparticle of claim 1, further comprising a label.

14. The nanoparticle of claim 13, wherein the label is selected from the group consisting of dyes, radioisotope chelators for PET imaging, and chelators for MRI imaging.

15. The nanoparticle of claim 13, wherein the label is conjugated to at least a portion of PLL.

16.-19. (canceled)

20. A method of treatment comprising administering to a subject suffering from a cancer an effective amount of a nanoparticle of claim 1 and an effective amount of radiation therapy.

21. The method of claim 20, wherein the cancer is an immunologically cold cancer.

22. The method of claim 20, wherein the cancer is an immunologically hot cancer.

23. The method of claim 20, wherein the treatment increases the expression of pro-inflammatory cytokines.

24. The method of claim 20, wherein the treatment increases T cell activation.

25. The method of claim 1 further comprising administering an effective amount of a checkpoint inhibitor and/or immune adjuvant to the subject.

26.-28. (canceled)

29. The method of claim 25, wherein the checkpoint inhibitor is a CTLA4 inhibitor, PD1 inhibitor, or PDL1 inhibitor.

30. The method of claim 25, wherein the immune adjuvant is a cytokine adjuvant.

31. The method of claim 30, wherein the cytokine adjuvant is interleukin-2 (IL-2).

32.-35. (canceled)
